SEQUENCE LISTING (70 sequences)

SEQ ID NO: 1 - DNA, 5138 bp, Hepatitis E virus JRA1
ggcagaccacgtatgtggtcgatgccatggaggcccaccagttcattaaggctcctggcattactactgccatagagcaggctgctctggctgcggccaactccgccttggcgaatgctgtggtggttcggccgtttttgtctcgcgtacaaaccgagatccttattaatttgatgcaaccccggcagttggttttccgccctgaggtgctttggaaccatcctattcagagggttatacacaatgaattagaacagtactgccgggcccgggccggtcgttgcctggagattggggctcatccaagatctattaatgataaccccaatgttttgcaccggtgttttctcaggccggtcggtagggacgtccagcgctggtattctgcccccacccgcggccctgcagccaactgccgccgctctgcattgcgtggcctcccccctgtcgaccgcacttactgttttgatggattttccagttgtgctttcgccgcagagaccggcgtggccctttactctctacatgacctttggccagctgatgttgcggaggctatggcccgccatggaatgacacgtctttatgctgcactccacctccctcccgaggtgttgttaccacccggtacttaccacacaacctcgtatcttctgatccatgacggcaaccgtgctgttgtgacttatgaaggtgatactagtgcaggttacaaccatgacgtttccatacttcgtgcatggattcgcacgactaaaatagttggtgaccatccgttggttatagagcgtgtgcgggctattggttgtcattttgtgctgctgctcaccgcggcccctgagccatcgcccatgccttacgtcccttacccccgttccacggaggtgtatgtccgatctatatttggccctggtggctccccatccttgtttccgtcagcttgctctacaaaatctaccttccatgctgttccagttcacatatgggaccggcttatgctttttggtgccaccttggacgaccaggcgttttgctgctcacggctcatgacatacctccgtggcattagctacaaagtcactgtcggtgcgcttgttgctaatgaggggtggaatgcctctgaagatgccctcaccgcagtaatcactgcagcctacttgaccatctgtcaccagcgctacctccgtacccaggcgatatctaagggtatgcgccggttggaggttgagcacgcccagaagtttattacaagactttacagctggttatttgagaagtctggtcgtgactacatccccggccgccaacttcagttctatgcccagtgccggcggtggctatctgcgggctttcacctagaccccagagtgctcgtctttgatgagtcagtgccctgccgctgtaggactttcttaaagaaggttgcgggtaaattctgttgcttcatgcggtggttagggcaggagtgtacttgtttcttagaaccagctgagggcttagttggcgatcaaggccatgataatgaggcttatgagggttctgaggttgaccaggccgagcctgtacaccttgatgtgtcggggacctatgctgtccacgggcaccagcttgaggccctctacagggcactcaacattccacacgatattgctgctcgagctgcccgattaacggctactgttgagctcgctgcaggccccgaccgtctggagtgccgcaccgtgctcgggaacaagactttccggacgacggtgactgatggcgcccatctagaagcgaacggccctgagcaatatgttctgtcgttcgatgcctcccgccagtctatgggggccgggtcccatagcctcacttacgagcttacacccgccggcttgcaggttaagatctcatctaatggcctagactgcactgccgtattcccccctggtggcgcccctagcgcgccgccgggggaggtggcggctttttgcagcgccctctacaggtataacagattcacccagcggcattcgctgaccggcggtttgtggctacatcccgaggggttgctaggcgttttcccccctttttcccctgggcacatttgggagtctgccaaccccttctgcggtgagggtaccttgtatactcgcacctggtctacatctggtttttctagtgacttttcccctcctgaggcggccgcccctgcaccggctgccgccccagggtcgtcctcccccactccaccagtcagtgatatttgggtgttaccaccgctttcagaggagccccaggtgggtgcgccgcctgcacctcccacccccgagcctgctcggctgccctgccccactaaacctaacacccccgtgcgtaagccaacggcaccgccgccttctcgcacccgccgccttctttacacctatcctgatggtgctaaggtgtatgcggggtcactgtttgagtcagattgtgattggctggtgaatgcctctaacccgggccatcgtcctgggggtggcctctgtcacgccttttaccaacgttttcccgaggcgttctatccaactgaatttattatgcgtgagggtcttgcagcatacaccttgaccccgcgccctattatccatgcagtggcccccgactatagggttgagcagaatccaaagaggcttgaggcagcgtaccgagaaacttgctcccgccgcggcaccgctgcctatccacttcttggctcaggtatataccaggtccctgctggtcttagttttgatgcctgggagcgtaaccatcgccctggcgatgagctttacctgactgaacccgctgcagcctggtttgaagctaataagccaacgcagccagcacttacgataacggaagatacagctcgcacggccaacctagcattagagatcgatgctgccacagacgttggccgtgcttgtgccggctgcactattagccccgggattgtgcattatcagttcactgccggggtcccaggctcaggcaaatcaaggtccatacagcagggggatgtcgatgttgtggttgtccccacccgggaacttcgcaatagctggcgccgtcggggctttgcggcctttacaccccacacggcggcccgtgttactataggccgccgtgttgttattgacgaggccccatcccttccaccgcacttgttgctgttacacatgcagcgggcctcctcagtccatctcctcggtgacccaaatcagatccctgccatcgacttcgagcatgccggcctggtccccgcaattcgccctgagcttgcaccaacgagctggtggcatgttacgcatcgctgtccggccgatgtatgtgagctcatacgcggagcctaccctaaaatccagaccacgagccgtgtgctacggtctctgttctggaatgagccggctattggccagaagttggtcttcacgcaggccgctaaagctgccaaccctggtgcaattacggtccatgaagcccagggtgctaccttcacagagaccacaattatagccacggctgatgccaggggtcttattcagtcatcccgggcccatgctatagttgcacttacccgccacacagagaagtgcgttattttggatgcccctggtttgttgcgcgaggtcggcatatcagatgtaattgttaacaactttttccttgctggcggagaggtgggccatcaccgcccctctgtgatacctcgtggcaatcctgaccggaaccttgacaccctacaggccttcccgccatcctgccaaattagtgcttaccatcagttggctgaggagttaggccatcgcccggctcctgttgctgccgttttgcccccctgccccgagctcgagcagggcttattgtatatgccacaggagcttacagtgtccgatagtgtgttggtttttgagctcactgacatagtccattgccgcatggctgccccaagtcagcggaaggccgtcctctcaacacttgtggggaggtatggccgcaagacgaaattgtatgaggcagcccattcagatgttcgagagtctctagctaggttcattcccactatcggacctgtccaggccaccacgtgtgagttatatgaactggttgaggccatggtggaaaagggccaggacggctctgccgtcctggagcttgatctatgtaatcgtgatgtttcgcgcattacattctttcagaaagactgtaataagtttacaactggtgagaccattgcccatggtaaggttggccagggcatatcggcctggagtaagaccttttgcgccctgtttggtccgtggtttcgtgccattgaaaaagaaatactagccctgctcccgcctaatatcttctacggcgacgcctacgaggagtcggtgtttgccgcggccgtgtccggggcggggtcttgcatggtatttgaaaatgacttttcggaatttgatagtactcagaacaatttctcccttggccttgagtgtgtggttatggaagagtgtggtatgccccaatggctgatcagattgtatcacctggtccggtcagcctggattctgcaggcgccaaaggagtctcttaaaggtttctggaagaagcattctggtgagcctggtacccttctctggaacaccatctggaacatggcgatcatagcccattgctatgagtttcgcgatttccgggtcgccgcttttaagggtgatgactcggtagtcctctgtagtgattaccgacagagtcgtaacgcggcagccttaatcgcaggttgtgggctcaagttgaaggttgactatcgccctattgggctgtacgctggtgtggtggtggcccctggcttggggacactgcctgatgtagtgcgatttgctggccggctgtccgaaaagaattggggccctggcccagagcgtgctgagcagctgcgtcttgctgtctgtgacttccttcgagggttaacgaatgttgcgcaggtttgtgttgatgttgtgtcccgtgtttatggagtcagccccgggctggtacataaccttattggcatgctgcagaccattgctgatggcaaggcccactttacagagtcaattaaacctgtgcttgaccttacaaattctattatacagcgggtggaatga

SEQ ID NO: 2 - DNA, 15 bp, Artificial
  Synthetic DNA
tgccatggaggccca

SEQ ID NO: 3 - DNA, 15 bp, Artificial
  Synthetic DNA
tkgcgaatgctgtgg

SEQ ID NO: 4 - DNA, 15 bp, Artificial
  Synthetic DNA
rcagtargtgcggtc

SEQ ID NO: 5 - DNA, 15 bp, Artificial
  Synthetic DNA
cctcsgcracatcag

SEQ ID NO: 6 - DNA, 15 bp, Artificial
  Synthetic DNA
gtcggtagggacgtc

SEQ ID NO: 7 - DNA, 2442 bp, Hepatitis E virus
tcgatgccatggaggcccaccagttcattaaggctcctggcattactactgccatagagcaggctgctctggctgcggccaactccgccttggcgaatgctgtggtggttcggccgtttttgtctcgcgtacaaaccgagatccttattaatttgatgcaaccccggcagttggttttccgccctgaggtgctttggaaccatcctattcagagggttatacacaatgaattagaacagtactgccgggcccgggccggtcgttgcctggagattggggctcatccaagatctattaatgataaccccaatgttttgcaccggtgttttctcaggccggtcggtagggacgtccagcgctggtattctgcccccacccgcggccctgcagccaactgccgccgctctgcattgcgtggcctcccccctgtcgaccgcacttactgttttgatggattttccagttgtgctttcgccgcagagaccggcgtggccctttactctctacatgacctttggccagctgatgttgcggaggctatggcccgccatggaatgacacgtctttatgctgcactccacctccctcccgaggtgttgttaccacccggtacttaccacacaacctcgtatcttctgatccatgacggcaaccgtgctgttgtgacttatgaaggtgatactagtgcaggttacaaccatgacgtttccatacttcgtgcatggattcgcacgactaaaatagttggtgaccatccgttggttatagagcgtgtgcgggctattggttgtcattttgtgctgctgctcaccgcggcccctgagccatcgcccatgccttacgtcccttacccccgttccacggaggtgtatgtccgatctatatttggccctggtggctccccatccttgtttccgtcagcttgctctacaaaatctaccttccatgctgttccagttcacatatgggaccggcttatgctttttggtgccaccttggacgaccaggcgttttgctgctcacggctcatgacatacctccgtggcattagctacaaagtcactgtcggtgcgcttgttgctaatgaggggtggaatgcctctgaagatgccctcaccgcagtaatcactgcagcctacttgaccatctgtcaccagcgctacctccgtacccaggcgatatctaagggtatgcgccggttggaggttgagcacgcccagaagtttattacaagactttacagctggttatttgagaagtctggtcgtgactacatccccggccgccaacttcagttctatgcccagtgccggcggtggctatctgcgggctttcacctagaccccagagtgctcgtctttgatgagtcagtgccctgccgctgtaggactttcttaaagaaggttgcgggtaaattctgttgcttcatgcggtggttagggcaggagtgtacttgtttcttagaaccagctgagggcttagttggcgatcaaggccatgataatgaggcttatgagggttctgaggttgaccaggccgagcctgtacaccttgatgtgtcggggacctatgctgtccacgggcaccagcttgaggccctctacagggcactcaacattccacacgatattgctgctcgagctgcccgattaacggctactgttgagctcgctgcaggccccgaccgtctggagtgccgcaccgtgctcgggaacaagactttccggacgacggtgactgatggcgcccatctagaagcgaacggccctgagcaatatgttctgtcgttcgatgcctcccgccagtctatgggggccgggtcccatagcctcacttacgagcttacacccgccggcttgcaggttaagatctcatctaatggcctagactgcactgccgtattcccccctggtggcgcccctagcgcgccgccgggggaggtggcggctttttgcagcgccctctacaggtataacagattcacccagcggcattcgctgaccggcggtttgtggctacatcccgaggggttgctaggcgttttcccccctttttcccctgggcacatttgggagtctgccaaccccttctgcggtgagggtaccttgtatactcgcacctggtctacatctggtttttctagtgacttttcccctcctgaggcggccgcccctgcaccggctgccgccccagggtcgtcctcccccactccaccagtcagtgatatttgggtgttaccaccgctttcagaggagccccaggtgggtgcgccgcctgcacctcccacccccgagcctgctcggctgccctgccccactaaacctaacacccccgtgcgtaagccaacggcaccgccgccttctcgcacccgccgccttctttacacctatcctgatggtgctaaggtgtatgcggggtcactgtttgagtcagattgtgatt

SEQ ID NO: 8 - protein, 1703 aa, Hepatitis E virus
MEAHQFIKAPGITTAIEQAALAAANSALANAVVVRPFLSRVQTEILINLMQPRQLVFRPEVLWNHPIQRVIHNELEQYCRARAGRCLEIGAHPRSINDNPNVLHRCFLRPVGRDVQRWYSAPTRGPAANCRRSALRGLPPVDRTYCFDGFSSCAFAAETGVALYSLHDLWPADVAEAMARHGMTRLYAALHLPPEVLLPPGTYHTTSYLLIHDGNRAVVTYEGDTSAGYNHDVSILRAWIRTTKIVGDHPLVIERVRAIGCHFVLLLTAAPEPSPMPYVPYPRSTEVYVRSIFGPGGSPSLFPSACSTKSTFHAVPVHIWDRLMLFGATLDDQAFCCSRLMTYLRGISYKVTVGALVANEGWNASEDALTAVITAAYLTICHQRYLRTQAISKGMRRLEVEHAQKFITRLYSWLFEKSGRDYIPGRQLQFYAQCRRWLSAGFHLDPRVLVFDESVPCRCRTFLKKVAGKFCCFMRWLGQECTCFLEPAEGLVGDQGHDNEAYEGSEVDQAEPVHLDVSGTYAVHGHQLEALYRALNIPHDIAARAARLTATVELAAGPDRLECRTVLGNKTFRTTVTDGAHLEANGPEQYVLSFDASRQSMGAGSHSLTYELTPAGLQVKISSNGLDCTAVFPPGGAPSAPPGEVAAFCSALYRYNRFTQRHSLTGGLWLHPEGLLGVFPPFSPGHIWESANPFCGEGTLYTRTWSTSGFSSDFSPPEAAAPAPAAAPGSSSPTPPVSDIWVLPPLSEEPQVGAPPAPPTPEPARLPCPTKPNTPVRKPTAPPPSRTRRLLYTYPDGAKVYAGSLFESDCDWLVNASNPGHRPGGGLCHAFYQRFPEAFYPTEFIMREGLAAYTLTPRPIIHAVAPDYRVEQNPKRLEAAYRETCSRRGTAAYPLLGSGIYQVPAGLSFDAWERNHRPGDELYLTEPAAAWFEANKPTQPALTITEDTARTANLALEIDAATDVGRACAGCTISPGIVHYQFTAGVPGSGKSRSIQQGDVDVVVVPTRELRNSWRRRGFAAFTPHTAARVTIGRRVVIDEAPSLPPHLLLLHMQRASSVHLLGDPNQIPAIDFEHAGLVPAIRPELAPTSWWHVTHRCPADVCELIRGAYPKIQTTSRVLRSLFWNEPAIGQKLVFTQAAKAANPGAITVHEAQGATFTETTIIATADARGLIQSSRAHAIVALTRHTEKCVILDAPGLLREVGISDVIVNNFFLAGGEVGHHRPSVIPRGNPDRNLDTLQAFPPSCQISAYHQLAEELGHRPAPVAAVLPPCPELEQGLLYMPQELTVSDSVLVFELTDIVHCRMAAPSQRKAVLSTLVGRYGRKTKLYEAAHSDVRESLARFIPTIGPVQATTCELYELVEAMVEKGQDGSAVLELDLCNRDVSRITFFQKDCNKFTTGETIAHGKVGQGISAWSKTFCALFGPWFRAIEKEILALLPPNIFYGDAYEESVFAAAVSGAGSCMVFENDFSEFDSTQNNFSLGLECVVMEECGMPQWLIRLYHLVRSAWILQAPKESLKGFWKKHSGEPGTLLWNTIWNMAIIAHCYEFRDFRVAAFKGDDSVVLCSDYRQSRNAAALIAGCGLKLKVDYRPIGLYAGVVVAPGLGTLPDVVRFAGRLSEKNWGPGPERAEQLRLAVCDFLRGLTNVAQVCVDVVSRVYGVSPGLVHNLIGMLQTIADGKAHFTESIKPVLDLTNSIIQRVE

SEQ ID NO: 9 - DNA, 19 bp, Artificial
  Synthetic DNA
ttctttctggccccagact

SEQ ID NO: 10 - DNA, 19 bp, Artificial
  Synthetic DNA
ttctttctgaccccagact

SEQ ID NO: 11 - DNA, 7234 bp, Hepatitis E virus JSN-FH
gtcgacgccatggaggcccatcagttcataaaggctcctggcgtcacaactgctattgagcaggcagctctagcagcggccaactccgccttggcgaatgctgtggtggttcggccttttctgtcccggctacagacagagatacttattaacttgatgcagccccggcagcttgttttccggcctgaggtcctgtggaatcaccccattcaacgcgtgatccacaatgagcttgaacagtactgtcgggctcgggccggccgctgtcttgaagtgggcgctcacccacgttcgatcaacgacaaccccaacgtcttgcatcgctgttttttaaaaccagttggccgcgatgtccagcggtggtacaccgctcctacccgcggccctgcagcgaactgccgccggtcagcccttcgtggcctcccgcctgttgaccggacatattgctttgacggtttttccggttgcacatttgccgccgagacggggattgccctctactcactgcatgacctttggcctgccgatgttgctgaggcgatggcccgccacggtatgacccggctgtatgcggctctccacctccctcctgaggtgctgctcccccctggtacctatcataccacctcgtacctactcatccatgatggggaccgtgcagtgattacatatgagggggattctagtgctgggtacaaccatgatgtgtccatcttgcgcgcctggatccgcactactaaggttactggtgaccacccgctggtgattgagcgagttcgggcggtgggttgtcattttgtgctccttctaacagctgcacctgaaccatcgccaatgccctatgttccataycctcgttccactgaggtttacgttcggtccatttttggccctggtggttcaccatccctgtttccaactgcctgctctactaagtcaacatttcatgctgtccctgtgcatatatgggacagacttatgttattcggtgcgaccctcgacgatcaggccttttgttgctcgaggctaatgacatacctccgtggcattagctacaaagtcacggtcggcgctcttgttgccaacgagggctggaatgcctccgaggatgcattgaccgctgtgattactgcggcctatcttaccatttgccatcagaggtaccttcgcacgcaggccatctcgaaagggatgaaaagactggagcttgagcacgcacagaagttcataacacgtctttacagctggttgtttgaaaaatccggccgtgattacattcccggccgtcagctgcagttttatgcccagtgccgccgatggctttccgctggtttccatcttgatcctcgtgtgcttgtctttgatgaagcggccccttgtcgctgccgtagtttccttcgtaaagctgctcatagattttgttgttttatgcggtggctgggccaggattgtacctgtttcctccagcccgttgaggggagggttggcgagcagggttacgataatgaggcatttgaggggtcggatgttgaccccgctgaggaggccactgtgaacatctctggatcatatattgtcaccggtagccagctacagcctctctaccaggcgcttggcatcccttctgatcttgccgcccgagcgggtcgacttactgccactgttgaagtcctagatgctgatggccgcctcacttgtaagaccactattggcaataagacctttacaacagtttttactgatggtgctcagctagaggctaacgggccggagcagtatgtgctgtcgtttgactcggccaagcaaactatggccgccggcccgcacagtcttagctatgctttgacatctgcgggccttgaggtgcgcgttgtctctgccgggcttgactgtaaggctgtatttccatccggggttgcaacccccaccacccctggtgaggtctccgctttttgttcagctttgtacaggttcaaccgttgtgttcagcggcactcccttattgggggtttgtggtattaccctgaggggctaattggcttgtttccgccatttgctcccggccatacctgggagtcagccaatcccttctgtggagagagcaccctctatactcgcacctggtcggtgtcgggcttctctagttgtttttctccacccgagccacccgctccagaactatcatctcctgttgaggttgacacacctgtggctgttggcgttccatccccagccacttcggcagtaccccaaccttcagtccctgaacaggccgcgccattgccggattcggttgacggcggtgctgctccggccccattgagcacctctactacacccccggtgccagcgcagcacgtaacccacccatctggctcccgtcggcggttacttcacacctaccctgatggctcaaaagtgtatgctggctccctttttgaatctgagtgtacttggttagttaatgcatccaatcctggtcaccgccccggtggcggcctttgccacgcattttaccaacggttcccggagtcatttgaccctgctgagtttattatgtctgacgggtttgcggcctataccttgacaccccgacccattatccatgctgttgcccctgattatcgggtggaacataaccctaagaggctcgaggctgcctatcgagagacgtgttcccgtcgtgggacggctgcttatcccttgcttggcgccggtatatacagggtgcccgttgggctgagctttgatgcttgggagcgtaaccaccgacccggggatgagttgtacctgactgagccggctatagcttggttcgagaccaaccgacctactctccccgcgcttactattactgaggacacagcacgaacagcgaacctggctcttgagttagattcagccactgaggtcggcagggcgtgtgccggctgtcgtgtcgagccgggtgttgtccattaccagttcacagcaggtgtccctggctccggcaagtcacggtcaattcagcagggcgaggtagacgtcgtagtggtgccaacccgtgagttgcgtaactcgtggcggcgccgcgggtttgcagcttatacaccacacaccgcggcccgtgttacctgtggccgtagggttgttattgacgaggccccgtcgcttccaccacatttacttttgctgcatatgcagcgagcctcgtcggttcatctccttggcgaccccaaccaaatccctgctatcgattttgagcatgctggcctcgttccggcgatccggcctgagcttgttccaacaaagtggtggcaccttactcataggtgtcctgcggatgtttgtgagctaatccgcggcgcgtacccaaagatccagacggcaagccgagttctccgctccctgttctggggggagccccccgttggccaaaaactagtgtttactcaggcggcgaaggccgccaaccccggtgcaattacagtccacgaggcccagggtgctacatttactgagactactattattgcaacggctgatgcccgcgggctgattcagtcctccagggcccacgctattgtggccctgactcgccacacagagaaatgtgtggtcgttgacgccccggggcttcttcgtgaggttggcatttcagatgccattgttaataacttttttctttctggcggccaggttggtcagcaccgcccatcagtcatacctcgtggcactgttgatagcaatgttgccacgcttgatgcatttccaccttcttgtcaatttagcgcctatcaccagcttgcagaggagcttggtcatcggccggccccgattgccgccgttctgcccccttgtccggagctcgagcaaggcttactctatatgccccaggaactgaccacgtccgatagtgtgctcacattcgaacttacagacattgtacactgtcgtatggcggcacctagtcagcgcaaagcagtcctgtcgacccttgtcggcaggtacggccgccgtacgaagttgtatgaagctgctcacacggatgttcgtgggtccctctgtcattttctccccgagcttggccccatcagcgttactacctgcgagctttatgagcttgttgaggctatggtggagaagggtcaggatggctctgcagtcttggaactcgatctatgcagtcgtgatgtgtcacgtataacattcttccagaaggactgtaataaattcacaacaggcgagacaatagcgcacggtaaagttgggcaggggatatctgcgtggagcaagaccttttgtgccctgtttggcccatggttccgcgctattgagaaggagattcttgcggcgcttgcccctaatgtattttatggtgatgcatatgaagatacagttttggccgccgctgtcgccggggccccaggctgtaaggtttttgagaatgatttctctgagtttgacagcacccaaaataacttttcactcggactggagtgcataatcatggaggaatgtggcatgccgcagtggatgatccgactttatcatcttgttcgctccgcctgggtcctgcaggctcccaaggagtccctgcgagggttctggaagaagcactcgggcgagcctggcactctgctctggaacactgtctggaacatggcggttattgcgcattgttatgaattccgtgacctaaaggtcgcggcatttaaaggagatgactcagttgtgctctgtagtgactaccggcagagccgcaacgctgctgctctaatcgccggttgcgggttgaaacttaaggtggattttaggcctattgggctatatgctggtgttgttgtggccccgggcctcgggaccctccctgatgtcgttaggtttgctgggcggctctcagagaaaaactgggggccgggttcagagagggcggaacagctacggctggccgtttgtgattttctgcgaaagttaacgaatgtggctcaggtctgtgtggatgttgtttcgcaggtttatggtgtcagccccggtttagtacataacctgattggaatgctccagaccattgctgatggtaaggcgcattttactgaaacagtcaaacccgtccttgatcttactagctctattatataccgggtggaatgaataacatgttcttttgctctgtgcatggagatgccaccatgcgctctcgggctcttctgtttctgctcttcgtgcttctgcctatgctgcccgcgccaccggccggtcagccgtctggccgtcgccgcgggcggcgcagcggcggtgccggcggtggtttctggggtgaccgggttgattctcagcccttcgccctcccctatattcatccaaccaaccccttcgcatctgacataccaaccgcagccgggtctggagctcgccctcggcagccggcccgtccactcggctccgcttggcgtgaccagtcccagcgccccgccgctcccgcccgccgtcgatctgccccagctggggcttcgccgctgactgctgtggccccggcccctgataccgcccctgtgcccgacgttgactcccgcggcgctatactacgccgccagtataatctatcaacatccccgcttacgtctaccatcgctactggcaccaatcttgttttatatgctgctccccttagccctctgcttccgcttcaagatgggactaacactcatattatggccactgaagcatcaaattacgcccagtaccgcgttgtccgtgccaccatccggtatcgcccacttgtgccgaatgctgttggcgggtacgctatatctatttctttctggccccagactacaactaccccaacatctgtcgatatgaattctattacctctactgatgttcggattcttgttcagcctggtatcgcctccgagcttgtgattcccagtgagcgcctgcactaccgcaatcagggctggcgctcggttgagacctctggtgtcgcggaggaggaggcgacctccggccttgttatgctctgcatccatggatcacctgtgaactcttacactaatacaccttacactggtgctcttggcttactcgatttcgcacttgaacttgagttccgtaatttgacacccggtaacacgaatacgcgcgtttcccgttattcgagtagcgcgcgccacaagctacgtcgtgggcccgacggtactgctgagctaaccaccactgctgctacacggtttatgaaagatcttcacttcacagggaccaatggtgttggtgaggtcggtcgcggtatagcgctaactctgtttaatcttgctgacacgcttctcggcgggctcccgacagaattgatttcgtcggctggtggccagctattttactctcgccccgtcgtctcagccaatggcgagccgacagtgaagctttacacttcagtcgagaacgcccagcaggataagggtatagctattccacatgatatcgaccttggtgagtctcgagttgtcatccaggattacgataaccagcacgagcaggaccgtcccaccccgtctccagccccttcccgccctttttctgtcctccgcgctaatgatgtgctttggctctcacttacagctgctgagtatgatcagactacttatggttcttccactaatcctatgtatgtctctgatactgtgacatttgtcaatgtggcgactggtgctcagggggtctctcgctctctggactggtctaaagtcacccttgatgggcgtccacttactactatccagcagtactctaagaccttctttgttctgcccctccgtggcaagctctctttctgggaggctggcaccactaaggccggctacccttataattataataccactgccagtgaccagatcttgattgaaaatgcagccggtcaccgtgtttgtatctcaacctacactactaatcttggctctggccctgtttctatttcttctgtcggtgtccttgcacctcattctgcgctggccgctttagaggacactgttgattatcctgctcgcgcccacacctttgatgatttctgccctgagtgccgtacgctcggccttcagggctgtgcttttcaatcaactgttgctgagctgcagcgtcttaaaatgaaggtgggtaaaacccgggagtattgatttattgtgcttgtaccttccttctgatttgtttcttttatttccttcttctgcgtttcgcgctccctggaaaaaaaaaa

SEQ ID NO: 12 - protein, 1707 aa, Hepatitis E virus JSN-FH ORF1
  MISC_FEATURE (281)..(281): X aa is any amino acid
MEAHQFIKAPGVTTAIEQAALAAANSALANAVVVRPFLSRLQTEILINLMQPRQLVFRPEVLWNHPIQRVIHNELEQYCRARAGRCLEVGAHPRSINDNPNVLHRCFLKPVGRDVQRWYTAPTRGPAANCRRSALRGLPPVDRTYCFDGFSGCTFAAETGIALYSLHDLWPADVAEAMARHGMTRLYAALHLPPEVLLPPGTYHTTSYLLIHDGDRAVITYEGDSSAGYNHDVSILRAWIRTTKVTGDHPLVIERVRAVGCHFVLLLTAAPEPSPMPYVPXPRSTEVYVRSIFGPGGSPSLFPTACSTKSTFHAVPVHIWDRLMLFGATLDDQAFCCSRLMTYLRGISYKVTVGALVANEGWNASEDALTAVITAAYLTICHQRYLRTQAISKGMKRLELEHAQKFITRLYSWLFEKSGRDYIPGRQLQFYAQCRRWLSAGFHLDPRVLVFDEAAPCRCRSFLRKAAHRFCCFMRWLGQDCTCFLQPVEGRVGEQGYDNEAFEGSDVDPAEEATVNISGSYIVTGSQLQPLYQALGIPSDLAARAGRLTATVEVLDADGRLTCKTTIGNKTFTTVFTDGAQLEANGPEQYVLSFDSAKQTMAAGPHSLSYALTSAGLEVRVVSAGLDCKAVFPSGVATPTTPGEVSAFCSALYRFNRCVQRHSLIGGLWYYPEGLIGLFPPFAPGHTWESANPFCGESTLYTRTWSVSGFSSCFSPPEPPAPELSSPVEVDTPVAVGVPSPATSAVPQPSVPEQAAPLPDSVDGGAAPAPLSTSTTPPVPAQHVTHPSGSRRRLLHTYPDGSKVYAGSLFESECTWLVNASNPGHRPGGGLCHAFYQRFPESFDPAEFIMSDGFAAYTLTPRPIIHAVAPDYRVEHNPKRLEAAYRETCSRRGTAAYPLLGAGIYRVPVGLSFDAWERNHRPGDELYLTEPAIAWFETNRPTLPALTITEDTARTANLALELDSATEVGRACAGCRVEPGVVHYQFTAGVPGSGKSRSIQQGEVDVVVVPTRELRNSWRRRGFAAYTPHTAARVTCGRRVVIDEAPSLPPHLLLLHMQRASSVHLLGDPNQIPAIDFEHAGLVPAIRPELVPTKWWHLTHRCPADVCELIRGAYPKIQTASRVLRSLFWGEPPVGQKLVFTQAAKAANPGAITVHEAQGATFTETTIIATADARGLIQSSRAHAIVALTRHTEKCVVVDAPGLLREVGISDAIVNNFFLSGGQVGQHRPSVIPRGTVDSNVATLDAFPPSCQFSAYHQLAEELGHRPAPIAAVLPPCPELEQGLLYMPQELTTSDSVLTFELTDIVHCRMAAPSQRKAVLSTLVGRYGRRTKLYEAAHTDVRGSLCHFLPELGPISVTTCELYELVEAMVEKGQDGSAVLELDLCSRDVSRITFFQKDCNKFTTGETIAHGKVGQGISAWSKTFCALFGPWFRAIEKEILAALAPNVFYGDAYEDTVLAAAVAGAPGCKVFENDFSEFDSTQNNFSLGLECIIMEECGMPQWMIRLYHLVRSAWVLQAPKESLRGFWKKHSGEPGTLLWNTVWNMAVIAHCYEFRDLKVAAFKGDDSVVLCSDYRQSRNAAALIAGCGLKLKVDFRPIGLYAGVVVAPGLGTLPDVVRFAGRLSEKNWGPGSERAEQLRLAVCDFLRKLTNVAQVCVDVVSQVYGVSPGLVHNLIGMLQTIADGKAHFTETVKPVLDLTSSIIYRVE

SEQ ID NO: 13 - protein, 211 aa, Hepatitis E virus JSN-FH ORF2
MRSRALLFLLFVLLPMLPAPPAGQPSGRRRGRRSGGAGGGFWGDRVDSQPFALPYIHPTNPFASDIPTAAGSGARPRQPARPLGSAWRDQSQRPAAPARRRSAPAGASPLTAVAPAPDTAPVPDVDSRGAILRRQYNLSTSPLTSTIATGTNLVLYAAPLSPLLPLQDGTNTHIMATEASNYAQYRVVRATIRYRPLVPNAVGGYAISISF

SEQ ID NO: 14 - DNA, 2582 bp, Hepatitis E virus Mexico (M74506)
gccatggaggcccaccagttcattaaggctcctggcatcactactgctattgagcaagcagctctagcagcggccaactccgcccttgcgaatgctgtggtggtccggcctttcctttcccatcagcaggttgagatccttataaatctcatgcaacctcggcagctggtgtttcgtcctgaggttttttggaatcacccgattcaacgtgttatacataatgagcttgagcagtattgccgtgctcgctcgggtcgctgccttgagattggagcccacccacgctccattaatgataatcctaatgtcctccatcgctgctttctccaccccgtcggccgggatgttcagcgctggtacacagccccgactaggggacctgcggcgaactgtcgccgctcggcacttcgtggtctgccaccagccgaccgcacttactgttttgatggctttgccggctgccgttttgccgccgagactggtgtggctctctattctctccatgacttgcagccggctgatgttgccgaggcgatggctcgccacggcatgacccgcctttatgcagctttccacttgcctccagaggtgctcctgcctcctggcacctaccggacatcatcctacttgctgatccacgatggtaagcgcgcggttgtcacttatgagggtgacactagcgccggttacaatcatgatgttgccaccctccgcacatggatcaggacaactaaggttgtgggtgaacaccctttggtgatcgagcgggtgcggggtattggctgtcactttgtgttgttgatcactgcggcccctgagccctccccgatgccctacgttccttacccgcgttcgacggaggtctatgtccggtctatctttgggcccggcgggtccccgtcgctgttcccgaccgcttgtgctgtcaagtccacttttcacgccgtccccacgcacatctgggaccgtctcatgctctttggggccaccctcgacgaccaggccttttgctgctccaggcttatgacgtaccttcgtggcattagctataaggtaactgtgggtgccctggtcgctaatgaaggctggaatgccaccgaggatgcgctcactgcagttattacggcggcttacctcacaatatgtcatcagcgttatttgcggacccaggcgatttctaagggcatgcgccggcttgagcttgaacatgctcagaaatttatttcacgcctctacagctggctatttgagaagtcaggtcgtgattacatcccaggccgccagctgcagttctacgctcagtgccgccgctggttatctgccgggttccatctcgacccccgcaccttagtttttgatgagtcagtgccttgtagctgccgaaccaccatccggcggatcgctggaaaattttgctgttttatgaagtggctcggtcaggagtgttcttgtttcctccagcccgccgaggggctggcgggcgaccaaggtcatgacaatgaggcctatgaaggctctgatgttgatactgctgagcctgccaccctagacattacaggctcatacatcgtggatggtcggtctctgcaaactgtctatcaagctctcgacctgccagctgacctggtagctcgcgcagcccgactgtctgctacagttactgttactgaaacctctggccgtctggattgccaaacaatgatcggcaataagacttttctcactacctttgttgatggggcacgccttgaggttaacgggcctgagcagcttaacctctcttttgacagccagcagtgtagtatggcagccggcccgttttgcctcacctatgctgccgtagatggcgggctggaagttcatttttccaccgctggcctcgagagccgtgttgttttcccccctggtaatgccccgactgccccgccgagtgaggtcaccgccttctgctcagctctttataggcacaaccggcagagccagcgccagtcggttattggtagtttgtggctgcaccctgaaggtttgctcggcctgttcccgcccttttcacccgggcatgagtggcggtctgctaacccattttgcggcgagagcacgctctacacccgcacttggtccacaattacagacacacccttaactgtcgggctaatttccggtcatttggatgctgctccccactcgggggggccacctgctactgccacaggccctgctgtaggctcgtctgactctccagaccctgacccgctacctgatgttacagatggctcacgcccctctggggcccgtccggctggccccaacccgaatggcgttccgcagcgccgcttactacacacctaccctgacggcgctaagatctatgtcggctccattttcgagtctgagtgcacctggcttgtcaacgcatctaacgccggccaccgccctggtggcgggctttgtcatgctttttttcagcgttaccctgattcgtttgacgccaccaagtttgtgatgcgtgatggtcttgccgcgtatacccttacaccccggccgatcattcatgcggtggccccggactatcgattggaaca

SEQ ID NO: 15 - DNA, 255 bp, Hepatitis E virus Japan JRA1 (AP003430)
ggcagaccacgtatgtggtcgatgccatggaggcccaccagttcattaaggctcctggcattactactgccatagagcaggctgctctggctgcggccaactccgccttggcgaatgctgtggtggttcggccgtttttgtctcgcgtacaaaccgagatccttattaatttgatgcaaccccggcagttggttttccgccctgaggtgctttggaaccatcctattcagagggttatacacaatgaattagaac

SEQ ID NO: 16 - DNA, 254 bp, Hepatitis E virus Japan JKN-Sap (AB074981)
gcagaccacgtatgtggtcgatgccatggaggcccatcagttcattaaggctcctggcattactactgccattgagcaggctgctctggctgcggccaattccgccttggcgaatgctgtggtggtccggccgttcttatctcgtgtacaaactgagattcttattaatttgatgcaaccccggcagttggttttccgccctgaggtgctctggaatcaccctatccagcgggttatacataatgaattggaac

SEQ ID NO: 17 - DNA, 238 bp, Hepatitis E virus JMY-Haw (AB074920)
gtcgatgccatggaggcccatcaattcattaaggctcctggcattactactgccattgagcaggctgctctggctgcggccaattccgccttggcgaatgctgtggtggtccggccgtttttatctcgtgtacaaaccgagattcttattaatttgatgcaaccccggcagttggttttccgtcctgaggtgctctggaatcatcctattcagcgggttatacataatgaattggaac

SEQ ID NO: 18 - DNA, 264 bp, Hepatitis E virus  USA US-1 (AF060669)
tcgacagggggcagaccacgtatgtggtcgatgccatggaggcccatcagttcattaaggctcctggcattactactgctattgagcaggctgctctggctgcggctaattccgccttggcgaatgctgtggtggttcggccgtttctttctcgtgtgcaaactgagattcttattaatttgatgcaaccccggcagttggtcttccgccctgaggtgctttggaatcatcctatccagcgggttatacataatgaattagagc

SEQ ID NO: 19 - DNA, 238 bp, Hepatitis E virus SWINE HEV (AF082843)
ttcgatgccatggaggcccatcagttcattaaggctcctggcattactactgccattgagcaggctgctctggctgcggccaactccgccttggcgaatgctgtggtggttcggccgtttttatctcgtgtacaaactgagatccttattaatttgatgcaaccccggcagttggttttccgccctgaggtactttggaatcatcctatccagcgggcaatacataatgaactggaac

SEQ ID NO: 20 - DNA, 238 bp, Hepatitis E virus Japan JAK-Sai (AB074915)
gtcgacgccatggaggcccatcagtttataaaggctcctggcgtcactactgctattgagcaggcagctctagcagcggccaactccgccttggcgaatgctgtggtggttcggcctttcttatcccggctacagacagagatactcattaacctgatgcagccccggcagcttgttttccggcctgaggttttatggaatcacccaattcagcgtgtgatccacaatgagctcgaac

SEQ ID NO: 21 - DNA, 238 bp, Hepatitis E virus Japan JKK-Sap (AB074917)
gtcgacgccatggaggcccaccagttcataaaggctcctggcgtcactactgctattgagcaggcagctctagcagcggccaactccgccttggcgaatgctgtggtggttcggcctttcctatcccggctacagacagagatacttattaacctgatgcagccccggcagcttgttttccggcctgaagtcttgtggaaccacccaattcagcgcgtgatccacaacgagcttgagc

SEQ ID NO: 22 - DNA, 238 bp, Hepatitis E virus Japan JSN-FH
gtcgacgccatggaggcccatcagttcataaaggctcctggcgtcacaactgctattgagcaggcagctctagcagcggccaactccgccttggcgaatgctgtggtggttcggccttttctgtcccggctacagacagagatacttattaacttgatgcagccccggcagcttgttttccggcctgaggtcctgtggaatcaccccattcaacgcgtgatccacaatgagcttgaac

SEQ ID NO: 23 - DNA, 194 bp, Hepatitis E virus China type4 (AJ272108)
cactactgctattgagcaggcagctctagcagcggccaactccgccctggcgaatgctgtggtggttcggcctttcttgtcccggcttcagactgagattctcataaatttgatgcagccttggcagcttgttttccggcctgaggtcctgtggaatcacccaatccagcgtgtgatccacaatgagcttgagc

SEQ ID NO: 24 - DNA, 256 bp, Hepatitis E virus Burma B1 (M73218)
aggcagaccacatatgtggtcgatgccatggaggcccatcagtttattaaggctcctggcatcactactgctattgagcaggctgctctagcagcggccaactctgccctggcgaatgctgtggtagttaggccttttctctctcaccagcagattgagatcctcattaacctaatgcaacctcgccagcttgttttccgccccgaggttttctggaatcatcccatccagcgtgtcatccataacgagctggagc

SEQ ID NO: 25 - DNA, 256 bp, Hepatitis E virus China Uigh (D11093)
aggcagaccacatatgtggtcgatgccatggaggcccatcagtttatcaaggctcctggcatcactactgctattgagcaggctgctctagcagcggccaattctgcccttgcgaatgctgtggtagttaggccttttctctctcaccagcagattgagatccttattaacctaatgcaacctcgccagcttgttttccgccccgaggttttctggaaccaccccatccagcgtgtcatccataatgagctggagc

SEQ ID NO: 26 - DNA, 256 bp, Hepatitis E virus China Hebei (M94177)
aggcagaccacatatgtggtcgatgccatggaggcccatcagtttattaaggctcctggcatcactactgctattgagcaggctgctctagcagcggccaactctgcccttgcgaatgctgtggtagttaggccttttctctctcaccagcagattgagatccttattaacctaatgcaacctcgccagcttgttttccgccccgaggttttctggaaccatcccatccagcgtgttatccataatgagctggagc

SEQ ID NO: 27 - DNA, 256 bp, Hepatitis E virus China Xinjiang (D11092)
aggcagaccacatatgtggtcgatgccatggaggcccatcagtttatcaaggctcctggcatcactactgctattgagcaggctgctctagcagcggccaactctgcccttgcgaatgctgtggtagttaggccttttctctctcaccagcagattgagatccttattaacctaatgcaaccccgccagcttgttttccgccccgaggttttctggaaccatcccatccagcgtgttatccataatgagctggagc

SEQ ID NO: 28 - DNA, 256 bp, Hepatitis E virus Nepali (AF051830)
aggcagaccacatatgtggtcgatgccatggaggcccatcagtttattaaggctcctggcatcactactgctattgagcaggctgctctagcagcggccaactctgccctggcgaatgctgtggtagttaggccttttctctctcaccagcagattgagattctcattaacctaatgcaacctcgccagcttgttttccgccccgaggttttctggaatcatcccatccagcgtgtcatccataacgagctggagc

SEQ ID NO: 29 - DNA, 255 bp, Hepatitis E virus India FH strain (X98292)
ggcagaccacctatgtggtcgatgccatggaggcccatcagtttatcaaggctcctggcatcactactgctattgagcaggctgctctggcagcggccaactctgccctggcgaatgctgtggtagttaggccctttctctctcaccagcagattgagatcctcattaacctaatgcaacctcgccagcttgttttccgccccgaggttttctggaaccaccccatccagcgtgttatccacaatgagttggagc

SEQ ID NO: 30 - DNA, 226 bp, Hepatitis E virus Pakistan SAR55 (M80581)
gaggcccatcagtttatcaaggctcctggcatcactactgctattgagcaggctgctctagcagcggccaactctgcccttgcgaatgctgtggtagttaggccttttctctctcaccagcagattgagatccttattaacctaatgcaacctcgccagcttgttttccgccccgaggttttctggaaccatcccatccagcgtgttatccataatgagctggagc

SEQ ID NO: 31 - DNA, 232 bp, Hepatitis E virus Mexico (M74506)
gccatggaggcccaccagttcattaaggctcctggcatcactactgctattgagcaagcagctctagcagcggccaactccgcccttgcgaatgctgtggtggtccggcctttcctttcccatcagcaggttgagatccttataaatctcatgcaacctcggcagctggtgtttcgtcctgaggttttttggaatcacccgattcaacgtgttatacataatgagcttgagc

SEQ ID NO: 32 - DNA, 20 bp, Artificial
  Synthetic DNA
gcagaccacrtatgtgktcg

SEQ ID NO: 33 - DNA, 20 bp, Artificial
  Synthetic DNA
ccacrtatgtggtcgaygcc

SEQ ID NO: 34 - DNA, 20 bp, Artificial
  Synthetic DNA
acmarctgscgrggytgcat

SEQ ID NO: 35 - DNA, 20 bp, Artificial
  Synthetic DNA
cgytgratwggrtgrttcca

SEQ ID NO: 36 - DNA, 19 bp, Artificial
  Synthetic DNA
tgktcgaygccatggaggc

SEQ ID NO: 37 - DNA, 19 bp, Artificial
  Synthetic DNA
tgktcgaygccatggaggc

SEQ ID NO: 38 - DNA, 20 bp, Artificial
  Synthetic DNA
aygccatggaggcccaycag

SEQ ID NO: 39 - DNA, 20 bp, Artificial
  Synthetic DNA
ckracyaccacagcattcgc

SEQ ID NO: 40 - DNA, 20 bp, Artificial
  Synthetic DNA
ggcckracyaccacagcatt

SEQ ID NO: 41 - DNA, 53 bp, Artificial
  Synthetic DNA
aaggctcctggcrtyactactgcyatwgagcaggcwgctctrgcwgcggccaa

SEQ ID NO: 42 - DNA, 19 bp, Artificial
  Synthetic DNA
ctcctggcatcactactgc

SEQ ID NO: 43 - DNA, 19 bp, Artificial
  Synthetic DNA
ctcctggcattactactgc

SEQ ID NO: 44 - DNA, 7256 bp, Hepatitis E virus Japan JKN-Sap (AB074918)
gcagaccacgtatgtggtcgatgccatggaggcccatcagttcattaaggctcctggcattactactgccattgagcaggctgctctggctgcggccaattccgccttggcgaatgctgtggtggtccggccgttcttatctcgtgtacaaactgagattcttattaatttgatgcaaccccggcagttggttttccgccctgaggtgctctggaatcaccctatccagcgggttatacataatgaattggaacagtactgccgggcccgggccggccgttgcctggaggttggggctcacccgaggtccattaatgacaatcccaatgtcctgcacaggtgctttcttagaccggttggccgagacgtccagcgctggtactctgcccccacccgtggccctgcggccaactgccgccgctccgcgttgcgtggtctccctcccgctgaccgcacttattgctttgatggattctcccgctgtgcttttgctgcagagaccggcgtggccctttactctctgcatgacctttggccagctgatgtcgcagaggctatggcccgccacgggatgacacgcctatatgctgcactacacctccctcctgaggtgctgttgccacccggcacttaccacacaacctcgtatctcctgatccacgacggcgaccgtgccgtcgtaacttatgagggcgatactagtgcaggctacaatcacgatgtttccatacttcgtgcgtggatccgtactactaaaatagttggtgaccacccgttggttatagagcgtgtgcgggccattggatgtcattttgtgctgctgctcaccgcagcccctgagccgtcacctatgccttatgtcccctaccctcgttcaactgaggtgtatgtacgatctatatttggccctggtggctccccatctttgttcccgtcagcctgctctactaaatctacttttcatgctgtcccggttcatatctgggaccggcttatgctttttggtgccaccctggacgatcaggcgttttgttgttcacggctcatgacttacctccgtggtattagctacaaggtcactgtcggtgcgcttgttgctaatgagggatggaatgcctctgaggacgcccttactgcagtgatcactgcggcttacctgactatttgccaccaacgctaccttcgaacccaggcgatatccaagggtatgcgccggttggaggttgagcatgcccagaaattcatcacaaggctctacagctggctatttgagaaatctggtcgtgattatatccccggccgccagcttcagttctatgcacaatgtcggcggtggttatctgcaggcttccacctcgaccccagggtgcttgtcttcgatgaagcagtgccatgccgctgtaggacgtttttgaagaaggtcgcgggtaaattctgctgttttatgcggtggctagggcaggagtgcacctgtttcttggagccagctgagggcctaattggagaccaaggccatgataatgaggcctatgagggttctgaggtcgacccggctgaacctgcacatcttgatgtttcggggacctatgctgtccatgggcatcagcttgaggccctttatagggcactcaatgtcccacatgatattgccgctcgagcctcccggctaacggctactgtcgagcttgttgcaagtccagaccgcttagagtgccgtactgtgcttggtaataagacctttcggacaacggtggttgatggtgcccatcttgaagcaaatggccctgaggagtatgttctatcattcgacgcctctcgtcagtctatgggggccggatcgcacagcctcacatatgagctcacccctgctggtctgcaggtcaggatttcatctaatggcttggattgtaccgccgtattccctcccggcggcgcccctagcgccgcaccgggggaggtggcagccttctgcagcgccctttatagatataacaggttcacccaacggcactcgctaaccggtggattatggttacaccctgaggggttgctgggcatcttcccccctttctctcctggacacatctgggagtctgctaacccattttgtggggaggggaccttgtatacccgaacctggtcaacatctggcttctctagcgacttctccccccctgaggcggccgcccctgttccggctgctgccccggggctgccccaccccaccccacctgttagtgacatttgggtgctgccaccaccctcagaggagtcccagatcgatgcggcacctgtgccccctgtccctaagactgttggattgcctagccccattgtacttgctcctccctcccctcttccttcccccgtgcgtaagccaccatcacccccgccttctcgcactcgtcgtctcctctacacctatcctgacggcgctagggtatatgcggggtcgttgtttgaatcagactgtgactggctagttaacgcctcaaatccgggccaccgtcctggaggtggcctctgccacgccttttaccaacgcttcccagaggcgttttacccaactgaattcattatgcgtgagggccttgcagcatataccctgaccccgcgccctatcattcatgcagtggctcccgactatagggtcgagcagaatccgaagaggcttgaggcagcgtaccgggaaacttgctcccgtcgcggcaccgctgcctatccgcttttgggctcgggtatataccaggtccctgttagtctcagttttgatgcctgggaacgcaatcatcgccccggcgacgagctttacttgactgagcccgctgcagcttggtttgaggctaataagccatcgcagccggcgcttactataactgaggacacggctcgtacggccaatctggcattagagattgatgccgccacagaggttggccgtgcttgtgccggctgcactatcagcccgggggttgtgcattaccagtttactgccggggtcccgggctcgggcaagtcaaggtccatacaacagggagacgtcgatgtggtggttgtgcccacccgggagcttcgtaatagttggcgccgccggggttttgcggctttcacacctcacacagcggcccgtgttactattggtcgccgcgttgtgattgatgaggctccgtccctcccgccgcacttgctgctgctacacatgcaacgggcctcctcggtccatctcctcggcgacccaaatcagattcctgctattgattttgaacatgccggcctggtccccgcgatccgtcccgagcttgcaccaacgagctggtggcatgttacacaccgctgcccggcagatgtgtgtgagcttatacgtggggcctaccctaagatccagaccacgagtcgtgtgctacggtccctgttttggaacgaaccggccattggccagaagctggttttcacgcaggctgctaaggctgctaatcctggtgcgatcacggttcatgaggctcagggtgccaccttcacggagaccacaatcatagccacggctgatgctaggggccttattcagtcatcccgagctcacgctatagtcgcactcacccgccacactgagaagtgtgttattttagatgcccccggcctactgcgcgaggtcggtatttcagatgtgattgtcaataactttttccttgctggtggagaggttggccaccaccgcccctccgtgatacctcgcggtaaccccgatcagaatctcgggactctacaggcattcccgccgtcttgccagattagtgcctaccaccagttggctgaggaattaggccaccgcccagctcctgtcgccgccgtcttacccccttgcccggagcttgagcagggcctgctctacatgccacaagagcttactgtgtccgatagtgtgttggtatttgaactcacagatatagtccattgccgtatggccgctccaagccagcgaaaggctgttctctcaacacttgtcgggaggtatggccgtagaacgaaattatatgaggcggcacattcagatgttcgtgagtccctagctaggttcatccccactatcgggcctgttcaggccaccacatgtgagttgtatgagttggttgaggccatggtggagaagggtcaggacggctctgccgtcctagagcttgacctttgcaatcgtgacgtctcgcgtatcacatttttccaaaaggattgcaataaattcacaactggtgagactattgcccatggcaaggttggccagggtatatcggcctggagtaagaccttctgtgccctgtttggcccgtggttccgcgctatagaaaaagagatattggccctgctcccgcctaatatcttttatggcgacgcttatgaagagtcagtgtttgctgccgctgtgtctggggcggggtcatgtatggtatttgaaaatgatttttcggaatttgacagtactcagaacaacttctctctcggccttgagtgtgtggtcatggaggagtgcggcatgccccagtggttgattaggttgtaccatctggttcggtcagcctggattttgcaggcgccgaaggagtctcttaagggtttttggaagaagcactctggtgagcctggtacccttctctggaacaccgtctggaacatggcgattatagcgcactgttacgagttccgtgactttcgcgttgccgccttcaagggtgatgattcggtggtcctttgcagcgactatcggcagagccgcaatgcggctgccttaattgcaggctgtgggctcaaattgaaggtcgattatcgtcctattgggctgtatgctggggtggtggtggcccctggtttggggacactgcccgacgtggtgcgttttgctggtcggttgtctgaaaagaattggggccccggccctgaacgtgctgagcagctgcgtctcgctgtttgtgatttccttcgagggttgacgaatgttgcgcaggtttgtgttgatgttgtgtcccgtgtttacggagtcagccccgggctggtacataaccttattggcatgctgcagaccattgccgatggcaaggctcacttcacagagaccattaaacctgtgcttgaccttacgaattccatcatacagcgggaagaatgaataacatgtcttttgcatcgcccatgggatcaccatgcgccctagggctgttctgttgttgttcctcgtgcttttgcctatgctgcccgcgccaccggccggtcagccgtctggccgtcgccgtgggcggcgcagcggcggtgccggcggtggtttctggggtgacagggttgattctcagcccttcgccctcccctatattcatccaaccaaccccttcgccgccgatgtcgtttcacaacccggggctggaactcgccctcgacagccgccccgcccccttggctccgcttggcgtgaccagtcccagcgcccctccgctgccccccgccgtcgatctgccccagctggggctgcgccgttgaccgctgtatcaccggctcctgacacagcccctgtgcctgatgttgattcacgcggtgctatcctgcgccggcagtacaatctgtccacgtccccgctcacgtcatctgtcgcctcgggcacaaatctggttctctatgctgccccgcttaatcctctcctgccccttcaggatggcaccaacactcatattatggccactgaggcatccaattatgcccagtatcgggttgttcgagctacgatccgttatcgcccgttggtgccgaatgcagttggcggttatgctatttctatttctttttggcctcaaaccacaactactcccacctctgtcgacatgaattctatcacttccactgatgttaggattttggttcagcccggcattgcctccgagttagtcatccctagtgagcgcctccactaccgcaatcaaggctggcgctctgttgagaccacgggtgtggccgaggaggaggctacttccggtctggtaatgctttgtattcatggctctcctgttaattcctacactaatacaccttatactggtgcactggggctccttgattttgcattagagcttgaatttagaaatctgacacccgggaacacaaacacccgtgtttcccggtataccagcacagcccgtcaccggctgcgccgcggtgctgatgggactgctgagcttaccaccacagcagccacacgtttcatgaaggatttacattttactggcacgaatggtgttggtgaggtgggtcgtggcatcgctctgacattgtttaatctcgctgacacgcttctcggtggtttaccgacagaattgatttcgtcggccgggggtcaactgttttactcccgccctgttgtctcggccaatggcgagccaacagtaaagttatacacatctgttgagaatgcgcagcaagataagggcattaccatcccacacgatatagatctgggtgactcccgtgtggttattcaggattatgataaccagcacgagcaagatcggcctactccgtcacctgccccctcccgccctttctcagttcttcgtgctaatgatgttctgtggctctccctcaccgccgctgagtatgaccagactacgtatgggtcgtccaccaaccctatgtatgtctccgacacagtcacgctcgttaatgtggccactggagcccaggctgtggcccgctctcttgattggtctaaagttaccttggatggccgcccccttactaccattcagcagtattctaagacattctatgtccttccgctccgcgggaagctgtctttctgggaggctggtacgactaaggccggttacccgtataattataatactactgctagtgatcagatcttgattgagaacgcggccggccaccgtgtcgctatttctacctatactactagcttgggtgccggccctacctcgatctctgcggtcggtgtactagctccacattcggccctcgccgttctagaggacaccgttgattaccccgcccgcgctcacacttttgatgatttctgcccggagtgccgtaccctcggtttgcagggttgtgcattccagtctactatcgctgagcttcagcgtcttaaaatgaaggtaggtaaaacccgggagtcttaattaattccttttgtgcccccttcgcagctttctctggctttatttcttatttctgcttttcgcgctccctggaaaaaaaaaaaa

SEQ ID NO: 45 - DNA, 7233 bp, Hepatitis E virus Japan JMY-Haw (AB074920)
ccatggaggcccatcaattcattaaggctcctggcattactactgccattgagcaggctgctctggctgcggccaattccgccttggcgaatgctgtggtggtccggccgtttttatctcgtgtacaaaccgagattcttattaatttgatgcaaccccggcagttggttttccgtcctgaggtgctctggaatcatcctattcagcgggttatacataatgaattggaacagtactgccgggcccgggccggccgctgcctggaggttggggcccacccgagatccattaatgataatcccaatgtcctgcacaggtgctttcttagaccggtcggccgagatgtccaacgctggtactctgcccccacccgcggtcctgcggccaattgccgccgctccgcgttgcgtggtctccctcccgctgaccgcacttattgctttgatggattctcccgctgtgcttttgctgcagagaccggcgtggctctttactctttgcatgacctttggccagctgatgttgcagaggctatggcccgccatgggatgacacgcctatatgctgtactacaccttcctcctgaggtgctgctaccacccggcacttaccacacaacttcgtatctcctgatccacgacggcgaccgtgccgttgtaacttatgagggtgatactagtgcgggctacaaccacgatgtttccatacttcgtgcgtggatccgcactactaaaatagttggtgaccacccgctggttatagagcgtgtgcgggccattggatgtcactttgtgctgctgctcaccgcagcccctgagccgtcacctatgccttatgtcccctaccctcgttcaactgaggtgtatgtacgatctatatttggccctggtggctccccatctctgttcccgtcagcctgctctactaaatctacttttcatgctgtcccggttcatatctgggatcggcttatgctttttggtgccaccctggacgatcaggcgttttgttgttcacggctcatgacttacctccgtggtattagctacaaggtcactgtcggcgcgctggtcgctaatgaaggatggaacgcctctgaggacgcccttactgcagtgatcactgcggcttatctgactatttgccaccagcgctacctccgaacccaggcgatatccaagggtatgcgccggttggaggttgagcatgcccagaaatttattacaaggctctacagctggctatttgagaaatctggccgtgattatattcccggccgccagcttcagttctatgcacaatgccggcggtggctatctgcaggtttccatctcgaccccagggtgcttgtctttgatgaatcagtgccatgccgttgtaggacgtttttgaagaaggtcgcgggtaaattctgctgttttatgcggtggctggggcaggagtgcacctgttttttggagcccgccgagggtttagttggagaccaaggccatgacaacgaggcctatgagggttctgaggtcgacccggccgaacctgcacatcttgatgtttcgggtacctatgctgtccatgggcaccagcttgaggccctttatagagcacttaatgtcccacatgatattgccgctcgagcctcccggctaacggctactgtcgagcttgttgcaagtccagaccgcttggagtgccgtactgtgctcggtaataagaccttccggacaacggtggttgatggcgcccatcttgaagcaaatggccctgagcagtatgttctatcatttgacgcctcccgtcagtctatgggggccgggtcgcatagcctcacttatgagctcacccctgccggtctgcaggtcaggatttcatctaatggcctggattgcaccgctgtattccctcccggcggcgcccctagcgccgcaccgggggaggtggcagccttctgcagcgccctttatagatataacaggttcacccaacggcactcgctgactggtggattatggttacaccctgaggggttgctgggtatcttcccccctttctcacctgggcacatctgggagtctgctaatccattttgtggggaggggaccttgtatacccgaacctggtcaacatctggcttttctagtgacttctccccccctgaggcggccgcccctgttccggctgctgccccgggattgccccaccctactccacctgttagtgatatttgggtgctgccacccccctcagaggggtcccagatcgatgcggcacctgtgccccctgttcctaagactgttggattgcctagccccattgtacttgctcctccccccccctttccttcccccgtgcgtaagccatcatcacccccgccttctcgcacccgtcgtctcctctacacctatcctgacggcgctagggtatatgcggggtcattgtttgaatcagattgtgactggctggttaatgcctcaaacccgggacaccgtcctggaggtggcctttgccatgccttttaccagcgtttcccagaggcgttttacccgactgaattcattatgcgtgagggcctcgcagcatataccctgaccccgcgccctatcattcatgcagtggctcccgactatagggttgagcagaacccgaagaggcttgaggcagcgtatcgggaaacttgctctcgtcgtggcaccgctgcctatccgcttttgggctcgggtatataccaggtccctgttagtctcagttttgatgcctgggaacgcaatcatcgccccggcgacgagctttatctgactgagcccgctgcagcctggtttgaggctaataagccctcgcagccggcgcttactataactgaggacacggctcgtacggccaacctggcattagagattgatgccgccacagaggttggccgtgcttgtgccggctgcactattagcccagggattgtgcattatcagtttactgccggggtcccgggctcgggcaagtcaaggtccatacaacagggagacgtcgatgtggtggttgtgcccacccgggagcttcgtaatagttggcgtcgccggggttttgcggctttcacgcctcacacagcggcccgtgttactatcggtcgccgcgttgtgattgatgaggccccatccctcccgccgcatctgctgctgttacacatgcaacgggcctcctcggtccatctcctcggcgacccaaatcagattcctgctattgattttgagcatgccggcttggtccccgcgatccgccccgagcttgcgccaacgagctggtggcacgttacacaccgctgcccggcagacgtgtgcgagcttatacgtggagcctaccctaagatccagaccacgagccgtgtgctacggtctctgttttggaacgaaccggccattggccagaagctggttttcacgcaggctgctaaggctgctaatcctggtgcgatcacggttcatgaagctcagggtgccaccttcacggagaccacaattatagccacggctgacgccaggggccttattcagtcatcccgggctcacgctatagttgcactcactcgccacactgagaagtgtgttattttagatgcccccggcctgttgcgcgaggtcggtatttcggatgtgattgttaacaactttttccttgctggtggagaggtcggccaccaccgcccctctgtgataccccgcggcaaccccgatcagaatctcgggactttacaggcattcccgccgtcttgccagattagtgcctaccaccagttggctgaggaattaggccaccgcccggcccctgtcgctgccgtcttacccccttgcccggagcttgagcagggcctactctacatgccacaagagcttactgtgtccgatagtgtgttggtttttgagctcacagatatagtccactgtcgtatggccgccccaagtcagcgaaaggctgttctctctacacttgtggggaggtatggccgtagaacgaaattatatgaggcggcgcattcagatgttcgtgagtccttagctaggttcatccccactatcgggcctgttcaggccaccacatgtgagttgtatgagttggtcgaggccatggtggagaagggtcaggacggctctgccgtcctagagcttgatctttgcaatcgtgacgtctcgcgtatcacatttttccaaaaggattgcaataagtttacaactggtgagactatagcccatggcaaggttggccagggtatatcggcctggagcaagaccttctgcgccctgttcggcccgtggttccgcgctatagaaaaagaaatattggccctgctcccgcctaatattttttatggcgacgcttatgaggagtcagtgtttgctgccgctgtgtctggggcggggtcatgtatggtatttgaaaatgatttctcggagtttgacagtactcagaataatttctctctcggtcttgagtgtgtggtcatggaggagtgcggcatgccccaatggttgattaggttgtaccatctggttcggtcagcctggattttgcaggcgccgaaggagtcccttaagggtttttggaagaagcactctggtgagcccggcacccttctctggaataccgtctggaacatggcgattatagcgcattgttacgagttccgtgactttcgcgttgccgccttcaagggtgatgattcggtggtgctttgtagcgactaccggcagagccgcaatgcggctgccttaattgcaggctgtgggctcaaattgaaggtcgattaccgccctattgggctgtacgctggggtggtggtggcccctggtttagggacactgcctgatgtggtgcgttttgctggtcggttgtctgaaaagaattggggccccggccctgaacgcgctgagcagctgcgtctcgccgtttgtgatttccttcgagggttgacgaatgtcgcgcaggtctgtgttgatgttgtgtcccgtgtctacggagtcagccccgggctggtacataaccttattggcatgctgcagactatcgccgatggcaaggcccactttacagagaccattaaacctgtgcttgaccttacgaattccatcatacagcgggaggaatgaataacatgtcttttgcaccgcccatgggatcaccatgcgccctagggctgttctgttgttgttcctcgtgcttctgcctatgctgcccgcgccaccggccggccagccgtctggccgtcgtcgtgggcggcgcagcggcggtaccggcggtggtttctggggtgacagggttgattctcagcccttcgccctcccctatattcatccaaccaaccccttcgccgccgatgtcgtttcacaacccggggctggagctcgccctcgacagccgccccgcccccttggctccgcttggcgtgaccagtcccagcgcccctccgctgccccccgccgtcgatctgccccagctggggctgcgccgttgaccgctgtatcaccggctcctgacacagcccctgtacctgatgttgattcacgcggcgctattctgcgccggcagtacaatttgtccacgtccccgcttacgtcatccgttgcctcgggtacaaatctggttctttatgctgccccgcttaatcctctccttccccttcaggatggcaccaacactcatattatggccactgaggcatctaattatgcccagtatcgggttgttcgagctacgatccgttatcgcccgttggtgccgaacgctgttggcggttatgctatttctatctctttttggcctcaaaccacaactactccaacttctgttgatatgaattccattacttccactgatgttaggattttggttcagcccggtattgcctccgagttagtcatccctagtgagcgcctccactaccgcaatcaaggctggcgctctgttgagaccacgggtgtggccgaggaggaggctacttccggtctggtaatgctttgcattcatggttctcctgttaattcctatactaatacaccttatactggtgcactggggctccttgattttgcattagagcttgaatttagaaatctgacacccgggaacacaaacacccgtgtttcccggtataccagcacagcccgtcaccggctgcgacgcggtgctgatgggactgctgagctcaccaccacagcagccacacgttttatgaaggatttgcattttactggcacgaacggtgttggtgaggtgggtcgtggcatcgctctgacattgtttaatctcgctgatacgcttctcggtggtttaccgacagaattgatttcgtcggccgggggtcagctgttttactcccgccctgttgtctcagccaatggcgagccaacagtaaagttatatacatctgttgagaacgcgcagcaagataagggcattaccatcccacacgatatagatttgggtgactcccgtgtggttatccaggattatgataaccagcacgagcaagatcggcctaccccgtcacctgccccctcccgccctttctcagttcttcgtgccaatgatgttctgtggctctccctcaccgccgctgagtacgaccagactacatatgggtcgtccaccaaccctatgtatgtctccgacacggtcacgctcgttaatgtggccactggtgctcaggctgttgcccgctctctcgattggtctaaagttaccttggatggccgcccccttactactattcagcagtattctaagacattctatgtcctcccgcttcgcgggaagctgtctttttgggaggctggcactaccaaggccggctacccgtataattataatactactgctagtgatcaaattttgattgagaacgcggccggccaccgtgtcgctatttctacctatactactagtttgggtgccggtcctacctcgatctctgcggtcggtgttctagccccacactcggccctcgccgctctagaggacaccgttgattaccccgcccgcgctcacacttttgatgatttttgcccggagtgccgtaccctcggtttgcagggttgtgcattccagtctactatcgctgagcttcagcgtcttaaaatgaaggtaggtaaaacccgggagtcttaattaattccttttgtgcccccttcgtagttccctctggctttatttcttatttctgctccccgcgctccctggaaaaaaaaaaaa

SEQ ID NO: 46 - DNA, 7229 bp, Hepatitis E virus Japan JAK-Sai (AB074915)
ccatggaggcccatcagtttataaaggctcctggcgtcactactgctattgagcaggcagctctagcagcggccaactccgccttggcgaatgctgtggtggttcggcctttcttatcccggctacagacagagatactcattaacctgatgcagccccggcagcttgttttccggcctgaggttttatggaatcacccaattcagcgtgtgatccacaatgagctcgaacagtattgtcgggctcgcgctggccgctgccttgaagtgggcgctcatccgcgctccattaacgacaatcccaatgttcttcaccgctgctttttaaaacctgttggccgcgatgttcagcggtggtacaccgcccctacccgtggccctgcagcgaactgtcgccgatcggcccttcgtggccttccacctgccgaccggacgtattgctttgatggtttctccggctgtacattcgctgctgagacgggcgtagccctctactcactgcacgatctctggcccgccgatgtcgctgaggcgatggctcgccacggcatgactcggttgtatgcagccctccacctccccccggaggtgctactcccccccggcacctatcatactacctcgtatctcctcatccatgatggggaccgtgcagtgattacttatgagggggactctagcgctgggtacaaccatgatgtatccatcctgcgcgcctggatccgcactaccaaggtcactggtgaccacccgttggtaattgagcgggttcgggcggtgggttgtcactttgtgctcctcctcacagccgcgcctgaaccatcaccaatgccctatgttccatatcctcgttctaccgaggtttatgtgcggtccatttttggccctggcggttcaccttccctgttcccatctgcctgctctactaagtcaacatttcatgctgtccctgtgcatatatgggacagactcatgctctttggtgcgaccctcgacgaccaggccttctgttgctcgcggctaatgacatacctccgtggcattagttacaaggttacggtcggtgctctcgtcgccaatgagggttggaatgcctccgaagatgcattgactgccgtgattactgcggcctatctcaccatttgccaccagaggtatctccgcacgcaggctatctcgaaagggatgaaaagattggagcttgagcatgcacagaagtttataacacgcctttacagctggctgtttgaaaagtccggccgtgactacatccccggccgtcaactgcagttctacgctcagtgccgtcggtggctatctgctggtttccaccttgatcctcgtgtgctcgtctttgacgaagcagccccctgccgctgccgtagccttctccgtaaagcagcacataaattttgctgctttatgcggtggctggggcaggactgtacctgtttcctccagcccatcgaaggaagggttggtgagcagggttatgacaatgaggcatttgaggggtcggacgtcgatcctgctgaggaggctactgtgagcatttctgggtcatatatcgtcactggcagccagctgcagcccctctaccaggcgcttggcatcccttctgatcttgccgcccgagcgggtcggcttactgccaccgtcgaagtctctgatgccgacggccgccttacttgtaagaccatcatgggcaataagacttttacaacagtttttactgatggtgcccagctagaggtcaacgggccagagcaatatgtgctgtcgtttgatttggctaaacagacgatggccgctggcccgcatagccttagttatgttctgacatccgcgggccttgaggtgcgtgttgtttctgctgggcttgactgtaaggctgtttttccatctggggttgcaaccccctccgcttctggcgaggtgtcagccttctgttcagctttatacaggtttaaccgttgcgttcagcggcattctcttattgggggcttgtggtattaccctgaggggctaatcggcctgttcccaccgtttgctcccggccacacctgggagtcaaccaaccctttctgtggagaaagtaccctttatactcgcacctggtcggtgtcagggttttctagctgtttctccccacttgagccccctgcctcgggttcactaccccctgcggagatcgacccacccgtgactgttgatgccccatctccatctattttggcattgccccggccctcagtttttgaacagaccacgcccccattggatccagctggtgacgctgctgcttcggccccgccaggtgcccctggtgtgcccccggcaccagcgcggcctgtaacccacccatccgggccccgccggcggttactccatacttatcctgatggctcaaaggtgtatgccggttctctctttgagtccgagtgtacttggctggttaatgcatccaatcccggtcaccgccctggtggcggtctctgccatgcgttttaccagcggtttccggagtcatttgatcccgccgagtttgtcatgtctgatgggtttgcggcctacaccttgacaccccggcccataattcacgctgttgctcccgattatcgagtagaacataatcctaagaggctcgaggctgcctatcgggagacgtgctctcgccgcgggacggccgcttaccctctgctcggcgccggtatatataaggtacctgttgggctgagttttgatgcctgggaacgtaaccaccggcccggggatgagttgtacctgaccgagccagctatagcttggtttgaggctaaccggcctactctccctgcgcttaccattactgaggacacggcacgaacagcaaatctggcgctggagttggattcggccactgaggttggccgggcatgtgccggctgtcgtgttgagcctggtgtcgtccactaccagtttacggcaggtgtccctggctccggcaagtcacggtcaattcagcagggtgaggtcgatgtggtggtggtgccaactcgtgagctgcgtaactcctggcgacgccgtgggtttgcagcttacacaccccacaccgcggcccgcgtcacccgcggtcgcaggattgttattgacgaggccccggcacttccaccacatttacttctgctacatatgcagcgggcctcgtcagtccacctcctcggcgaccccaaccagatccctgctattgacttcgagcatgccggtcttgtcccggcaatccggcctgagcttgtcccaacaaagtggtggcatcttacccataggtgcccagcagacgtctgtgagctaattcgtggcgcgtatccaaagatccagacggcaagccgtgtcctccgctctctgttttggggagagccccctgtaggccaaaagttagtattcactcaggcggcaaaggccgctaaccctggtgcaattacagtccatgaggcccagggtgccacatttactgagaccactattattgctacggcagacgcccgcgggctgatccagtcttctagggcccacgctattgtagccctgactcgccacacagagaaatgtgtagttgttgatgccccggggctccttcgtgaggttggtatttctgatgctattgttaataactttttcctttccggcggccagattggtcaacaccgcccatcagttataccgcgcggcactgttgacagcaatgttgatacgctcgatgcgtttccaccttcctgtcaatttagcgcctaccatcagcttgcggaggaactcggtcatcgaccggctccgatcgctgccgttttgcccccctgcccagagctcgaacagggtctgctttatatgccccaggaactaactacgtccgacagcgtgcttacatttgaattaacagatatagtgcactgccgtatggcggctccaagccagcgcaaggcagtcctgtcgacccttgtcggcaggtatggccgtcgtacgaagttgtatgaggccgcccatgcggacgttcgcggatccctgaaccactttatccccgagctcggccccattagtgttaccacctgtgagctttacgagcttgtggaggctatggtagagaaggggcaggatggctctgcggtcttggagcttgatctatgtagccgtgatgtgtcgcgtataacattctttcagaaggattgtaataaatttacgacaggagagacaatagcacacggcaaagttgggcaggggatatctgcgtggagcaaaacgttttgtgccctatttggcccttggttccgtgccattgagaaggagattcttgcagcacttgctcccaatgttttctatggtgatgcatatgaagacacagtcctggccgctgctgttgctggggctcctggctgcaaggtttttgagaatgatttttctgagtttgatagcactcaaaataacttctcacttggtttggagtgtataatcatggaggagtgcggcatgccgcaatggatgatccgactttaccatcttgtccgctctgcctgggtcctgcaagccccaaaggagtccctgcggggtttctggaagaagcactcaggcgagcctggcaccttgctttggaacacggtctggaacatggcggtaatagcacactgttacgagttccgcgacttgaaagtcgcggcgtttaaaggggatgactctgttgtgctctgtagtgactaccggcagagccgcaatgcggccgccttaatcactggttgtgggctgaagcttaaggtggattttaggcccatcgggctgtatgctggtgttgttgtggccccgggtctcgggacccttcctgatgttgttaggttcgctgggcggctctcagagaaaaactgggggccgggtccagagagggctgagcagctacggctggcagtttgtgatttcctgcgaaagttaacgaatgtggctcaagtctgtgtggatgttgtttcgcgggtttatggtgttagccccggcttagtacataacctgattgggatgctccagactatcgccgatggcaaggcgcattttactgaaactgttaaacctgttttggatctcaccaattccattatacatcgggtggaatgaataacatgttcttttgctctgtgcatggagatgccaccatgcgctctcgggctcttctgtttctgctcttcgtgtttttgcctatgctgcccgcgccaccggccggtcagccgtctggccgtcgccgcgggcggcgcagcggcggtgccggcggtggtttctggggtgaccgggttgattctcagcccttcgccctcccctatattcatccaaccaaccccttcgcatctgacattccaaccgcagccggggctggagctcgccctcggcaaccggcccgtccactcggctccgcttggcgtgaccaatcccagcgccccaccgcttccgcccgtcgtcgacctgccccagctggggctgcgccgttgactgctgtggccccggctcctgataccgctcctgtccccgatgttgattctcgcggcgcaatattacgccgccaatataatttatctacatccccgcttacatctactattgctactggcactaaccttgttttatatgctgccccgttgagccctctgctcccgctccaagacggaactaatactcatattatggctactgaagcatcaaactacgcccagtatcgcgttgtccgcgccactattcggtaccgtccactcgtgccgaatgccgttggcgggtacgctatatctatttctttctggcctcagacgacaactactccgacatctgtcgatatgaattccatcacttccaccgatgttcggattcttgtccagcctggtattgcctctgaacttgtgatccctagcgagcgcctgcattaccgtaatcaaggctggcgttcggttgagacctctggtgtcgcagaggaggaggcaacctccggccttgttatgctttgcattcatggatcgcctgtgaattcttatactaatacaccctatactggtgcccttggtttgctcgattttgcactcgagcttgagttccgcaatttgacacctggtaatacaaatacgcgcgtttcccgatactcgagcagcgcgcgccacaagcttcgccgcgggcccgacggcactgctgaattgactaccactgctgctacacgctttatgaaagaccttcactttacagggactaatggtgttggtgaggttggtcgtggtatagcgctaactctgtttaatcttgctgatacgcttcttggcgggcttccgacagaattaatttcgtcggctggtggccagttgttttactcccgccccgtcgtctcagccaatggcgagccgactgtgaaactttacacctcagtcgagaatgctcagcaggacaagggtatagctattccacatgatattgatcttggcgagtcccgagttgttatccaggactatgacaaccaacatgagcaagaccgtcctactccctctccagctccctcccgtcctttttctgtccttcgtgctaatgatgtgctttggctttcacttacagctgctgagtatgatcagactacttatggctcttctactaaccctatgtatgtctctgataccgtgacatttgtcaatgtggctactggcgcccagggggtttcccgctccttggactggtctaaagttaccctcgatgggcgcccgcttactactattcagcagtactctaagaccttctttgtcctaccccttcgcggtaagctttccttctgggaggctggtactactaaggctggctatccgtacaattataataccactgctagtgaccagatcctaattgagaatgcggccggtcatcgtgtttgtatttcaacttatactactaatcttggctctggccctgtttctatctctgctgttggtgtcctcgcacctcattctgcgctggccgctctagaggacactgttgattatcccgctcgtgctcatacttttgacgatttctgccctgagtgccgtacgctcggccttcagggctgtgcttttcagtcaactgtcgctgagctacagcgtcttaaaatgaaggtgggtaaaacccgggagtattgatttattttgcttgtgccttccttctgttttgtttcttttatttctttcttctgcgcttcgcgctccctggaaaaaaaaaaaa

SEQ ID NO: 47 - DNA, 7228 bp, Hepatitis E virus Japan JKK-Sap (AB074917)
ccatggaggcccaccagttcataaaggctcctggcgtcactactgctattgagcaggcagctctagcagcggccaactccgccttggcgaatgctgtggtggttcggcctttcctatcccggctacagacagagatacttattaacctgatgcagccccggcagcttgttttccggcctgaagtcttgtggaaccacccaattcagcgcgtgatccacaacgagcttgagcagtactgccgggctcgggctggccgctgccttgaggtgggtgctcacccacgttctattaatgacaaccccaatgttctgcaccgctgtttcttgaaacctgtcggccgcgatgtccagcggtggtacaccgcccccactcgtggccccgcagcaaattgccgccggtcggccctccgtggcctcccacctgccgatcgaacctactgttttaatggtttttccggctgcacgtttgctgctgagacagggatagccctttactcactgcacgacctctggcctgccgatgttgccgaggcaatggctcgccatggtatgacccggctgtatgcagctctccacctccccccggaggtactgctcccccctggcacctatcataccacttcgtacctcctcatccatgatggggaccgtgcagtgattacatatgagggggactctagtgctggatacaaccatgatgtgtccattctgcgcgcctggatccgcactactaaggtcaccggcgaccacccgctggtaattgagcgggtccgggcggtgggctgtcactttgtgcttcttctcacagctgcacctgaaccatcgccaatgccctatgtcccataccctcgttccaccgaggtttatgttcggtccatttttggccctggtggttcaccatccctgtttccaactgcctgctctactaagtcaacatttcatgccgtccctgtgcatatatgggacaggcttatgctcttcggtgcgaccctcgatgaccaggccttttgctgttcaaggctaatgacataccttcgtggtattagttacaaagtcacagttggcgctcttgtcgccaacgagggctggaatgcctccgaagacgcattaactgctgtcattactgcggcttatcttaccatctgccatcagaggtacctccgcacacaggctatctcgaaagggatgaaaagattggagcttgagcatgcacagaagtttataacacgtctttatagctggttgtttgaaaaatctggccgtgattacatccccggccgtcagttgcagttttacgcccagtgccgtcgatggctatctgctggtttccaccttgatcctcgtgtgctcgtttttgatgaagcggccccctgccgctgtcgtagcttgcttcgcaaagctgcccataaattttgctgctttatgcgatggctagggcaggattgcacctgcttccttcagcctgttgaggggagggtcggtgaacagggttatgataacgaggcatttgaggggtcggatgtcgaccctgctgaagaggccactgtgagtatctctggctcgtacattgtcaccggcagccagttgcagcctctttaccaggcgctcggtattccttctgaccttgccgcccgagcgggccggctcactgctaccgtcgaagtttcagataccgacggccgtctcacttgtaagaccactatgggcaataagaccttcacaacagtttttaccgacggtgcccagctggaggctaatgggccggagcagtatgtgctatcatttgatttggctaaacagactatggccgccggcccgcatagccttagttacgccttgacacctgcgggtcttgaggtgcgtgttgtttctgccggactcgactgtagggctgttttcccatctggggttgcaaccccctctgcttccggggaggtgtccgccttctgttcggctttatacaggtttaaccgctgtgtccagcggcattctctcattggtggcttgtggtattatcctgaggggctaattggtttgtttccaccgttcgcccctggccacacctgggagtcagccaatcccttctgtggggagagcaccctttatactcgcacctggtcggtgtcgggcttttctagctgtttttctccattcgagccccccgctctggactcaccaccccccgccgaggctgatacacctatggctgtcgatgttccacccccagctaccttgacattaccacaacctccggctcctgaacgggcagtacccccgcaagatcttgccgatggcgatgtcgctcgggcctctccgggtgtctctgctgcaccccctgtgccggcgcaatccgtgactgatccacccgtgtcccgccggcggttacttcatacttatcctgatgggtcaaaagtgtacgctggctccctctttgagtctgagtgtacttggttggtcaatgcgtccaatcccggccaccgtcccggtggtggcctttgccatgcattttaccagcggttcccggagtcatttgaccccgccgagttcgtcatgtccgatgggtttgcagcttataccttgacgccccgacctattatccatgctgttgctcccgactatcgggtagaacataaccccaagaggctcgaggctgcttatcgagagacgtgctcccgccgtggcacggctgcttaccccctgcttggtgccggcatatataaggtgcctgttgggcttagttttgatgcctgggaacgcaaccatcggcccggggatgagttgtaccttactgagccggctatagcttggttcgaggctaaccgacccactctccccgcgctcactatcactgaggacacggcacgaacagctaatctggcgttggagctagattcggccactgaggtcggtcgggcgtgtgctggttgccgtgttgagccaggcgtcgttcactaccagttcacggcgggtgtccctggttccggcaagtcacggtcaattcagcagggcgaggtggatgtggtggtagtaccaactcgtgagctgcgtaactcatggcgacgccgcgggtttgcagcctacacaccccacactgcggcccgtgttactcgtggccgtagggttgtcattgatgaggccccgtcacttccaccacatctgctcttgctgcacatgcagcgggcctcgtcggtccaccttcttggcgaccccaaccaaatccctgccattgatttcgagcatgctggtcttgtacccgcaatccggcctgagcttgtaccaacaaagtggtggcatcttacccacaggtgcccggcagatgtttgtgagctaattcgcggcgcgtaccctaagattcagacagcgagccgtgtttttcgctccctgttttggggggagccccctgtcggccaaaaattagtgttcactcaggcggcgaaggccgctaaccctggtgcgattacggtccatgaggcccagggcgctacattcactgagactactattattgctacggcggatgctcgcgggctgattcagtcatctagggctcacgctatcgtggccctaacccgtcatacggagaaatgtgtggtcgttgatgccccgggactccttcgtgaggtcggtatctctgacgccattgttaataactttttcctttctggtggccagattggtcagcaccgtccatcagtcataccgcgtggcactatcaacaacaatgttgatacgcttgatgcattcccaccctcttgccagttcagcgcttaccaccagcttgcggaagagctcggccatcgaccggccccgatcgctgccgttctgcccccctgtcctgaacttgaacagggtctactttacatgcctcaggaattgaccacgtctgatagtgtgcttacgtttgaattgacagatatagtgcactgtcgtatggcggcacctagtcagcgtaaggcggtcctgtcgactcttgtcggcaggtacggccgccgtacgaagttgtatgaggctgcccatgcagatgtccgtgggtctctaaaccattttatccctgagctcggccctgttagtgttaccacttgtgaactttacgagcttgtggaggccatggtggagaaaggccaggatggttctgcggttctggagctcgatttatgtagccgtgatgtgtctcgtataacattcttccagaaggattgtaataagtttacaacgggcgagacaatagcacatggcaaagttggccaggggatatctgcatggagtaaaaccttttgtgctttgttcggcccatggttccgcgccattgagaaggagatccttgctgtacttgctcccaatgtattctatggtgatgcatacgaagatacagttctggccgccgcagtcgccggagcccctggttgcaaggttttcgagaatgatttctcagagtttgatagcactcaaaataatttttcacttgggctggagtgtataatcatggaggagtgcggcatgccgcagtggatgatccggctttatcaccttgttcgctctgcttgggttttgcaagctccaaaggagtctctgcgggggttctggaagaagcactcaggcgagcctggcaccttgctttggaatactgtctggaacatggcggtcatagcccattgttatgagttccgcgacttaaaagtcgccgcatttaaaggagatgactctgttgtgctttgtagtgattatcggcaaagccgcgatgcggccgccctaatcgctggctgtgggttgaaacttaaggtggattttaggcccattgggctgtatgctggtgttgttgtggccccgggtctcgggacccttcctgatgttgttaggtttgctgggcggctctcggagaaaaactgggggccgggttcagagagggcagaacagctacggctggctgtctgtgattttctgcgaaagttaacgaatgtggctcaagtctgtgtggacgttgtttcgcaggtttatggtgttagccccggtttggtacataacctgattgggatgctccagaccattgctgatggcaaggcgcactttacagagacagttaaacctgttttagacctcaccaattctattatacatcgggtggaatgaataacatgttcttttgctctgtgcatggagatgccaccatgcgctctcgggcttttctgtttttgttcctcgtgcttctgcctatgctgcccgcgccaccggccggtcagccgtctggccgtcgccgcgggcggcgcagcggcggtgccggcggtggtttctggggtgaccgggttgattctcagcccttcgccctcccctatattcatccaaccaaccccttcgcatctgacattccagccgcagccggggctggagctcgccctcggcagccagcccgtccactcggctccgcttggcgtgaccaatcccagcgccccgccacttccgcccgtcgtcgatctgccccagctggggcttcgccgctgactgctgtggccccggccccagatactgctcctgttcccgatgtcgattctcgcggcgctatattacgccgccagtataatttatcaacatccccgctaacatctactattgccactggtactaaccttgttctatatgctgccccgctgagccctttgcttccgctccaagatggaactaacactcacattatggccactgaagcatcaaattatgcccagtaccgtgttgtccgcgccaccatccggtaccgtccgcttgtgccgaacgctgtcggcggatacgctatatctatctctttctggcctcagacaactaccaccccgacatctgtggatatgaattctatcacctccacggatgtccgaatccttgtccagcctggcatcgcttcagaacttgtgatccctagtgagcgcctgcattatcgtaaccaaggctggcgctcggttgagacctctggtgttgcggaggaggaggcgacctccggccttgtcatgctttgcatccatggatcacctgtgaattcttacaccaatacgccttatactggtgcccttggcttgcttgatttcgcactcgagctcgagttccgcaatttgacacccggtaacacgaacacacgtgtttcccgctactcgagtagtgcgcgccacaagctacgccgagggcctgatggcactgctgagttaactaccactgctgctacacgctttatgaaggaccttcattttacagggactaatggagttggtgaagtcggtcgtggtatagcgctaactctgttcaatcttgctgacacgcttctcggcgggctcccgacagaattgatttcgtcggctggtggtcagttgttttactctcgccccgtcgtctcagccaatggcgagccgacagtgaagctttatacttcagtcgagaacgctcagcaggataagggtatagctatcccacatgatattgaccttggtgagtcccgtgttgtcattcaggattacgataaccaacatgagcaggaccgtcccaccccttctcctgctccctctcggcccttttctgtccttcgtgctaatgatgtgctatggctttcacttacagcagctgagtatgatcagactacctatggctcctctactaaccccatgtatgtctctgatactgtgacatttgttaatgtggctactggtgcccagggggtatctcgctctctggactggtctaaagttacccttgatgggcgcccacttatgactatccagcagtattctaagaccttctttgtcctgcctctccgtggtaagctctccttttgggaggctggtactactaaggccggctacccttataattataatactaccgccagtgaccagattttaattgagaatgcagctggtcaccgtgtatgcatttcaacctacactactaatcttggatctggccctgtttctatttctgccgtcggtgtcctcgcacctcactctgcgctggccgctttagaggacactgttgactatcctgcccgtgctcatacttttgatgatttttgccctgagtgccgtgcactcggccttcagggttgtgctttccaatcgactgttgctgagctacagcgccttaaaatgaaggtgggtaaaacccgggagtactgatttattctacttgtaccttccttctgttctgttcttttattttycttttctgcgtyccgcgctccctggaaaaaaaaaaaa

SEQ ID NO: 48 - DNA, 7230 bp, Hepatitis E virus Japan JRA1 (AP003430)
ggcagaccacgtatgtggtcgatgccatggaggcccaccagttcattaaggctcctggcattactactgccatagagcaggctgctctggctgcggccaactccgccttggcgaatgctgtggtggttcggccgtttttgtctcgcgtacaaaccgagatccttattaatttgatgcaaccccggcagttggttttccgccctgaggtgctttggaaccatcctattcagagggttatacacaatgaattagaacagtactgccgggcccgggccggtcgttgcctggagattggggctcatccaagatctattaatgataaccccaatgttttgcaccggtgttttctcaggccggtcggtagggacgtccagcgctggtattctgcccccacccgcggccctgcagccaactgccgccgctctgcattgcgtggcctcccccctgtcgaccgcacttactgttttgatggattttccagttgtgctttcgccgcagagaccggcgtggccctttactctctacatgacctttggccagctgatgttgcggaggctatggcccgccatggaatgacacgtctttatgctgcactccacctccctcccgaggtgttgttaccacccggtacttaccacacaacctcgtatcttctgatccatgacggcaaccgtgctgttgtgacttatgaaggtgatactagtgcaggttacaaccatgacgtttccatacttcgtgcatggattcgcacgactaaaatagttggtgaccatccgttggttatagagcgtgtgcgggctattggttgtcattttgtgctgctgctcaccgcggcccctgagccatcgcccatgccttacgtcccttacccccgttccacggaggtgtatgtccgatctatatttggccctggtggctccccatccttgtttccgtcagcttgctctacaaaatctaccttccatgctgttccagttcacatatgggaccggcttatgctttttggtgccaccttggacgaccaggcgttttgctgctcacggctcatgacatacctccgtggcattagctacaaagtcactgtcggtgcgcttgttgctaatgaggggtggaatgcctctgaagatgccctcaccgcagtaatcactgcagcctacttgaccatctgtcaccagcgctacctccgtacccaggcgatatctaagggtatgcgccggttggaggttgagcacgcccagaagtttattacaagactttacagctggttatttgagaagtctggtcgtgactacatccccggccgccaacttcagttctatgcccagtgccggcggtggctatctgcgggctttcacctagaccccagagtgctcgtctttgatgagtcagtgccctgccgctgtaggactttcttaaagaaggttgcgggtaaattctgttgcttcatgcggtggttagggcaggagtgtacttgtttcttagaaccagctgagggcttagttggcgatcaaggccatgataatgaggcttatgagggttctgaggttgaccaggccgagcctgtacaccttgatgtgtcggggacctatgctgtccacgggcaccagcttgaggccctctacagggcactcaacattccacacgatattgctgctcgagctgcccgattaacggctactgttgagctcgctgcaggccccgaccgtctggagtgccgcaccgtgctcgggaacaagactttccggacgacggtgactgatggcgcccatctagaagcgaacggccctgagcaatatgttctgtcgttcgatgcctcccgccagtctatgggggccgggtcccatagcctcacttacgagcttacacccgccggcttgcaggttaagatctcatctaatggcctagactgcactgccgtattcccccctggtggcgcccctagcgcgccgccgggggaggtggcggctttttgcagcgccctctacaggtataacagattcacccagcggcattcgctgaccggcggtttgtggctacatcccgaggggttgctaggcgttttcccccctttttcccctgggcacatttgggagtctgccaaccccttctgcggtgagggtaccttgtatactcgcacctggtctacatctggtttttctagtgacttttcccctcctgaggcggccgcccctgcaccggctgccgccccagggtcgtcctcccccactccaccagtcagtgatatttgggtgttaccaccgctttcagaggagccccaggtgggtgcgccgcctgcacctcccacccccgagcctgctcggctgccctgccccactaaacctaacacccccgtgcgtaagccaacggcaccgccgccttctcgcacccgccgccttctttacacctatcctgatggtgctaaggtgtatgcggggtcactgtttgagtcagattgtgattggctggtgaatgcctctaacccgggccatcgtcctgggggtggcctctgtcacgccttttaccaacgttttcccgaggcgttctatccaactgaatttattatgcgtgagggtcttgcagcatacaccttgaccccgcgccctattatccatgcagtggcccccgactatagggttgagcagaatccaaagaggcttgaggcagcgtaccgagaaacttgctcccgccgcggcaccgctgcctatccacttcttggctcaggtatataccaggtccctgctggtcttagttttgatgcctgggagcgtaaccatcgccctggcgatgagctttacctgactgaacccgctgcagcctggtttgaagctaataagccaacgcagccagcacttacgataacggaagatacagctcgcacggccaacctagcattagagatcgatgctgccacagacgttggccgtgcttgtgccggctgcactattagccccgggattgtgcattatcagttcactgccggggtcccaggctcaggcaaatcaaggtccatacagcagggggatgtcgatgttgtggttgtccccacccgggaacttcgcaatagctggcgccgtcggggctttgcggcctttacaccccacacggcggcccgtgttactataggccgccgtgttgttattgacgaggccccatcccttccaccgcacttgttgctgttacacatgcagcgggcctcctcagtccatctcctcggtgacccaaatcagatccctgccatcgacttcgagcatgccggcctggtccccgcaattcgccctgagcttgcaccaacgagctggtggcatgttacgcatcgctgtccggccgatgtatgtgagctcatacgcggagcctaccctaaaatccagaccacgagccgtgtgctacggtctctgttctggaatgagccggctattggccagaagttggtcttcacgcaggccgctaaagctgccaaccctggtgcaattacggtccatgaagcccagggtgctaccttcacagagaccacaattatagccacggctgatgccaggggtcttattcagtcatcccgggcccatgctatagttgcacttacccgccacacagagaagtgcgttattttggatgcccctggtttgttgcgcgaggtcggcatatcagatgtaattgttaacaactttttccttgctggcggagaggtgggccatcaccgcccctctgtgatacctcgtggcaatcctgaccggaaccttgacaccctacaggccttcccgccatcctgccaaattagtgcttaccatcagttggctgaggagttaggccatcgcccggctcctgttgctgccgttttgcccccctgccccgagctcgagcagggcttattgtatatgccacaggagcttacagtgtccgatagtgtgttggtttttgagctcactgacatagtccattgccgcatggctgccccaagtcagcggaaggccgtcctctcaacacttgtggggaggtatggccgcaagacgaaattgtatgaggcagcccattcagatgttcgagagtctctagctaggttcattcccactatcggacctgtccaggccaccacgtgtgagttatatgaactggttgaggccatggtggaaaagggccaggacggctctgccgtcctggagcttgatctatgtaatcgtgatgtttcgcgcattacattctttcagaaagactgtaataagtttacaactggtgagaccattgcccatggtaaggttggccagggcatatcggcctggagtaagaccttttgcgccctgtttggtccgtggtttcgtgccattgaaaaagaaatactagccctgctcccgcctaatatcttctacggcgacgcctacgaggagtcggtgtttgccgcggccgtgtccggggcggggtcttgcatggtatttgaaaatgacttttcggaatttgatagtactcagaacaatttctcccttggccttgagtgtgtggttatggaagagtgtggtatgccccaatggctgatcagattgtatcacctggtccggtcagcctggattctgcaggcgccaaaggagtctcttaaaggtttctggaagaagcattctggtgagcctggtacccttctctggaacaccatctggaacatggcgatcatagcccattgctatgagtttcgcgatttccgggtcgccgcttttaagggtgatgactcggtagtcctctgtagtgattaccgacagagtcgtaacgcggcagccttaatcgcaggttgtgggctcaagttgaaggttgactatcgccctattgggctgtacgctggtgtggtggtggcccctggcttggggacactgcctgatgtagtgcgatttgctggccggctgtccgaaaagaattggggccctggcccagagcgtgctgagcagctgcgtcttgctgtctgtgacttccttcgagggttaacgaatgttgcgcaggtttgtgttgatgttgtgtcccgtgtttatggagtcagccccgggctggtacataaccttattggcatgctgcagaccattgctgatggcaaggcccactttacagagtcaattaaacctgtgcttgaccttacaaattctattatacagcgggtggaatgaataacatgtcttgtgcatcgcccatgggatcaccatgcgccctagggctgttctgttgttgctcttcgtgcttctgcctatgctgcccgcgccaccggccggccagccgtctggccgccgtcgtgggcggcgcagcggcggtgccggcggtggtttctggggtgacagggttgattctcagcccttcgccctcccctatattcatccaaccaaccccttcgccgccgatgtcgtttcacaatccggggctggagctcgccctcgacagccgccccgcccccttggctccgcttggcgcgaccagtcccagcgccccaccgctgccccccgtcgtcgacctgccccagctggggctgcgccgctgactgctacatcaccagctcctgacacagctcctgtgcctgatgtcgactcacgcggcgctattttgcgtcggcagtataatctgtccacgtccccgcttacgtcatctgtcgcctcgggtactaatttggttctctatgccgccccattgaacccactcctacccctccaggatggcaccaacacccacatcatggctaccgaggcatctaattatgcccagtaccgggtggttcgagctacaatccgttatcgcccgttggtaccaaatgctgttggtggttatgccatctctatttccttttggcctcagacaaccactacccccacctctgttgatatgaattctattacctctactgatgttaggatcttagtccagcccggtattgcctccgagttggtcattcccagtgagcgtcttcattatcgtaatcaaggctggcgctctgtcgagaccacgggtgtcgccgaggaggaggctacctctggcctggtaatgctttgcattcatgggtctcctgtcaattcttacacaaatacaccttatactggagcactggggcttcttgattttgcattagagcttgagtttaggaatttgacacctgggaacactaatacccgtgtgtcccgatataccagcacagcccgccaccggctgcgccgtggtgccgacgggaccgctgagcttaccactacggcggccacgcgttttatgaaggacctgcatttcaccggtacgaatggagttggtgaggtgggccgaggcatagccctgacactgttcaatcttgctgatacgcttctcggtggtctgccgacagaattgatttcgtcggctgggggccagttgttctactcccgccctgtcgtctcagccaatggcgaaccgactgtaaagttatatacatctgttgagaatgcacagcaggataagggtatcaccatcccacacgatatagacctcggtgattctcgtgtggtcatccaggactatgataaccagcacgagcaggaccgacctaccccctcacccgccccctctcgtccattctcggttcttcgcgctaatgatgttctgtggctctctcttaccgctgccgagtatgaccagactacgtatgggtcatctaccaaccccatgtatgtctcggacacagtcacatttgttaacgtggccactggtgctcaggctgttgctcgctcccttgattggtctaaagttaccttggacggtcgccccctcactactattcagcagtattctaagacattctacgttctcccgcttcgtggaaagttgtctttttgggaggctggcacgaccaaggctggttatccttataattataatacaactgctagtgatcagattttaatagagaacgcagctggccatcgtgtcgctatctctacttacactaccagcctgggcgccggccccacttctatctctgcggtcggtgtgttagccccgcactcagcccttgctgttcttgaggataccgccgattaccccgctcgtgcccatacttttgatgacttctgcccggaatgtcgcacccttggtttgcagggctgcgctttccagtctactattgctgagcttcagcgccttaaaatgaaggtaggtaaaacccgggagttctaattaattcgtcctgtacccccttcgtagttttcttttgctttatttctcttttctgctttccgcgctccctggaaa

SEQ ID NO: 49 - DNA, 7180 bp, Hepatitis E virus Mexico (M74506)
gccatggaggcccaccagttcattaaggctcctggcatcactactgctattgagcaagcagctctagcagcggccaactccgcccttgcgaatgctgtggtggtccggcctttcctttcccatcagcaggttgagatccttataaatctcatgcaacctcggcagctggtgtttcgtcctgaggttttttggaatcacccgattcaacgtgttatacataatgagcttgagcagtattgccgtgctcgctcgggtcgctgccttgagattggagcccacccacgctccattaatgataatcctaatgtcctccatcgctgctttctccaccccgtcggccgggatgttcagcgctggtacacagccccgactaggggacctgcggcgaactgtcgccgctcggcacttcgtggtctgccaccagccgaccgcacttactgttttgatggctttgccggctgccgttttgccgccgagactggtgtggctctctattctctccatgacttgcagccggctgatgttgccgaggcgatggctcgccacggcatgacccgcctttatgcagctttccacttgcctccagaggtgctcctgcctcctggcacctaccggacatcatcctacttgctgatccacgatggtaagcgcgcggttgtcacttatgagggtgacactagcgccggttacaatcatgatgttgccaccctccgcacatggatcaggacaactaaggttgtgggtgaacaccctttggtgatcgagcgggtgcggggtattggctgtcactttgtgttgttgatcactgcggcccctgagccctccccgatgccctacgttccttacccgcgttcgacggaggtctatgtccggtctatctttgggcccggcgggtccccgtcgctgttcccgaccgcttgtgctgtcaagtccacttttcacgccgtccccacgcacatctgggaccgtctcatgctctttggggccaccctcgacgaccaggccttttgctgctccaggcttatgacgtaccttcgtggcattagctataaggtaactgtgggtgccctggtcgctaatgaaggctggaatgccaccgaggatgcgctcactgcagttattacggcggcttacctcacaatatgtcatcagcgttatttgcggacccaggcgatttctaagggcatgcgccggcttgagcttgaacatgctcagaaatttatttcacgcctctacagctggctatttgagaagtcaggtcgtgattacatcccaggccgccagctgcagttctacgctcagtgccgccgctggttatctgccgggttccatctcgacccccgcaccttagtttttgatgagtcagtgccttgtagctgccgaaccaccatccggcggatcgctggaaaattttgctgttttatgaagtggctcggtcaggagtgttcttgtttcctccagcccgccgaggggctggcgggcgaccaaggtcatgacaatgaggcctatgaaggctctgatgttgatactgctgagcctgccaccctagacattacaggctcatacatcgtggatggtcggtctctgcaaactgtctatcaagctctcgacctgccagctgacctggtagctcgcgcagcccgactgtctgctacagttactgttactgaaacctctggccgtctggattgccaaacaatgatcggcaataagacttttctcactacctttgttgatggggcacgccttgaggttaacgggcctgagcagcttaacctctcttttgacagccagcagtgtagtatggcagccggcccgttttgcctcacctatgctgccgtagatggcgggctggaagttcatttttccaccgctggcctcgagagccgtgttgttttcccccctggtaatgccccgactgccccgccgagtgaggtcaccgccttctgctcagctctttataggcacaaccggcagagccagcgccagtcggttattggtagtttgtggctgcaccctgaaggtttgctcggcctgttcccgcccttttcacccgggcatgagtggcggtctgctaacccattttgcggcgagagcacgctctacacccgcacttggtccacaattacagacacacccttaactgtcgggctaatttccggtcatttggatgctgctccccactcgggggggccacctgctactgccacaggccctgctgtaggctcgtctgactctccagaccctgacccgctacctgatgttacagatggctcacgcccctctggggcccgtccggctggccccaacccgaatggcgttccgcagcgccgcttactacacacctaccctgacggcgctaagatctatgtcggctccattttcgagtctgagtgcacctggcttgtcaacgcatctaacgccggccaccgccctggtggcgggctttgtcatgctttttttcagcgttaccctgattcgtttgacgccaccaagtttgtgatgcgtgatggtcttgccgcgtatacccttacaccccggccgatcattcatgcggtggccccggactatcgattggaacataaccccaagaggctcgaggctgcctaccgcgagacttgcgcccgccgaggcactgctgcctatccactcttaggcgctggcatttaccaggtgcctgttagtttgagttttgatgcctgggagcggaaccaccgcccgtttgacgagctttacctaacagagctggcggctcggtggtttgaatccaaccgccccggtcagcccacgttgaacataactgaggataccgcccgtgcggccaacctggccctggagcttgactccgggagtgaagtaggccgcgcatgtgccgggtgtaaagtcgagcctggcgttgtgcggtatcagtttacagccggtgtccccggctctggcaagtcaaagtccgtgcaacaggcggatgtggatgttgttgttgtgcccactcgcgagcttcggaacgcttggcggcgccggggctttgcggcattcactccgcacactgcggcccgtgtcactagcggccgtagggttgtcattgatgaggccccttcgctccccccacacttgctgcttttacatatgcagcgtgctgcatctgtgcacctccttggggacccgaatcagatccccgccatagattttgagcacaccggtctgattccagcaatacggccggagttggtcccgacttcatggtggcatgtcacccaccgttgccctgcagatgtctgtgagttagtccgtggtgcttaccctaaaatccagactacaagtaaggtgctccgttcccttttctggggagagccagctgtcggccagaagctagtgttcacacaggctgctaaggccgcgcaccccggatctataacggtccatgaggcccagggtgccacttttaccactacaactataattgcaactgcagatgcccgtggcctcatacagtcctcccgggctcacgctatagttgctctcactaggcatactgaaaaatgtgttatacttgactctcccggcctgttgcgtgaggtgggtatctcagatgccattgttaataatttcttcctttcgggtggcgaggttggtcaccagagaccatcggtcattccgcgaggcaaccctgaccgcaatgttgacgtgcttgcggcgtttccaccttcatgccaaataagcgccttccatcagcttgctgaggagctgggccaccggccggcgccggtggcggctgtgctacctccctgccctgagcttgagcagggccttctctatctgccacaggagctagcctcctgtgacagtgttgtgacatttgagctaactgacattgtgcactgccgcatggcggcccctagccaaaggaaagctgttttgtccacgctggtaggccggtatggcagacgcacaaggctttatgatgcgggtcacaccgatgtccgcgcctcccttgcgcgctttattcccactctcgggcgggttactgccaccacctgtgaactctttgagcttgtagaggcgatggtggagaagggccaagacggttcagccgtcctcgagttggatttgtgcagccgagatgtctcccgcataacctttttccagaaggattgtaacaagttcacgaccggcgagacaattgcgcatggcaaagtcggtcagggtatcttccgctggagtaagacgttttgtgccctgtttggcccctggttccgtgcgattgagaaggctattctatcccttttaccacaagctgtgttctacggggatgcttatgacgactcagtattctctgctgccgtggctggcgccagccatgccatggtgtttgaaaatgatttttctgagtttgactcgactcagaataacttttccctaggtcttgagtgcgccattatggaagagtgtggtatgccccagtggcttgtcaggttgtaccatgccgtccggtcggcgtggatcctgcaggccccaaaagagtctttgagagggttctggaagaagcattctggtgagccgggcagcttgctctggaatacggtgtggaacatggcaatcattgcccattgctatgagttccgggacctccaggttgccgccttcaagggcgacgactcggtcgtcctctgtagtgaataccgccagagcccaggcgccggttcgcttatagcaggctgtggtttgaagttgaaggctgacttccggccgattgggctgtatgccggggttgtcgtcgccccggggctcggggccctacccgatgtcgttcgattcgccggacggctttcggagaagaactgggggcctgatccggagcgggcagagcagctccgcctcgccgtgcaggatttcctccgtaggttaacgaatgtggcccagatttgtgttgaggtggtgtctagagtttacggggtttccccgggtctggttcataacctgataggcatgctccagactattggtgatggtaaggcgcattttacagagtctgttaagcctatacttgaccttacacactcaattatgcaccggtctgaatgaataacatgtggtttgctgcgcccatgggttcgccaccatgcgccctaggcctcttttgctgttgttcctcttgtttctgcctatgttgcccgcgccaccgaccggtcagccgtctggccgccgtcgtgggcggcgcagcggcggtaccggcggtggtttctggggtgaccgggttgattctcagcccttcgcaatcccctatattcatccaaccaacccctttgccccagacgttgccgctgcgtccgggtctggacctcgccttcgccaaccagcccggccacttggctccacttggcgagatcaggcccagcgcccctccgctgcctcccgtcgccgacctgccacagccggggctgcggcgctgacggctgtggcgcctgcccatgacacctcacccgtcccggacgttgattctcgcggtgcaattctacgccgccagtataatttgtctacttcacccctgacatcctctgtggcctctggcactaatttagtcctgtatgcagccccccttaatccgcctctgccgctgcaggacggtactaatactcacattatggccacagaggcctccaattatgcacagtaccgggttgcccgcgctactatccgttaccggcccctagtgcctaatgcagttggaggctatgctatatccatttctttctggcctcaaacaaccacaacccctacatctgttgacatgaattccattacttccactgatgtcaggattcttgttcaacctggcatagcatctgaattggtcatcccaagcgagcgccttcactaccgcaatcaaggttggcgctcggttgagacatctggtgttgctgaggaggaagccacctccggtcttgtcatgttatgcatacatggctctccagttaactcctataccaataccccttataccggtgcccttggcttactggactttgccttagagcttgagtttcgcaatctcaccacctgtaacaccaatacacgtgtgtcccgttactccagcactgctcgtcactccgcccgaggggccgacgggactgcggagctgaccacaactgcagccaccaggttcatgaaagatctccactttaccggccttaatggggtaggtgaagtcggccgcgggatagctctaacattacttaaccttgctgacacgctcctcggcgggctcccgacagaattaatttcgtcggctggcgggcaactgttttattcccgcccggttgtctcagccaatggcgagccaaccgtgaagctctatacatcagtggagaatgctcagcaggataagggtgttgctatcccccacgatatcgatcttggtgattcgcgtgtggtcattcaggattatgacaaccagcatgagcaggatcggcccaccccgtcgcctgcgccatctcggcctttttctgttctccgagcaaatgatgtactttggctgtccctcactgcagccgagtatgaccagtccacttacgggtcgtcaactggcccggtttatatctcggacagcgtgactttggtgaatgttgcgactggcgcgcaggccgtagcccgatcgcttgactggtccaaagtcaccctcgacgggcggcccctcccgactgttgagcaatattccaagacattctttgtgctcccccttcgtggcaagctctccttttgggaggccggcacaacaaaagcaggttatccttataattataatactactgctagtgaccagattctgattgaaaatgctgccggccatcgggtcgccatttcaacctataccaccaggcttggggccggtccggtcgccatttctgcggccgcggttttggctccacgctccgccctggctctgctggaggatacttttgattatccggggcgggcgcacacatttgatgacttctgccctgaatgccgcgctttaggcctccagggttgtgctttccagtcaactgtcgctgagctccagcgccttaaagttaaggtgggtaaaactcgggagttgtagtttatttggctgtgcccacctacttatatctgctgatttcctttatttcctttttctcggtcccgcgctccctgaaaaaaaaaa

SEQ ID NO: 50 - protein, 1709 aa, Hepatitis E virus JKN-Sap (ORF1)
MEAHQFIKAPGITTAIEQAALAAANSALANAVVVRPFLSRVQTEILINLMQPRQLVFRPEVLWNHPIQRVIHNELEQYCRARAGRCLEVGAHPRSINDNPNVLHRCFLRPVGRDVQRWYSAPTRGPAANCRRSALRGLPPADRTYCFDGFSRCAFAAETGVALYSLHDLWPADVAEAMARHGMTRLYAALHLPPEVLLPPGTYHTTSYLLIHDGDRAVVTYEGDTSAGYNHDVSILRAWIRTTKIVGDHPLVIERVRAIGCHFVLLLTAAPEPSPMPYVPYPRSTEVYVRSIFGPGGSPSLFPSACSTKSTFHAVPVHIWDRLMLFGATLDDQAFCCSRLMTYLRGISYKVTVGALVANEGWNASEDALTAVITAAYLTICHQRYLRTQAISKGMRRLEVEHAQKFITRLYSWLFEKSGRDYIPGRQLQFYAQCRRWLSAGFHLDPRVLVFDEAVPCRCRTFLKKVAGKFCCFMRWLGQECTCFLEPAEGLIGDQGHDNEAYEGSEVDPAEPAHLDVSGTYAVHGHQLEALYRALNVPHDIAARASRLTATVELVASPDRLECRTVLGNKTFRTTVVDGAHLEANGPEEYVLSFDASRQSMGAGSHSLTYELTPAGLQVRISSNGLDCTAVFPPGGAPSAAPGEVAAFCSALYRYNRFTQRHSLTGGLWLHPEGLLGIFPPFSPGHIWESANPFCGEGTLYTRTWSTSGFSSDFSPPEAAAPVPAAAPGLPHPTPPVSDIWVLPPPSEESQIDAAPVPPVPKTVGLPSPIVLAPPSPLPSPVRKPPSPPPSRTRRLLYTYPDGARVYAGSLFESDCDWLVNASNPGHRPGGGLCHAFYQRFPEAFYPTEFIMREGLAAYTLTPRPIIHAVAPDYRVEQNPKRLEAAYRETCSRRGTAAYPLLGSGIYQVPVSLSFDAWERNHRPGDELYLTEPAAAWFEANKPSQPALTITEDTARTANLALEIDAATEVGRACAGCTISPGVVHYQFTAGVPGSGKSRSIQQGDVDVVVVPTRELRNSWRRRGFAAFTPHTAARVTIGRRVVIDEAPSLPPHLLLLHMQRASSVHLLGDPNQIPAIDFEHAGLVPAIRPELAPTSWWHVTHRCPADVCELIRGAYPKIQTTSRVLRSLFWNEPAIGQKLVFTQAAKAANPGAITVHEAQGATFTETTIIATADARGLIQSSRAHAIVALTRHTEKCVILDAPGLLREVGISDVIVNNFFLAGGEVGHHRPSVIPRGNPDQNLGTLQAFPPSCQISAYHQLAEELGHRPAPVAAVLPPCPELEQGLLYMPQELTVSDSVLVFELTDIVHCRMAAPSQRKAVLSTLVGRYGRRTKLYEAAHSDVRESLARFIPTIGPVQATTCELYELVEAMVEKGQDGSAVLELDLCNRDVSRITFFQKDCNKFTTGETIAHGKVGQGISAWSKTFCALFGPWFRAIEKEILALLPPNIFYGDAYEESVFAAAVSGAGSCMVFENDFSEFDSTQNNFSLGLECVVMEECGMPQWLIRLYHLVRSAWILQAPKESLKGFWKKHSGEPGTLLWNTVWNMAIIAHCYEFRDFRVAAFKGDDSVVLCSDYRQSRNAAALIAGCGLKLKVDYRPIGLYAGVVVAPGLGTLPDVVRFAGRLSEKNWGPGPERAEQLRLAVCDFLRGLTNVAQVCVDVVSRVYGVSPGLVHNLIGMLQTIADGKAHFTETIKPVLDLTNSIIQREE

SEQ ID NO: 51 - protein, 660 aa, Hepatitis E virus JKN-Sap (ORF2)
MRPRAVLLLFLVLLPMLPAPPAGQPSGRRRGRRSGGAGGGFWGDRVDSQPFALPYIHPTNPFAADVVSQPGAGTRPRQPPRPLGSAWRDQSQRPSAAPRRRSAPAGAAPLTAVSPAPDTAPVPDVDSRGAILRRQYNLSTSPLTSSVASGTNLVLYAAPLNPLLPLQDGTNTHIMATEASNYAQYRVVRATIRYRPLVPNAVGGYAISISFWPQTTTTPTSVDMNSITSTDVRILVQPGIASELVIPSERLHYRNQGWRSVETTGVAEEEATSGLVMLCIHGSPVNSYTNTPYTGALGLLDFALELEFRNLTPGNTNTRVSRYTSTARHRLRRGADGTAELTTTAATRFMKDLHFTGTNGVGEVGRGIALTLFNLADTLLGGLPTELISSAGGQLFYSRPVVSANGEPTVKLYTSVENAQQDKGITIPHDIDLGDSRVVIQDYDNQHEQDRPTPSPAPSRPFSVLRANDVLWLSLTAAEYDQTTYGSSTNPMYVSDTVTLVNVATGAQAVARSLDWSKVTLDGRPLTTIQQYSKTFYVLPLRGKLSFWEAGTTKAGYPYNYNTTASDQILIENAAGHRVAISTYTTSLGAGPTSISAVGVLAPHSALAVLEDTVDYPARAHTFDDFCPECRTLGLQGCAFQSTIAELQRLKMKVGKTRES

SEQ ID NO: 52 - protein, 1709 aa, Hepatitis E virus JMY-Haw (ORF-1)
MEAHQFIKAPGITTAIEQAALAAANSALANAVVVRPFLSRVQTEILINLMQPRQLVFRPEVLWNHPIQRVIHNELEQYCRARAGRCLEVGAHPRSINDNPNVLHRCFLRPVGRDVQRWYSAPTRGPAANCRRSALRGLPPADRTYCFDGFSRCAFAAETGVALYSLHDLWPADVAEAMARHGMTRLYAVLHLPPEVLLPPGTYHTTSYLLIHDGDRAVVTYEGDTSAGYNHDVSILRAWIRTTKIVGDHPLVIERVRAIGCHFVLLLTAAPEPSPMPYVPYPRSTEVYVRSIFGPGGSPSLFPSACSTKSTFHAVPVHIWDRLMLFGATLDDQAFCCSRLMTYLRGISYKVTVGALVANEGWNASEDALTAVITAAYLTICHQRYLRTQAISKGMRRLEVEHAQKFITRLYSWLFEKSGRDYIPGRQLQFYAQCRRWLSAGFHLDPRVLVFDESVPCRCRTFLKKVAGKFCCFMRWLGQECTCFLEPAEGLVGDQGHDNEAYEGSEVDPAEPAHLDVSGTYAVHGHQLEALYRALNVPHDIAARASRLTATVELVASPDRLECRTVLGNKTFRTTVVDGAHLEANGPEQYVLSFDASRQSMGAGSHSLTYELTPAGLQVRISSNGLDCTAVFPPGGAPSAAPGEVAAFCSALYRYNRFTQRHSLTGGLWLHPEGLLGIFPPFSPGHIWESANPFCGEGTLYTRTWSTSGFSSDFSPPEAAAPVPAAAPGLPHPTPPVSDIWVLPPPSEGSQIDAAPVPPVPKTVGLPSPIVLAPPPPFPSPVRKPSSPPPSRTRRLLYTYPDGARVYAGSLFESDCDWLVNASNPGHRPGGGLCHAFYQRFPEAFYPTEFIMREGLAAYTLTPRPIIHAVAPDYRVEQNPKRLEAAYRETCSRRGTAAYPLLGSGIYQVPVSLSFDAWERNHRPGDELYLTEPAAAWFEANKPSQPALTITEDTARTANLALEIDAATEVGRACAGCTISPGIVHYQFTAGVPGSGKSRSIQQGDVDVVVVPTRELRNSWRRRGFAAFTPHTAARVTIGRRVVIDEAPSLPPHLLLLHMQRASSVHLLGDPNQIPAIDFEHAGLVPAIRPELAPTSWWHVTHRCPADVCELIRGAYPKIQTTSRVLRSLFWNEPAIGQKLVFTQAAKAANPGAITVHEAQGATFTETTIIATADARGLIQSSRAHAIVALTRHTEKCVILDAPGLLREVGISDVIVNNFFLAGGEVGHHRPSVIPRGNPDQNLGTLQAFPPSCQISAYHQLAEELGHRPAPVAAVLPPCPELEQGLLYMPQELTVSDSVLVFELTDIVHCRMAAPSQRKAVLSTLVGRYGRRTKLYEAAHSDVRESLARFIPTIGPVQATTCELYELVEAMVEKGQDGSAVLELDLCNRDVSRITFFQKDCNKFTTGETIAHGKVGQGISAWSKTFCALFGPWFRAIEKEILALLPPNIFYGDAYEESVFAAAVSGAGSCMVFENDFSEFDSTQNNFSLGLECVVMEECGMPQWLIRLYHLVRSAWILQAPKESLKGFWKKHSGEPGTLLWNTVWNMAIIAHCYEFRDFRVAAFKGDDSVVLCSDYRQSRNAAALIAGCGLKLKVDYRPIGLYAGVVVAPGLGTLPDVVRFAGRLSEKNWGPGPERAEQLRLAVCDFLRGLTNVAQVCVDVVSRVYGVSPGLVHNLIGMLQTIADGKAHFTETIKPVLDLTNSIIQREE

SEQ ID NO: 53 - protein, 660 aa, Hepatitis E virus JMY-Haw (ORF2)
MRPRAVLLLFLVLLPMLPAPPAGQPSGRRRGRRSGGTGGGFWGDRVDSQPFALPYIHPTNPFAADVVSQPGAGARPRQPPRPLGSAWRDQSQRPSAAPRRRSAPAGAAPLTAVSPAPDTAPVPDVDSRGAILRRQYNLSTSPLTSSVASGTNLVLYAAPLNPLLPLQDGTNTHIMATEASNYAQYRVVRATIRYRPLVPNAVGGYAISISFWPQTTTTPTSVDMNSITSTDVRILVQPGIASELVIPSERLHYRNQGWRSVETTGVAEEEATSGLVMLCIHGSPVNSYTNTPYTGALGLLDFALELEFRNLTPGNTNTRVSRYTSTARHRLRRGADGTAELTTTAATRFMKDLHFTGTNGVGEVGRGIALTLFNLADTLLGGLPTELISSAGGQLFYSRPVVSANGEPTVKLYTSVENAQQDKGITIPHDIDLGDSRVVIQDYDNQHEQDRPTPSPAPSRPFSVLRANDVLWLSLTAAEYDQTTYGSSTNPMYVSDTVTLVNVATGAQAVARSLDWSKVTLDGRPLTTIQQYSKTFYVLPLRGKLSFWEAGTTKAGYPYNYNTTASDQILIENAAGHRVAISTYTTSLGAGPTSISAVGVLAPHSALAALEDTVDYPARAHTFDDFCPECRTLGLQGCAFQSTIAELQRLKMKVGKTRES

SEQ ID NO: 54 - protein, 1707 aa, Hepatitis E virus JKK-Sap (ORF-1)
MEAHQFIKAPGVTTAIEQAALAAANSALANAVVVRPFLSRLQTEILINLMQPRQLVFRPEVLWNHPIQRVIHNELEQYCRARAGRCLEVGAHPRSINDNPNVLHRCFLKPVGRDVQRWYTAPTRGPAANCRRSALRGLPPADRTYCFNGFSGCTFAAETGIALYSLHDLWPADVAEAMARHGMTRLYAALHLPPEVLLPPGTYHTTSYLLIHDGDRAVITYEGDSSAGYNHDVSILRAWIRTTKVTGDHPLVIERVRAVGCHFVLLLTAAPEPSPMPYVPYPRSTEVYVRSIFGPGGSPSLFPTACSTKSTFHAVPVHIWDRLMLFGATLDDQAFCCSRLMTYLRGISYKVTVGALVANEGWNASEDALTAVITAAYLTICHQRYLRTQAISKGMKRLELEHAQKFITRLYSWLFEKSGRDYIPGRQLQFYAQCRRWLSAGFHLDPRVLVFDEAAPCRCRSLLRKAAHKFCCFMRWLGQDCTCFLQPVEGRVGEQGYDNEAFEGSDVDPAEEATVSISGSYIVTGSQLQPLYQALGIPSDLAARAGRLTATVEVSDTDGRLTCKTTMGNKTFTTVFTDGAQLEANGPEQYVLSFDLAKQTMAAGPHSLSYALTPAGLEVRVVSAGLDCRAVFPSGVATPSASGEVSAFCSALYRFNRCVQRHSLIGGLWYYPEGLIGLFPPFAPGHTWESANPFCGESTLYTRTWSVSGFSSCFSPFEPPALDSPPPAEADTPMAVDVPPPATLTLPQPPAPERAVPPQDLADGDVARASPGVSAAPPVPAQSVTDPPVSRRRLLHTYPDGSKVYAGSLFESECTWLVNASNPGHRPGGGLCHAFYQRFPESFDPAEFVMSDGFAAYTLTPRPIIHAVAPDYRVEHNPKRLEAAYRETCSRRGTAAYPLLGAGIYKVPVGLSFDAWERNHRPGDELYLTEPAIAWFEANRPTLPALTITEDTARTANLALELDSATEVGRACAGCRVEPGVVHYQFTAGVPGSGKSRSIQQGEVDVVVVPTRELRNSWRRRGFAAYTPHTAARVTRGRRVVIDEAPSLPPHLLLLHMQRASSVHLLGDPNQIPAIDFEHAGLVPAIRPELVPTKWWHLTHRCPADVCELIRGAYPKIQTASRVFRSLFWGEPPVGQKLVFTQAAKAANPGAITVHEAQGATFTETTIIATADARGLIQSSRAHAIVALTRHTEKCVVVDAPGLLREVGISDAIVNNFFLSGGQIGQHRPSVIPRGTINNNVDTLDAFPPSCQFSAYHQLAEELGHRPAPIAAVLPPCPELEQGLLYMPQELTTSDSVLTFELTDIVHCRMAAPSQRKAVLSTLVGRYGRRTKLYEAAHADVRGSLNHFIPELGPVSVTTCELYELVEAMVEKGQDGSAVLELDLCSRDVSRITFFQKDCNKFTTGETIAHGKVGQGISAWSKTFCALFGPWFRAIEKEILAVLAPNVFYGDAYEDTVLAAAVAGAPGCKVFENDFSEFDSTQNNFSLGLECIIMEECGMPQWMIRLYHLVRSAWVLQAPKESLRGFWKKHSGEPGTLLWNTVWNMAVIAHCYEFRDLKVAAFKGDDSVVLCSDYRQSRDAAALIAGCGLKLKVDFRPIGLYAGVVVAPGLGTLPDVVRFAGRLSEKNWGPGSERAEQLRLAVCDFLRKLTNVAQVCVDVVSQVYGVSPGLVHNLIGMLQTIADGKAHFTETVKPVLDLTNSIIHRVE

SEQ ID NO: 55 - protein, 674 aa, Hepatitis E virus JKK-Sap (ORF-2)
MNNMFFCSVHGDATMRSRAFLFLFLVLLPMLPAPPAGQPSGRRRGRRSGGAGGGFWGDRVDSQPFALPYIHPTNPFASDIPAAAGAGARPRQPARPLGSAWRDQSQRPATSARRRSAPAGASPLTAVAPAPDTAPVPDVDSRGAILRRQYNLSTSPLTSTIATGTNLVLYAAPLSPLLPLQDGTNTHIMATEASNYAQYRVVRATIRYRPLVPNAVGGYAISISFWPQTTTTPTSVDMNSITSTDVRILVQPGIASELVIPSERLHYRNQGWRSVETSGVAEEEATSGLVMLCIHGSPVNSYTNTPYTGALGLLDFALELEFRNLTPGNTNTRVSRYSSSARHKLRRGPDGTAELTTTAATRFMKDLHFTGTNGVGEVGRGIALTLFNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQTTYGSSTNPMYVSDTVTFVNVATGAQGVSRSLDWSKVTLDGRPLMTIQQYSKTFFVLPLRGKLSFWEAGTTKAGYPYNYNTTASDQILIENAAGHRVCISTYTTNLGSGPVSISAVGVLAPHSALAALEDTVDYPARAHTFDDFCPECRALGLQGCAFQSTVAELQRLKMKVGKTREY

SEQ ID NO: 56 - protein, 1707 aa, Hepatitis E virus JAK-Sai (ORF-1)
MEAHQFIKAPGVTTAIEQAALAAANSALANAVVVRPFLSRLQTEILINLMQPRQLVFRPEVLWNHPIQRVIHNELEQYCRARAGRCLEVGAHPRSINDNPNVLHRCFLKPVGRDVQRWYTAPTRGPAANCRRSALRGLPPADRTYCFDGFSGCTFAAETGVALYSLHDLWPADVAEAMARHGMTRLYAALHLPPEVLLPPGTYHTTSYLLIHDGDRAVITYEGDSSAGYNHDVSILRAWIRTTKVTGDHPLVIERVRAVGCHFVLLLTAAPEPSPMPYVPYPRSTEVYVRSIFGPGGSPSLFPSACSTKSTFHAVPVHIWDRLMLFGATLDDQAFCCSRLMTYLRGISYKVTVGALVANEGWNASEDALTAVITAAYLTICHQRYLRTQAISKGMKRLELEHAQKFITRLYSWLFEKSGRDYIPGRQLQFYAQCRRWLSAGFHLDPRVLVFDEAAPCRCRSLLRKAAHKFCCFMRWLGQDCTCFLQPIEGRVGEQGYDNEAFEGSDVDPAEEATVSISGSYIVTGSQLQPLYQALGIPSDLAARAGRLTATVEVSDADGRLTCKTIMGNKTFTTVFTDGAQLEVNGPEQYVLSFDLAKQTMAAGPHSLSYVLTSAGLEVRVVSAGLDCKAVFPSGVATPSASGEVSAFCSALYRFNRCVQRHSLIGGLWYYPEGLIGLFPPFAPGHTWESTNPFCGESTLYTRTWSVSGFSSCFSPLEPPASGSLPPAEIDPPVTVDAPSPSILALPRPSVFEQTTPPLDPAGDAAASAPPGAPGVPPAPARPVTHPSGPRRRLLHTYPDGSKVYAGSLFESECTWLVNASNPGHRPGGGLCHAFYQRFPESFDPAEFVMSDGFAAYTLTPRPIIHAVAPDYRVEHNPKRLEAAYRETCSRRGTAAYPLLGAGIYKVPVGLSFDAWERNHRPGDELYLTEPAIAWFEANRPTLPALTITEDTARTANLALELDSATEVGRACAGCRVEPGVVHYQFTAGVPGSGKSRSIQQGEVDVVVVPTRELRNSWRRRGFAAYTPHTAARVTRGRRIVIDEAPALPPHLLLLHMQRASSVHLLGDPNQIPAIDFEHAGLVPAIRPELVPTKWWHLTHRCPADVCELIRGAYPKIQTASRVLRSLFWGEPPVGQKLVFTQAAKAANPGAITVHEAQGATFTETTIIATADARGLIQSSRAHAIVALTRHTEKCVVVDAPGLLREVGISDAIVNNFFLSGGQIGQHRPSVIPRGTVDSNVDTLDAFPPSCQFSAYHQLAEELGHRPAPIAAVLPPCPELEQGLLYMPQELTTSDSVLTFELTDIVHCRMAAPSQRKAVLSTLVGRYGRRTKLYEAAHADVRGSLNHFIPELGPISVTTCELYELVEAMVEKGQDGSAVLELDLCSRDVSRITFFQKDCNKFTTGETIAHGKVGQGISAWSKTFCALFGPWFRAIEKEILAALAPNVFYGDAYEDTVLAAAVAGAPGCKVFENDFSEFDSTQNNFSLGLECIIMEECGMPQWMIRLYHLVRSAWVLQAPKESLRGFWKKHSGEPGTLLWNTVWNMAVIAHCYEFRDLKVAAFKGDDSVVLCSDYRQSRNAAALITGCGLKLKVDFRPIGLYAGVVVAPGLGTLPDVVRFAGRLSEKNWGPGPERAEQLRLAVCDFLRKLTNVAQVCVDVVSRVYGVSPGLVHNLIGMLQTIADGKAHFTETVKPVLDLTNSIIHRVE

SEQ ID NO: 57 - protein, 674 aa, Hepatitis E virus JAK-Sai (ORF-2)
MNNMFFCSVHGDATMRSRALLFLLFVFLPMLPAPPAGQPSGRRRGRRSGGAGGGFWGDRVDSQPFALPYIHPTNPFASDIPTAAGAGARPRQPARPLGSAWRDQSQRPTASARRRPAPAGAAPLTAVAPAPDTAPVPDVDSRGAILRRQYNLSTSPLTSTIATGTNLVLYAAPLSPLLPLQDGTNTHIMATEASNYAQYRVVRATIRYRPLVPNAVGGYAISISFWPQTTTTPTSVDMNSITSTDVRILVQPGIASELVIPSERLHYRNQGWRSVETSGVAEEEATSGLVMLCIHGSPVNSYTNTPYTGALGLLDFALELEFRNLTPGNTNTRVSRYSSSARHKLRRGPDGTAELTTTAATRFMKDLHFTGTNGVGEVGRGIALTLFNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQTTYGSSTNPMYVSDTVTFVNVATGAQGVSRSLDWSKVTLDGRPLTTIQQYSKTFFVLPLRGKLSFWEAGTTKAGYPYNYNTTASDQILIENAAGHRVCISTYTTNLGSGPVSISAVGVLAPHSALAALEDTVDYPARAHTFDDFCPECRTLGLQGCAFQSTVAELQRLKMKVGKTREY

SEQ ID NO: 58 - DNA, 19 bp, Artificial
  Synthetic DNA
tcgatgccatggaggccca

SEQ ID NO: 59 - DNA, 19 bp, Artificial
  Synthetic DNA
gccytkgcgaatgctgtgg

SEQ ID NO: 60 - DNA, 20 bp, Artificial
  Synthetic DNA
tcraarcagtargtgcggtc

SEQ ID NO: 61 - DNA, 20 bp, Artificial
  Synthetic DNA
catagcctcsgcracatcag

SEQ ID NO: 62 - DNA, 20 bp, Artificial
  Synthetic DNA
catygcctcsgcaacatcgg

SEQ ID NO: 63 - DNA, 20 bp, Artificial
  Synthetic DNA
tyaaaacagtaggttcgatc

SEQ ID NO: 64 - DNA, 52 bp, Artificial
  Synthetic DNA
aaggctcctggcrtyactactgcyatwgagcaggcwgctctrgcwgcggcaa

SEQ ID NO: 65 - DNA, 19 bp, Artificial
  Synthetic DNA
ctcctggcatcactactgc

SEQ ID NO: 66 - DNA, 19 bp, Artificial
  Synthetic DNA
ctcctggcattactactgc

SEQ ID NO: 67 - DNA, 18 bp, Artificial
  Synthetic DNA
ctcctggcgtcactactg

SEQ ID NO: 68 - DNA, 14 bp, Artificial
  Synthetic DNA
gcgaatgctgtggt

SEQ ID NO: 69 - DNA, 19 bp, Artificial
  Synthetic DNA
gccggtcggtagggacgtc

SEQ ID NO: 70 - DNA, 24 bp, Artificial
  Synthetic DNA
gggttatacacaatgaattagaac

(19) United States
(12) Patent Application Publication
(10) Pub. No.: US 2006/0228702 A1
(43) Pub. Date: Oct. 12, 2006

(54) Polynucleotide probe and primer derived from hepatitis E virus recovered from japanese, chip including the same, kit including the same, and method of detecting hepatitis E virus genome using the same

(76) Inventors: Kazuaki Takahashi, Kanagawa (JP); Shunji Mishiro, Tokyo (JP); Yasuhiko Oota, Tokyo (JP); Michie Hashimoto, Tokyo (JP); Hiroshi Maekubo, Hokkaido (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(21) Appl. No.: 11/455,703

(22) Filed: Jun. 20, 2006

Related U.S. Application Data
(62) Division of application No. 10/239,090, filed on Sep. 19, 2002.

(30) Foreign Application Priority Data
Jun. 25, 2001 (JP) 2001-191837

(51) Int. Cl. C12Q 1/70; C12Q 1/68; C07H 21/04; C12M 1/34

(52) U.S. Cl. 435/5; 435/6; 536/24.1; 435/287.2

(57) ABSTRACT

A polynucleotide probe that has a sequence of at least 8 nucleotides and is used for detecting at least one hepatitis E virus selected from JSN-FH, JKN-Sap, JMY-Haw, JAK-Sai, and JRA1; a probe assay kit that includes the polynucleotide probe; and a chip on which the polynucleotide probe has been solid-phase fixed.

### CROSS REFERENCE TO RELATED APPLICATIONS

This application is a continuation application of U.S. application Ser. No. 10/239,090. This application claims priority to, and incorporates herein by reference, U.S. application Ser. No. 10/239,090, International Application PCT/JP02/06365, and Japanese Application 2001-191837.

### TECHNICAL FIELD

The present invention relates to a novel method for detecting hepatitis E virus. The present invention also relates to a novel strain of hepatitis E virus recovered from Japanese, a novel strain of hepatitis E virus from a patient with fulminant hepatitis, and polynucleotide derived therefrom, which is important for establishing the novel method for detecting the RNA genome of hepatitis E virus.

### Background Art

Hepatitis E virus (which will be referred to as “HEV” hereinafter) which replicates in the liver of a patient is voided to feces rather than staying in blood. Accordingly, HEV is transmitted mainly by feco-oral route. Thus, HEV infection sometimes happens as a local outbreak caused by contamination of a water system. Due to such a manner of infection, hepatitis E caused by HEV is frequently observed in regions where the sanitary environment is not satisfactory, such as Asia and Africa. On the contrary, HEV infection is relatively rare in the industrially advanced countries such as Japan, US and Europe. Most of the hepatitis E cases, which are occasionally reported in these advanced countries, are found in travelers who have been to the regions where the disease is endemic and recently come back to their own countries. Accordingly, hepatitis E is generally recognized in the advanced countries as an “imported disease”.

HEV was genetically cloned by Reyes of Genelabs Co., in the United States, in 1990, for the first time. The entire nucleotide sequence of the HEV genome was then revealed by the study thereafter. The HEV strains whose full-genome sequence had been analyzed were what is called the “Mexico” strain and the “Burma” strain. Thereafter, the sequences of the genomes derived from the “India” strain, the “Pakistan” strain, the “Nepal” strain, the “Burma” strain, the “China” strain, the “US” strain and the like have been sequentially been revealed. However, the nucleotide sequence of the HEV gene derived from the “Japan” strain has not been revealed yet.

The conventional method of diagnosing HEV infection includes: a method of conducting a PCR method by using a primer designed on the basis of the nucleotide sequences of the aforementioned various known HEV strains, for detecting the RNA genome of the virus; and a method of using, as an antigen, a peptide/protein which has been synthesized/expressed on the basis of the amino acid sequence of the known HEV strains, for detecting an antibody specific thereto.

In the conventional method or technique of diagnosing HEV infection, if an unknown HEV strain having a line different from the known HEV strains exists in the sample to be tested, there is a significant possibility that the unknown virus cannot be detected.

### DISCLOSURE OF INVENTION

An object of the present invention is to provide a method which enables “wide-range” detection of HEV belonging to various strains. Another object of the present invention is to provide polynucleotide for widely detecting HEV belonging to various strains and polynucleotide for determining the strain of the detected HEV.

Yet another object of the present invention is to provide polynucleotide derived from a novel HEV strain endemic to Japan, polynucleotide derived from a novel strain of fulminant HEV, and polypeptides coded by these nucleotide sequences.

A further object of the present invention is to provide a method for carrying out a drug design, which drug design is achieved by utilizing the genetic information of the novel HEV described above and the genetic information of the conventional known HEV.

The inventors of the present invention have molecularly isolated, for the first time in the world, the HEV strains (specifically the HEV Japan JRA1 strain, the JKN-Sap strain, the JMY-Haw strain, JKK-Sap strain and JAK-Sai strain) from Japanese patients, isolated HEV Japan JSN-FH strain as a novel HEV strain from a case of fulminant hepatitis, and determined the genome sequence of the obtained virus. The present invention has been achieved on the basis of these discoveries.

According to a first aspect of the present invention, there is provided a polynucleotide probe including a sequence comprising at least eight nucleotides, the polynucleotide probe being used for detecting the genomic polynucleotide of hepatitis E virus, characterized in that:

(1) the sequence comprising at least eight nucleotides is hybridized with the polynucleotide of the hepatitis E virus, thereby, due to the hybridization, detecting the hepatitis E virus; and

(2) the sequence comprising at least eight nucleotides is obtained from a sequence selected from the group consisting of nucleotide sequences disclosed at SEQ No. 11, SEQ No. 44, SEQ No. 45, SEQ No. 46, SEQ No. 47 and SEQ No. 48 and complementary strands thereof.

According to a second aspect of the present invention, there is provided a pair or plural pairs of primer for PCR for amplifying polynucleotide of hepatitis E virus, the at least a pair of primer for PCR each independently having a sequence comprising at least eight nucleotides, characterized in that:

(1) the sequence comprising at least eight nucleotides is hybridized with the genomic polynucleotide of the hepatitis E virus, thereby, due to the hybridization, amplifying a portion of the polynucleotide of the hepatitis E virus; and

(2) the sequence comprising at least eight nucleotides is obtained from a sequence selected from the group consisting of nucleotide sequences disclosed at SEQ No. 11, SEQ No. 44, SEQ No. 45, SEQ No. 46, SEQ No. 47 and SEQ No. 48 and complementary strands thereof.

According to a third aspect of the present invention, there is provided a method of detecting presence of hepatitis E virus in a sample, comprising:

(1) obtaining a sample from an object;

(2) reacting the sample obtained in the obtaining of (1) with the polynucleotide probe according to the first aspect;

(3) detecting a double strand produced as a result of the reaction of the reacting of (2);

(4) determining whether or not hepatitis E virus is present in the sample, on the basis of the detection result of the detecting of (3).

According to a fourth aspect of the present invention, there is provided a method for detecting presence of hepatitis E virus in a sample, comprising:

(1) obtaining a sample from an object;

(2) reacting the sample obtained in the obtaining of (1) with a pair of primer for PCR according to the second aspect and polymerase, under a condition in which amplification is effected in an appropriate manner;

(3) detecting presence of a product obtained as a result of amplification by the reaction of the reacting of (2);

(4) determining whether or not hepatitis E virus is present in the sample, on the basis of the detection result of the detecting of (3).

According to a fifth aspect of the present invention, there is provided a method for determining genotype of hepatitis E virus in a sample, comprising:

(1) reacting a sample with a pair of primer for PCR according to claim 12 and polymerase, under a condition in which amplification is effected in an appropriate manner;

(2) determining length of a product obtained as a result of amplification by the reaction of the reacting of (1);

(3) determining genotype of hepatitis E virus present in the sample, on the basis of the detection result of the determining of (2).

According to a sixth aspect of the present invention, there is provided a probe assay kit, including the polynucleotide probe according to the first aspect.

According to a seventh aspect of the present invention, there is provided a PCR assay kit, including the pair or plural pairs of primer for PCR according to the second aspect.

According to an eighth aspect of the present invention, there is provided a chip for detecting a nucleotide sequence, on which the polynucleotide probe according to the first aspect has been immobilized.

### BRIEF DESCRIPTION OF DRAWINGS

FIG. 1 is a view showing a phylogenetic tree of a hepatitis E virus strain.

FIG. 2 is a view showing a genetic organization of HEV JRA1.

FIGS. 3A-3F are views showing a portion of the nucleotide sequence of HEV JRA1 and a portion of the nucleotide sequence of the “Mexico” strain in a manner that the former is compared with the latter.

FIG. 4 is a view showing a primer and a probe according to embodiments of the present invention.

FIGS. 5A-5F show a portion of the nucleotide sequence of HEV JSN-FH and a portion of the nucleotide sequence of the “Mexico” strain in a manner that the former is compared with the latter.

FIG. 6 is a view showing the ORF regions of various HEV strains in a manner that the ORF regions are compared with one another.

FIG. 7 is a view showing a phylogenetic tree of the HEV strain.

FIGS. 8A - 8D show the sequence of the ORF1 of various HEV strains in a manner that the ORF1 regions are compared with one another.

FIG. 9 is a view showing the data which indicates the presence of RNA of HEV in a serum sample collected from a patient, which data is shown combined with changes in the liver function test values.

FIG. 10 is a chart showing results of HEV detection, obtained from samples of patients of non A, B, C acute hepatitis by using the primer according to embodiments of the present invention.

FIG. 11 is a chart showing results of HEV detection, which was successively conducted for a patient, according to embodiments of the present invention.

FIG. 12 is a view showing a result of detection of the amplified HEV genome by electrophoresis, according to embodiments of the present invention.

FIGS. 13A to 13D are views showing the relationship between the types of a probe, the genotype of HEV and absorbance.

### BEST MODE FOR CARRYING OUT OF THE INVENTION

I. Terms

The term “polynucleotide” used in the present specification inclusively represents, for convenience, polynucleotide, oligonucleotide and the like. Further, the term “polynucleotide” used in the present specification represents a substance resulted from phosphoric ester-bonding of no less than two nucleoside. Nucleoside generally includes deoxyribonucleoside and ribonucleoside, without being limited thereto. “Oligonucleotide” represents a substance obtained by polymerizing, by means of phosphodiester bonding, phosphoric esters of a few to dozens of nucleoside (i.e., nucleotide). Oligonucleotide generally includes oligoribonucleotide and oligodeoxyribonucleotide, without being limited thereto. The polynucleotide according to embodiments of the present invention may be either virus-genome RNA recovered from the HEV JRA1 strain or DNA (or the like) obtained from the virus-genome RNA. Further, “polynucleotide” of the present invention may include artificially synthesized nucleic acid such as peptide nucleic acid, morpholino nucleic acid, methylphosphonate nucleic acid and S-oligo nucleic acid.

The “polypeptide” used in the present specification generally represents a peptide composed of no less than two amino acids, an oligopeptide or a protein. The “polypeptide” used in the present specification includes both synthesized peptide and expressed protein, without being limited thereto. The “polypeptide” may be either a simple protein composed of only amino acids or a composite protein containing components other than amino acids.

The term “object” used in the present specification may be one of any mammal or the like, including human, dog, cat, cow, goat, pig, sheep and monkey. In the present embodiment, a human is the most suitable object.

The term “sample” used in the present specification represents a biosample such as blood, serum, stool, liver and lymph, collected from an individual or biont as the object. The “sample” of the present invention may also be obtained by subjecting a biosample to any necessary preparatory treatment such as homogenization, extraction or the like. Such a preparatory treatment will be easily selected by one skilled in the art, in accordance with the biosample used as the object.

The term “open reading frame” used in the present specification represents a region of a polynucleotide sequence which codes a polypeptide. This region represents at least a portion of the coding sequence.

The term “hepatitis E virus” used in the present specification represents the virus as the primary cause of epidemic non-A, non-B hepatitis, which spreads by the medium of drinking water, is transmitted by way of digestive tract and is observed mainly in Asia and Africa. The hepatitis E virus is RNA virus and classified into genus calicivirus of the family caliciviridae. The term “HEV” used in the present specification comprehensively represents a virus belonging to the genotypes I, II, III and IV, commonly used for virus classification. Further, according to embodiments the present invention, any strain which belongs to HEV and has not been isolated/identified can be detected and/or identified or classified into a genotype. Accordingly, the term “HEV” used in the present specification can include an unknown HEV and a virus which is detected and/or identified or classified into a genotype according to embodiments the present invention. “HEV” in the present specification includes a wild type, a variant, and a strain which is genetically close to HEV. Specifically, “HEV” in the present specification represents a strain which shows homology of approximately 50% or more, preferably 60% or more, and most preferably 65% or more, with respect to the sequence of approximately 2500 nucleotides on the 5′ end side of JRA1 shown in Sequence No. 1.

II. Strain of Novel Hepatitis E Virus

The inventors of the present invention have assumed that an HEV strain which is indigenous to Japan does exist, on the basis of the fact that hepatitis E cases have been observed in Japan even among those who have never been abroad, although the number of these cases is relatively small. Under this assumption, the inventors of the present invention have attempted cloning of HEV genomes recovered from Japanese patients who developed acute hepatitis and, as a result, succeeded in isolating five HEV strains which appear to belong to a novel line.

These novel HEV strains are five strains named as follows by the inventors: HEV Japan JRA1 strain (Hepatitis E virus Japan JRA1, also referred to as “JRA1 strain” hereinafter, NCBI accession No. AP003430); HEV Japan JKN-Sap strain (Hepatitis E virus Japan JKN-Sap, also referred to as “JKN-Sap strain” hereinafter, NCBI accession No. AB074918); HEV Japan JMY-Haw strain (Hepatitis E virus Japan JMY-Haw, also referred to as “JMY-Haw strain” hereinafter, NCBI accession No. AB074920); HEV Japan JKK-Sap strain (Hepatitis E virus Japan JKK-Sap, also referred to as “JKK-Sap strain” hereinafter, NCBI accession No. AB074917); and HEV Japan JAK-Sai strain (Hepatitis E virus Japan JAK-Sai, also referred to as “JAK-Sai strain” hereinafter, NCBI accession No. AB074915). With regards to the origin of each virus strain, JKK-Sap, JKN-Sap and JMY-Haw were derived from acute hepatitis E patients from Hokkaido, JAK-Sai is derived from an acute hepatitis E patient from Saitama prefecture, and JRA1 is derived from a patient from Tokyo.

Further, the inventors of the present invention have cloned the HEV JSN-FH strain (Hepatitis E virus JSN-FH, which will occasionally be referred to as “JSN-FH strain”) as a novel HEV strain from a fulminant hepatitis case.

Open reading frame 1 (which will be referred to as “ORF1”hereinafter) of the genome RNA sequence of JRA1 strain is shown at the sequence (SEQ) No. 1, and the whole length of this genome RNA sequence is shown at the SEQ No. 48.

The semi-whole length of the genome RNA sequence of JSN-FH strain, which does not include a portion of non-coding sequence at the 5′ end side of the genome RNA sequence, is shown at the SEQ No. 11. The amino acid sequences which are coded in ORF1 and ORF 2 of the genome RNA sequence of JSN-FH strain are shown at the SEQ No. 12 and the SEQ No. 13, respectively.

A portion of ORF1 of the genome RNA sequence of each of JKN-Sap strain, JMY-Haw strain, JAK-Sai strain and JKK-Sap strain is shown at the SEQ No. 16, the SEQ No. 17, the SEQ No. 20 and the SEQ No. 21, respectively. Further, the whole length of the genome RNA sequence of each of JKN-Sap strain, JMY-Haw strain, JAK-Sai strain and JKK-Sap strain is shown at the SEQ No. 44, the SEQ No. 45 (the semi-whole length), the SEQ No. 46 (the semi-whole length) and the SEQ No. 47 (the semi-whole length), respectively.

2. HEV JRA1 Strain

(1) HEV JRA1 Strain

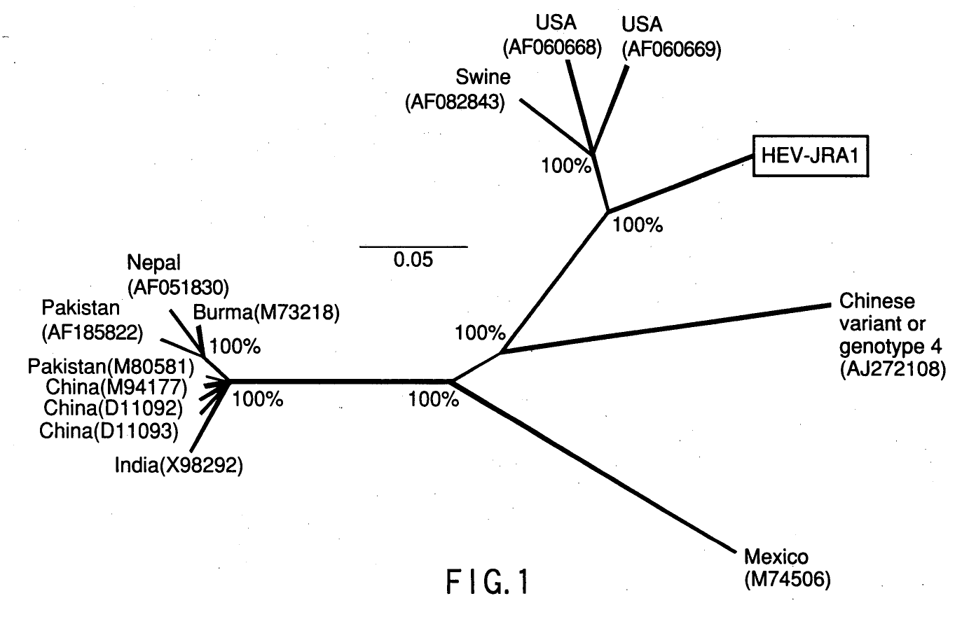

The inventors of the present invention have revealed that the nucleotide sequence of JRA1 strain disclosed at the SEQ No. 1 belongs to a novel genotype, as compared with the known strains of various types collected from various areas in the world, as shown in the phylogenetic tree of FIG. 1.

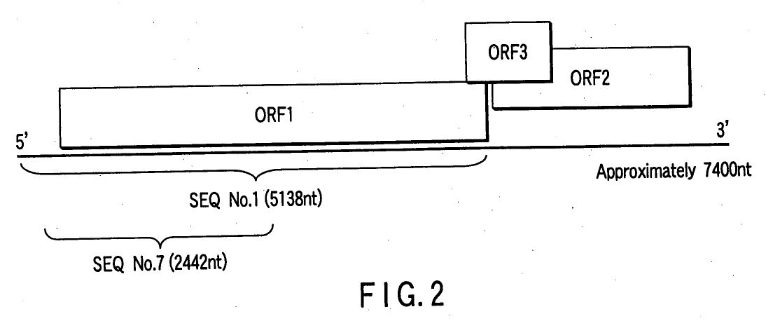

The difference in nucleotide sequence between the known strains and JRA1 strain is particularly obvious in the approximately 2500 nucleotides at the genome 5′ end. As shown in FIG. 2, the hepatitis E virus genome, including HEV-JRA1, generally contains three open reading frames (which will be referred to as “ORF” hereinafter) of ORF1, ORF2 and ORF3. The approximately 5000 nucleotides at the 5′ end is included to ORF1. The genome regions at the downstream side of ORF1, i.e., the other regions including the ORF2 region and the ORF3 region exhibit relatively high sequence-conservation between strains, as compared with the ORF1 region.

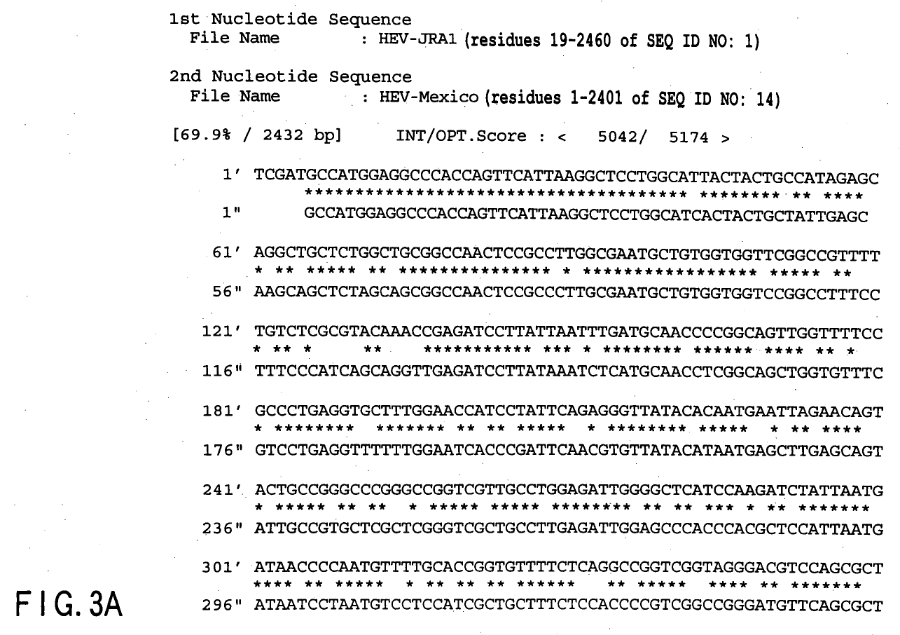

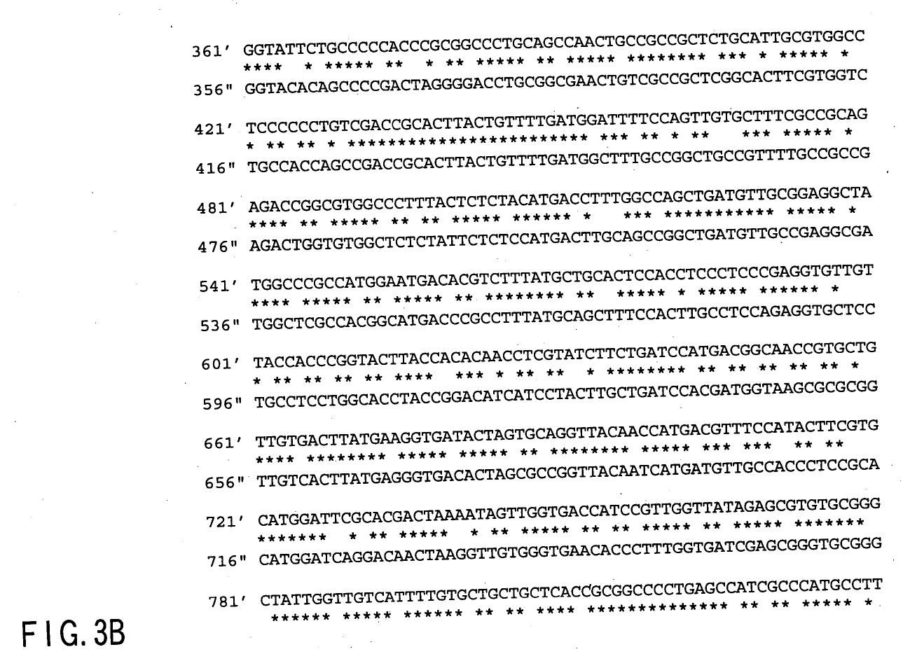

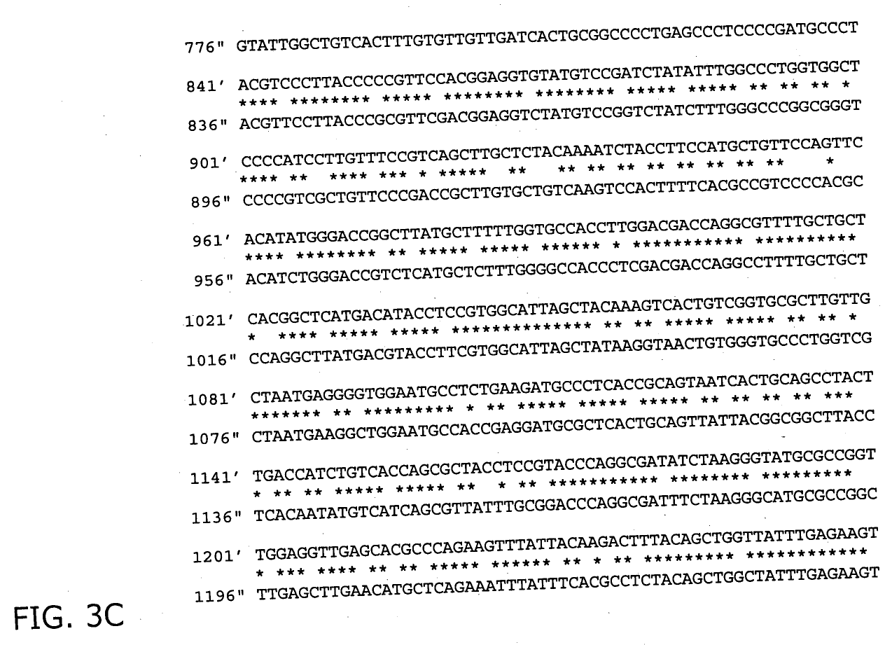

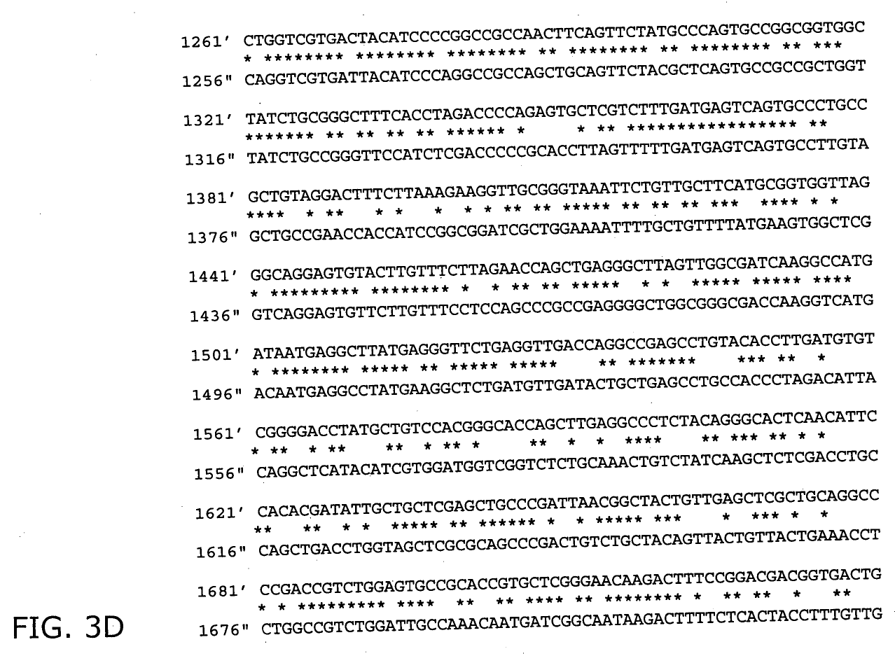

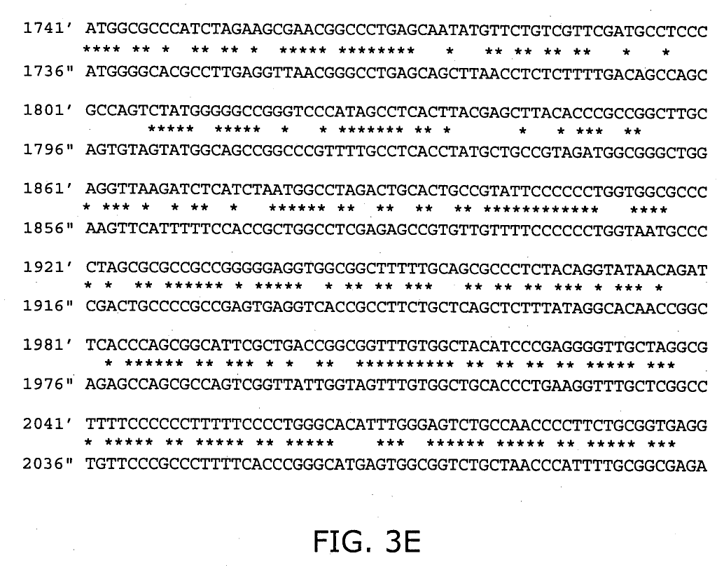

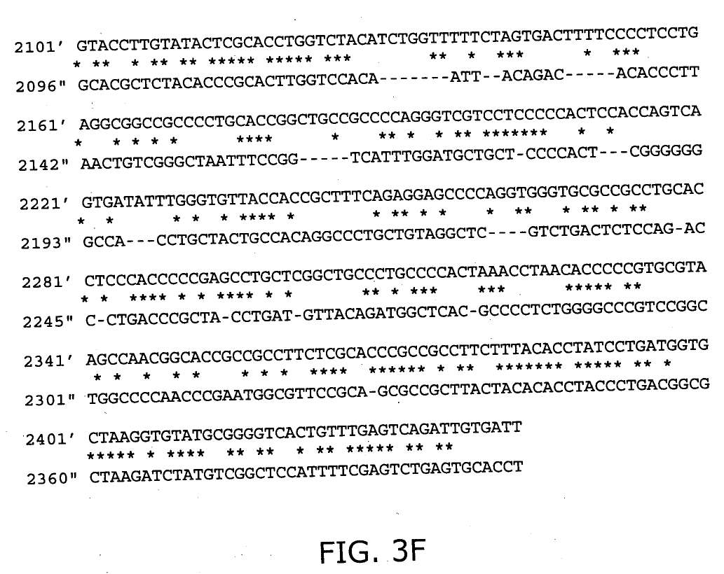

The sequence of the approximately 2500 nucleotides at the 5′ end, in particular, in the ORF1 region exhibits, regarding the portion of the nucleotide sequence of JRA1 strain shown at the SEQ No. 7 of the sequence list described below, degree of homology (of sequence) of only 70% or less, as compared with the “Mexico” strain (SEQ ID NO-10 of USP 5,789,559, NCBI accession No. M74506, shown at the SEQ No. 49) which still shows the highest homology to JRA1 among the nucleotide sequence disclosed in the patents owned by Reyes. The nucleotide sequences of JRA1 strain and the “Mexico” strain are shown in FIGS. 3A-3F in a manner that the former is compared with the latter. In FIGS. 3A-3F , the sequence of HEV-JRA1 strain and a portion of the sequence of the “Mexico” strain are shown in a juxtaposed manner so that the former is shown as the upper sequence and the latter is shown as the lower sequence. The homology between the two sequences is 69.9% per 2432 nucleotides.

Accordingly, as such a specific novel sequence as described above of JERA1 strain has been revealed, JRA1 strain, which has never been subjected to detection and thus never been properly detected, can now be detected in an easy and accurate manner. Further, it is expected that the strains which are genetically close to JRA1 strain, which have hardly been detected by the conventional method, can also be detected by the present invention. Description will now be given of the embodiments of the present invention regarding the determination of the novel genome sequence of the novel hepatitis E virus strain described above.

(2) Polynucleotide

In one embodiment of the present invention, a polynucleotide which is specific to JRA1 strain and has a novel sequence is provided. The polynucleotide of the present embodiment may be, for example, a polynucleotide shown as a nucleotide sequence of the SEQ No. 1 containing 5138 nucleotides. Alternatively, the polynucleotide may be a polynucleotide fragment, as a nucleotide sequence of any of the regions of the SEQ No. 1 nucleotide sequence. Specifically, the polynucleotide may be a polynucleotide shown as the nucleotide sequence of SEQ No. 7 containing 2442 nucleotides, or, a complementary strand of any one of the nucleotide sequences described above. Or, the polynucleotide of the present embodiment may be a polynucleotide fragment as a nucleotide sequence which constitutes a portion of any one of the aforementioned polynucleotides and the complementary strands thereof.

One or a few nucleotides of each polynucleotide described above may be subjected to modification such as deletion, substitution or addition.

In another embodiment of the present invention, a primer and a probe each constituted of any one of the nucleotide sequences of the SEQ Nos. 1 to 7 or the complementary strand thereof or a polynucleotide fragment having a fragment of any one of the nucleotide sequences of the SEQ Nos. 1 to 7 and the complementary strand thereof, are provided. The primer and probe may be used as a primer for amplification for detecting HEV in the detection object (for example, as a primer for PCR amplification or the like), or as a probe used in a “DNA chip”. In this case, the number of nucleotides contained in the polynucleotide of the present embodiment is preferably in a range of 10 to 30 (inclusive of both 10 and 30). If the length of the polynucleotide fragment is too long, it is difficult to recognize the difference between the fragments, as the difference could be one nucleotide. If the length of the polynucleotide is too short, it is difficult to determine the nucleotide sequence of polynucleotide contained in the sample.

Examples of the primer and the probe of the present embodiment as described above include those for comprehensively detecting HEV and for selectively detecting each genotype of HEV.

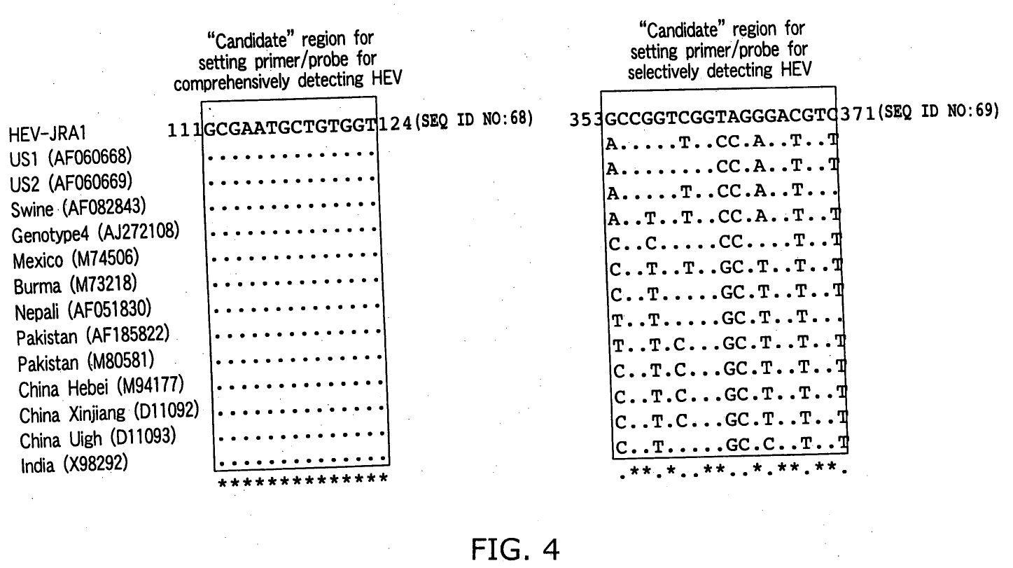

FIG. 4 shows a portion of the nucleotide sequence information of JRA1 strain and the nucleotide sequence information of known strains at the corresponding site, in a juxtaposed manner, so that the former is compared with the latter. By comparing the nucleotide sequence of JRA1 strain with the nucleotide sequence of known strains at the corresponding site, as shown in the example of FIG. 4, it is possible to clearly identify a highly-conserved region useful for setting a primer/probe for comprehensively detecting HEV and a highly-mutated region for selectively detecting each genotype of HEV.

The term “highly-conserved region” used in the present invention indicates a region which exhibits high homology, e.g., 90% or more, preferably 95% or more, and most preferably 100% of homology between the nucleotide sequence of HEV-JRA1 strain and the corresponding nucleotide sequence of a known strain. The term “highly-mutated region” used in the present specification indicates a region which is a nucleotide sequence specific to HEV-JRA1 strain and exhibits relatively low homology, e.g., 80% or less, preferably 75% or less, and most preferably 70% or less of homology between the nucleotide sequence of HEV-JRA1 strain and the corresponding nucleotide sequence of a known strain.

FIG. 4 shows the 111^{th} nucleotide to the 124^{th} nucleotide of the SEQ No. 1 of HEV-JRA1 strain as an example of a “candidate” region for setting a primer and a probe for comprehensively detecting HEV, and the region corresponding thereto of known strains. The polynucleotide shown as the nucleotide sequences of the SEQ Nos. 2 to 5 or the complementary strand of any one of the nucleotide sequences of the SEQ Nos. 2 to 5, for example, can be used as a primer and a probe for comprehensively detecting HEV. On the other hand, the region of the SEQ No. 1 which can be used as a primer and a probe for comprehensively detecting HEV is not limited thereto.

Similarly, FIG. 4 shows the 353^{rd} nucleotide to the 371^{st} nucleotide of the SEQ No. 1 of JRA1 strain as an example of a “candidate” region for setting a primer and a probe for selectively detecting HEV, and the region corresponding thereto of known strains. The polynucleotide shown as the nucleotide sequences of the SEQ Nos. 1 to 7 or the complementary strand of any one of the nucleotide sequences of the SEQ Nos. 1 and 7 (specifically, the polynucleotide including the nucleotide sequence of the SEQ No. 6 or the complementary strand of the nucleotide sequence of the SEQ No. 6), for example, can be used as a primer and a probe for selectively detecting HEV. On the other hand, the region of the SEQ Nos. 1 and 7 which can be used as a primer and a probe for selectively detecting HEV is not limited thereto.

In a further embodiment of the present invention, a polynucleotide including, as a portion thereof, the polynucleotide of the SEQ Nos. 1 to 7 or a fragment of the polynucleotide of the SEQ Nos. 1 to 7 is provided. The polynucleotide of the present embodiment may be a polynucleotide including a specific polynucleotide, which specific polynucleotide is obtained as a result of bonding of at least one type of a polynucleotide selected from the group consisting of: genes of a promoter, an enhancer, an upstream activation sequence, a silencer, an upstream repression sequence, an attenuator, poly(A) tail, nucleus transition signal, ISRE, a drug resistance factor, and a signal-peptide gene; gene of a membrane-penetration region; and gene of a marker protein including luciferin, green fluorescent protein, phycocyanin and horseradish peroxidase. Alternatively, the polynucleotide of the present embodiment may be a polynucleotide including any other suitable nucleotide sequence.

(3) Polypeptide

In another embodiment of the present invention, a polypeptide as the amino acid sequence disclosed at the SEQ No. 8 is provided. The inventors of the present invention have demonstrated, by the clinical study, that the aforementioned polypeptide of the present embodiment is a polypeptide produced at an early stage in a HEV-JRA1 patient and that the antibody specific to the polypeptide is an antibody produced at an early stage in a HEV-JRA1 patient. Accordingly, the polypeptide and/or the antibody of the present embodiment can be used as a marker for HEV-JRA1 detection. The polypeptide and the antibody as described above can be obtained by the known methods. A polypeptide obtained as a result of modification such as deletion, substitution, addition of one or a few peptides, of a fragment of the aforementioned polypeptide, a polypeptide including the fragment, and an amino acid sequence disclosed at the SEQ No. 8, is also included within the scope of the present invention.

According to the present embodiment, if the polypeptide is a synthesized peptide, for example, and when the polypeptide is used as a polypeptide for antibody detection in which an antibody is detected as a result of bonding of the antibody to the polypeptide, the polypeptide of the present embodiment may include the polypeptide shown as the sequential 15 to 50 amino acid residue contained in the SEQ No. 8. If the polypeptide of the present embodiment is an expressed protein, the protein may include the polypeptide shown as the sequential 150 to 250 amino acid residue contained in the SEQ No. 8.

3. HEV Japan JSN-FH Strain

(1) HEV Japan JSN-FH Strain

As described above, JSN-FH strain is a novel HEV strain isolated from a patient of a fulminant hepatitis case. The method of medical treatment is completely different between fulminant hepatitis and ordinary acute hepatitis. Accordingly, if the disease is diagnosed as fulminant hepatitis at an early stage, in other words, if the diagnosis as fulminant hepatitis can be made soon after the infection, the possibility of survival increases. However, the amount of HEV virus expression in a fulminant hepatitis patient is extremely small. Therefore, there were previously scant reports that HEV was successfully recovered from a fulminant hepatitis patient. Needless to say, determining the sequence over the whole length of HEV has been regarded virtually impossible. However, the inventors of the present invention have succeeded in molecular cloning of HEV from a fulminant hepatitis patient and also in determining the approximate whole length (or the approximate semi-whole length) of the genome of the HEV.

The genome of HEV recovered from a fulminant hepatitis patient had a unique sequence, as a whole. In one of the clones obtained by sub-cloning, a new point mutation was found. The mutation produces a premature stop codon in an open reading frame (ORF2) coding nucleocapsid protein and shortens the length of the translated product, from 660 amino acids which would be the length if ORF2 of the normal hepatitis E virus strain were to effect coding, to 211 amino acids. In short, the 212^{th} codon of ORF2 was replaced with any one of the stop codons TAA, TAG and TGA.

For hepatitis B, it has been found that, when a patient has been infected with a variant (strain) in which the translated product coded by the pre-core/core region cannot reach the full size, the patient is likely to develop fulminant hepatitis.

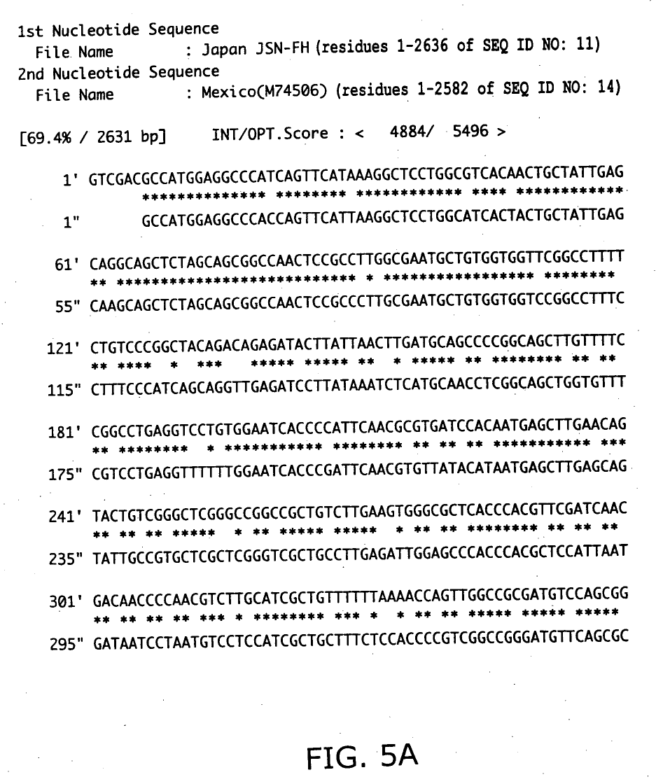

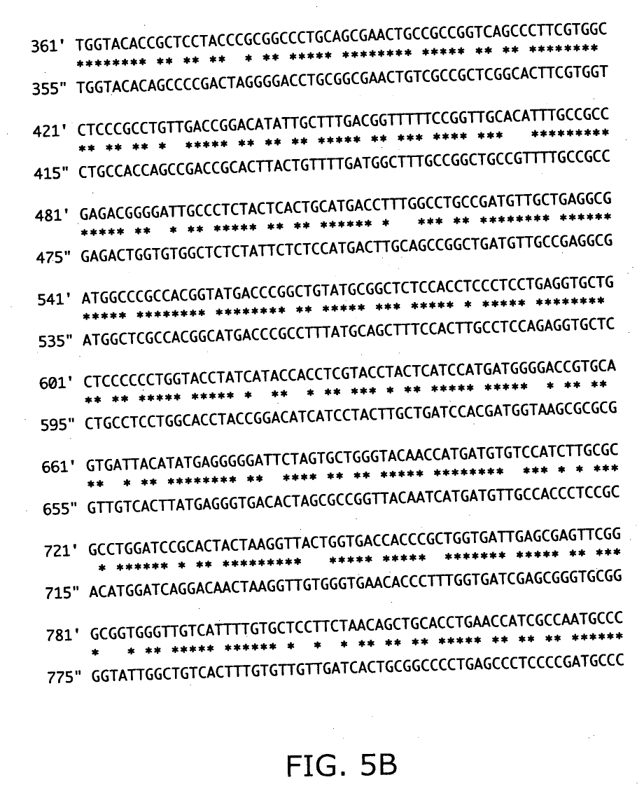

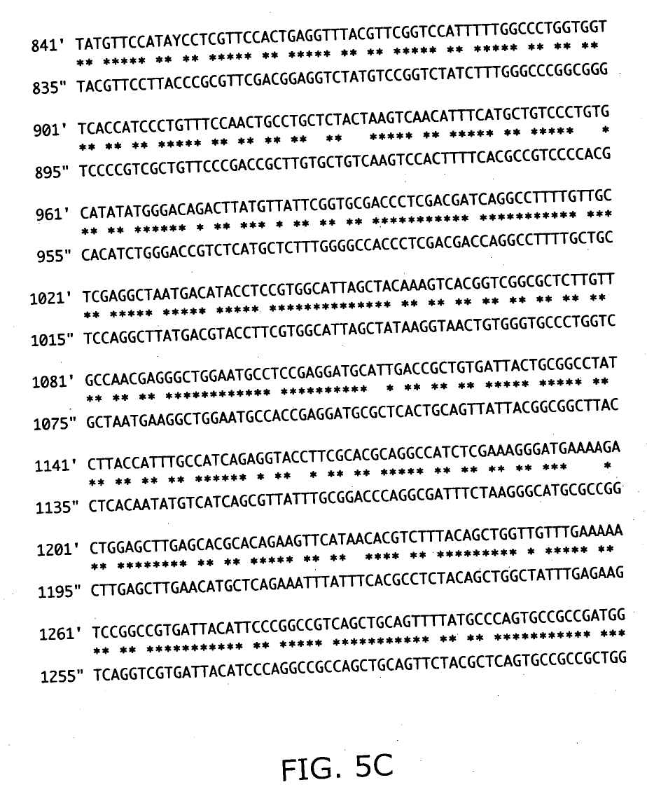

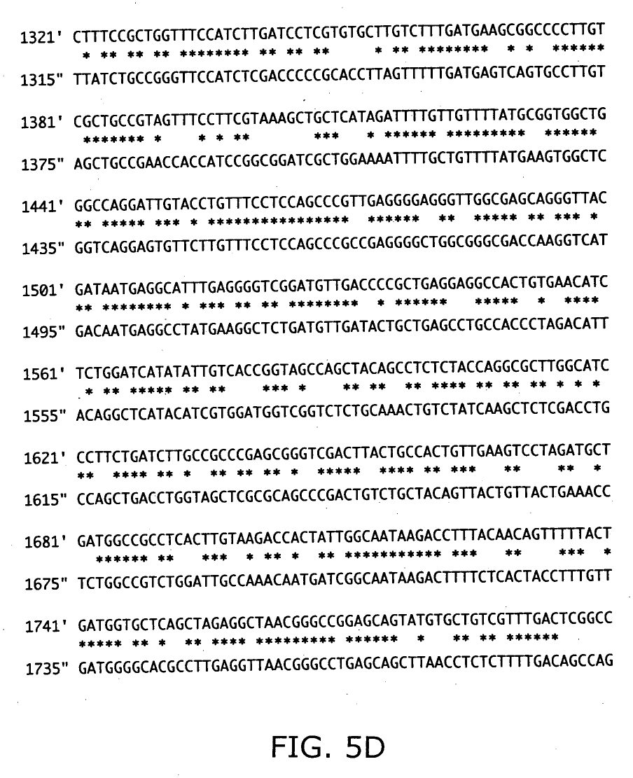

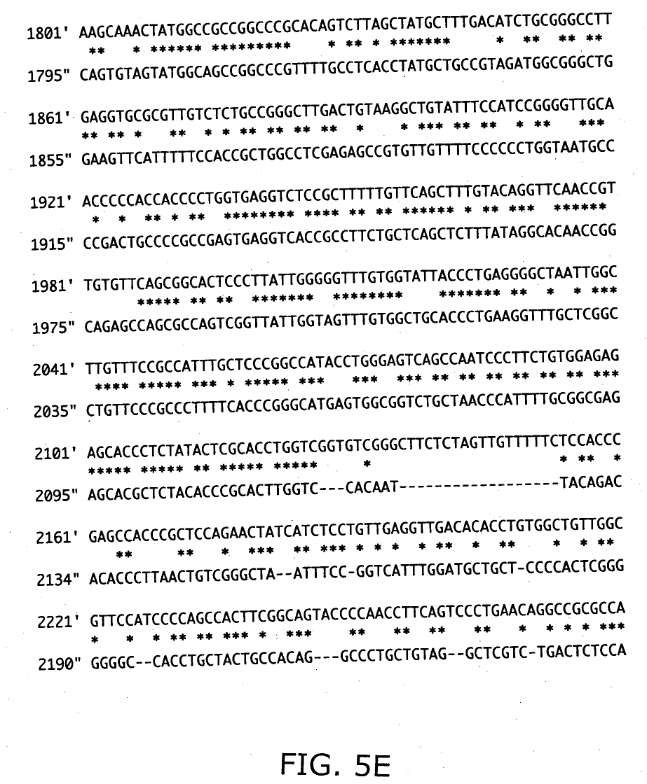

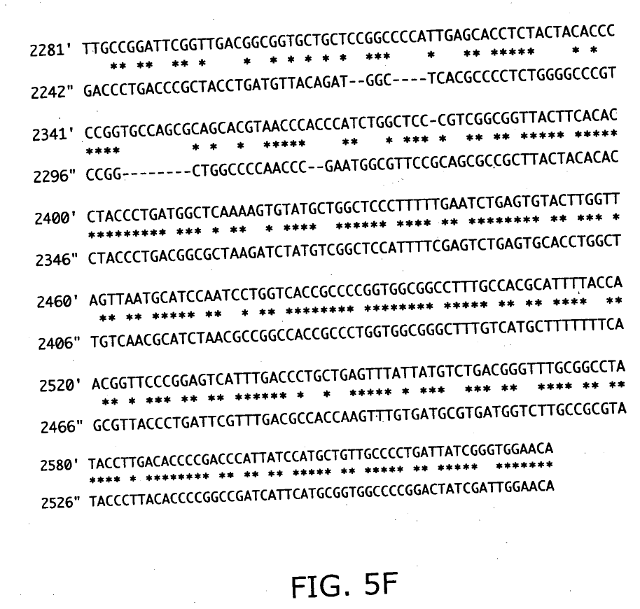

The nucleotide sequence of JSN-FH strain and the nucleotide sequence of “Mexico” strain (M74506) are shown in FIGS. 5A-5F in a manner that the former is compared with the latter. The nucleotide sequence of HEV JSN-FH strain and the nucleotide sequence of “Mexico” strain (M74506) are shown in a juxtaposed manner, such that the nucleotide sequence of JSN-FH strain is located upper and the corresponding nucleotide sequence of “Mexico” strain (M74506) is located therebelow. In this case, the rate of homology is 69.4% per 2631 nucleotides.

Accordingly, as such a specific novel sequence as described above of JSN-FH strain has been revealed, JSN-FH strain, which has never been subjected to detection and thus never been properly detected, can now be detected in an easy and accurate manner. Further, it is expected that the strains which are genetically close to JSN-FH strain, which had not been detected by the conventional method, can also be detected by the present invention. If JSN-FH strain is detected in a patient, the result indicates that the patient is likely to develop fulminant hepatitis.

(2) Polynucleotide

In a further embodiment of the present invention, a polynucleotide which is specific to JSN-FH strain and has a novel sequence is provided. The polynucleotide of the present embodiment may be, for example, a polynucleotide shown as a nucleotide sequence of SEQ No. 11 containing 7234 nucleotides. Alternatively, the polynucleotide may be a polynucleotide fragment, as a nucleotide sequence of any of the regions of the SEQ No. 11 nucleotide sequence. Specifically, the polynucleotide may be a polynucleotide shown as the nucleotide sequence of SEQ No. 22 containing 238 nucleotides. Or, the polynucleotide of the present embodiment may be a complementary strand of any one of the nucleotide sequences described above. Or, the polynucleotide of the present embodiment may be a polynucleotide fragment, as a nucleotide sequence which constitutes a portion of any one of the aforementioned polynucleotide and the complementary strand thereof.

Further, as the polynucleotide for determining whether or not the strain has a stop codon in ORF2, polynucleotides having the nucleotide sequences disclosed at the SEQ Nos. 9 and 10, and complementary strands of the polynucleotides having the nucleotide sequences disclosed at the SEQ Nos. 9 and 10, and a polynucleotide fragment having a fragment of any one of the aforementioned polynucleotides or the complementary strands thereof, may be provided. A primer and a probe may be provided using the polynucleotide described above. The nucleotide sequence disclosed at the SEQ Nos. 9 and 10 each represents a nucleotide sequence of a portion of ORF2 of JSN-FH strain. The nucleotide sequence disclosed at the SEQ No. 9 represents a nucleotide sequence which allows specifically detecting a strain not having a stop codon. The nucleotide sequence disclosed at the SEQ No. 10 represents a nucleotide sequence which allows specifically detecting a strain having a stop codon. A primer including a nucleotide sequence for amplifying a nucleotide portion including the aforementioned portion, also falls within the scope of the present invention. By detecting a nucleotide sequence which does not allow production of the full-size product of translation in ORF2 of the nucleic acid contained in the sample collected from the object, and detecting, on the basis of the aforementioned detection, that the object has been infected with a virus strain having such a stop codon in ORF2, it is possible to diagnose, at an early stage of infection, that the object or the patient has been infected with fulminant hepatitis virus.

One or a few nucleotides of each polynucleotide described above may be modified by, for example, deletion, substitution or addition.

In another embodiment of the present invention, a primer and a probe each constituted of any one of the nucleotide sequences disclosed at the SEQ Nos. 11 and 22 or the complementary strand thereof or a polynucleotide fragment having a fragment of any one of the nucleotide sequences of the SEQ Nos. 11 and 22 and the complementary strands thereof, are provided. The primer and probe may be used as a primer for various amplification for detecting HEV in the detection object (for example, as a primer for PCR amplification or the like), or as a probe used in a “DNA chip”. In this case, the number of nucleotides contained in the polynucleotide of the present embodiment is preferably in a range of 10 to 30, inclusive. As previously mentioned, if the length of the polynucleotide fragment is too long, it will be difficult to recognize the difference between the fragments, and the difference could be one nucleotide. If the length of the polynucleotide is too short, it will generally be difficult to determine the nucleotide sequence of polynucleotide contained in the sample.

According to the present embodiment, polynucleotides of the SEQ Nos. 11, 22, 9 and 10 or a polynucleotide including, as a portion thereof, a fragment of any one of the polynucleotides of the SEQ Nos. 11, 22, 9 and 10 is provided. The polynucleotide of the present embodiment may be a polynucleotide including a specific polynucleotide, and this polynucleotide is obtained as a result of bonding of at least one type of polynucleotide selected from the group consisting of: genes of a promoter, an enhancer, an upstream activation sequence, a silencer, an upstream repression sequence, an attenuator, poly(A) tail, nucleus transition signal, ISRE, a drug resistance factor, and a signal-peptide gene; gene of a membrane-penetration region; and gene of a marker protein including luciferin, green fluorescent protein, phycocyanin and/or horseradish peroxidase. Alternatively, the polynucleotide of the present embodiment may be a polynucleotide including any other suitable nucleotide sequence.

(3) Polypeptide

Further, in yet another embodiment of the present invention, polypeptides as the amino acid sequences disclosed at the SEQ Nos. 12 and 13 are provided. The amino acid sequence disclosed at the SEQ No. 12 is an amino acid sequence corresponding to the region of ORF1 of the genome sequence of JSN-FH strain. The amino acid sequence disclosed at the SEQ No. 13 is an amino acid sequence corresponding to the region of ORF2 of the genome sequence of JSN-FH strain.

The polypeptide and/or the antibody of the present embodiment can be used as a marker for JSN-FH detection. The polypeptide and the antibody as described above can be obtained by known methods. A polypeptide obtained as a result of modification such as deletion, substitution, addition of one or a few peptides, of a fragment of the aforementioned polypeptide, a polypeptide including the fragment, and an amino acid sequence disclosed at the SEQ No. 12 or No. 13, is also included within the scope of the present invention.

According to the present embodiment, if the polypeptide is a synthesized peptide, for example, and when the polypeptide is used as a polypeptide for antibody detection in which an antibody is detected as a result of bonding of the antibody to the polypeptide, the polypeptide of the present embodiment may include the polypeptide shown as the sequential 15 to 50 amino acid residue contained in the SEQ No. 12 or No. 13. If the polypeptide of the present embodiment is an expressed protein, the protein may include the polypeptide shown as the sequential 150 to 250 amino acid residue contained in the SEQ No. 12 or No. 13.

ORF1, which is represented by the SEQ No. 12, is a region coding various enzyme proteins necessary for replication of virion or other purposes. Accordingly, in addition to the data of the primary structure i.e., the SEQ No. 12, the data of the secondary structure and/or the tertiary structure of the protein(s) may be further obtained by known methods, such as X-ray diffraction, so that drug design can be carried out on the basis of the obtained data. Such drug design itself and a medicine obtained from the drug design are also included within the scope of the present invention.

An antibody specific to the shortened nucleocapsid protein, derived from ORF2 represented by the SEQ No. 13, may be produced by the known methods. By utilizing an antigen-antibody reaction in which the antibody as described above is used, HEV infection can be easily diagnosed, using a sample collected from the object.

An antibody for recognizing the antibody specific to the nucleocapside protein may be produced-by a known method. By utilizing such an antibody, HEV infection can be easily diagnosed, using a sample collected from the object.

4. Comparison of the Novel HEV Strain With the Conventional HEV Strain

(1) Comparison of the Novel HEV Strain With the Conventional HEV Strain

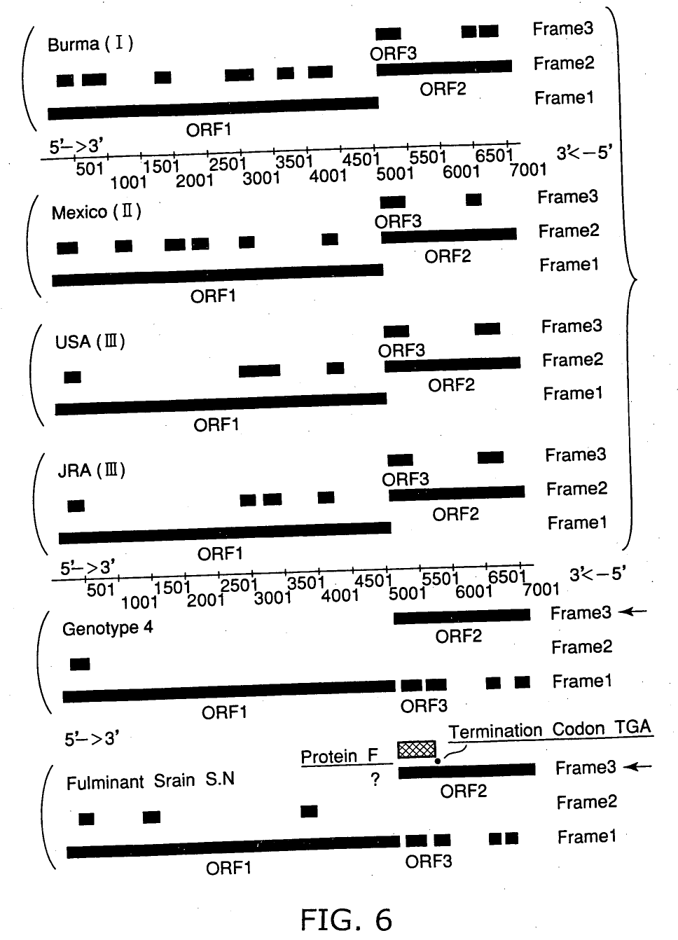

FIG. 6 shows the results of comparison in which HEV Burma B1 strain (M73218), HEV Mexico strain (M74506), HEV USA US-1 strain (AF060669), HEV Japan JRA1 (AP003430), Genotype 4 and HEV Japan JSN-FH are compared with each other, with regards to the location of ORF in each of the genome sequence thereof. The number in parenthesis added after the name of each strain is the NCBI accession number. Note that, in the present invention, “HEV” of each strain name may omitted (for example, HEV USA US-1 strain may read “USA US-1 strain”)

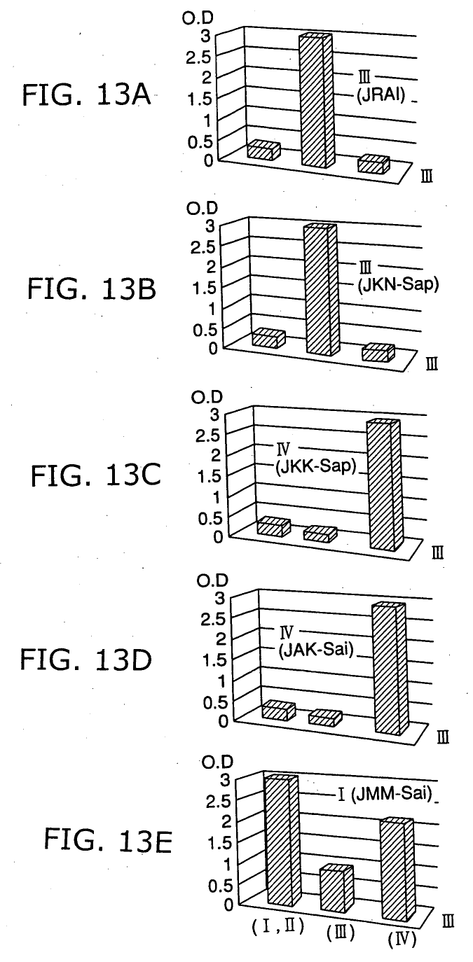

The Roman numerals shown in parenthesis, next to each strain name, represent the genotype to which each strain belongs. As shown in FIG. 13, a termination codon TGA is present in OFR2 of JSN-FH strain.

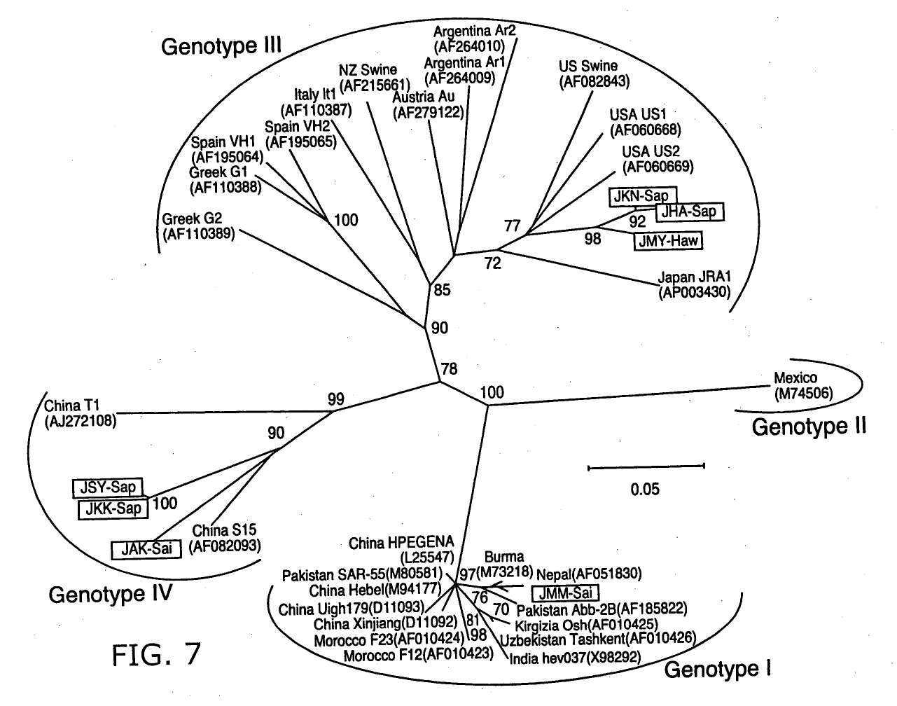

FIG. 7 shows a phylogenetic tree produced (according to the neighbor-joining method) on the basis of the 326nt region of ORF1, which represents genealogy of the strains of 7 types endemic to Japan, including the novel strains of 5 types of the present invention, and the HEV strains found in the countries other than Japan. As shown in FIG. 7, JMM-Sai was classified into type I. JKN-Sap, JHA-Sap and JMY-Haw were each classified into type III. JSY-Sap, JKK-Sap and JAK-Sai were each classified into type IV.

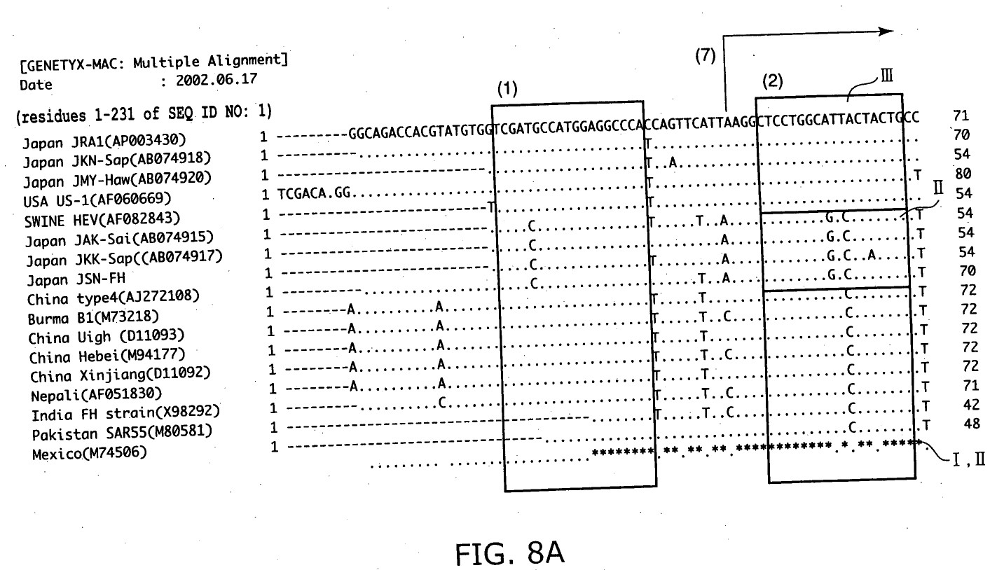

In FIGS. 8A - 8D, the information of the nucleotide sequence of the ORF1 region of the following examples is shown, in a juxtaposed manner, for comparison. The examples include: the strains endemic to Japan of 5 types, as one embodiment of the present invention, i.e., {Japan JRA1 strain (SEQ No. 15), JKN-Sap strain (SEQ No. 16), JMY-Haw strain (SEQ No. 17), JKK-Sap strain (SEQ No. 21) and JAK-Sai strain (SEQ No. 20)}; the strain derived from a fulminant hepatitis patient (JSN-FH strain, SEQ No. 22); and the known foreign strains i.e., {USA US-1 (AF060669, SEQ No. 18), SWINE HEV (AF082843, SEQ No. 19), China type4 (AJ272108, SEQ No. 23), Burma B1 (M73218, SEQ No. 24), China Uigh (D11093, SEQ No. 25), China Hebei (M94177, SEQ No. 26), China Xinjiang (D11092, SEQ No. 27), Nepali (AF051830, SEQ No. 28), India FH strain (X98292, SEQ No. 29), Pakistan SAR55 (M80581, SEQ No. 30), and Mexico (M74506, SEQ No. 31)}.

In FIGS. 8A-8D, the strain names, the NCBI accession No. shown in parenthesis next to the strain names, and the information of nucleotide sequence next to the NCBI accession No. are shown in the aforementioned order. The numbers shown at both ends of the sequence each represent the number of nucleotide, counted from the transcript-initiation site whose number is “1”. JRA 1 strain, shown as the uppermost sequence in FIGS. 8A-8D, is regarded as the reference. When the type of the nucleotide at a specific position of another strain coincides with the type of the nucleotide of JRA1 strain at the corresponding position, the nucleotide of the former is indicated by the symbol “.”. When the type of the nucleotide at a specific position of another strain differs from the type of the nucleotide of JRA1 strain at the corresponding position, the type of the nucleotide of the former is identified as it is. At the lowermost sequence in FIGS. 8A-8D, the degree of conservation of sequence at the plurality of nucleotide sequences shown in the juxtaposed manner is indicated. Specifically, the symbol “*” at the lowermost sequence indicates that the type of the nucleotide at the specific position is the same throughout the plurality of strains. On the other hand, the symbol “.” at the lowermost sequence indicates that type of the nucleotide at the specific position of the sequence is different between JRA1 strain and non-JRA1 strains, in at least one strain of the latter.

All of the novel strains and the nucleotide sequences thereof described above are included within the scope of the present invention.

(2) Polynucleotide

According to a further embodiment of the present invention, polynucleotides which are specific to the strains JKN-Sap, JMY-Haw, JKK-Sap and JAK-Sai, respectively, and each have a novel sequence, are provided. The nucleotide sequence of the present embodiment may be, for example, a polynucleotide shown as a nucleotide sequence of any of the SEQ No. 44, the SEQ No. 45, the SEQ No. 46 and the SEQ No. 47, which nucleotide sequences represent the genome sequence of JKN-Sap, JMY-Haw, JKK-Sap and JAK-Sai, respectively. Alternatively, the polynucleotide may be the complementary strand of each of the nucleotide sequences described above. Yet alternatively, the polynucleotide may be a polynucleotide fragment, which constitutes a portion of any one of the aforementioned polynucleotides and the polynucleotides as the complementary strands thereof.

The polynucleotide of the present embodiment may be a polynucleotide fragment shown as the nucleotide sequence of any of the regions of the polynucleotide. The polynucleotide of the present invention may be a polynucleotide shown as a nucleotide sequence of any of the SEQ No. 16, the SEQ No. 17, the SEQ No. 20 and the SEQ No. 21 coding the ORF1 region. Or, the polynucleotide of the present embodiment may be a complementary strand of any one of the nucleotide sequences described above. Or, the polynucleotide of the present embodiment may be a polynucleotide fragment, which constitutes a portion of any one of the aforementioned polynucleotides and the complementary strands thereof.

Further, a primer including a nucleotide sequence for amplifying the nucleotide portion, including the nucleotide sequences of the SEQ No. 16, the SEQ No. 17, the SEQ No. 20 and the SEQ No. 21, is included within the scope of the present invention.

One or a few peptides of each polynucleotide described above may be subjected to modification such as deletion, substitution and addition.

In a further embodiment of the present invention, a primer and a probe each constituted of any one of the nucleotide sequences of the SEQ No. 44, the SEQ No. 45, the SEQ No. 46 and the SEQ No. 47 or the complementary strands thereof or a polynucleotide fragment having a fragment of any one of the nucleotide sequences of the SEQ Nos. 44 to 47 and the complementary strands thereof, are provided. The primer and probe may be used as a primer for various amplification for detecting HEV in the detection object (for example, as a primer for PCR amplification or the like), or as a probe used in a “DNA chip”. As previously mentioned number of nucleotide contained in the polynucleotide of the present embodiment is preferably in a range of 10 to 30, inclusive. If the length of the polynucleotide fragment is too long, it will be difficult to recognize the difference between the fragments, and the difference could be one nucleotide. If the length of the polynucleotide is too short, it will be difficult to determine the nucleotide sequence of polynucleotide contained in the sample.

Further, another embodiment of the present invention, a polynucleotide including, as a portion thereof, the polynucleotide of any of the SEQ No. 44, the SEQ No. 45, the SEQ No. 46 and the SEQ No. 47, as well as the SEQ No. 16, the SEQ No. 17, the SEQ No. 20 and the SEQ No. 21 or a polynucleotide having, as a portion thereof, a fragment of these polynucleotides is provided. The polynucleotide of the present embodiment may be a polynucleotide including a specific polynucleotide, obtained as a result of bonding of at least one type of polynucleotide selected from the group consisting of: genes of a promoter, an enhancer, an upstream activation sequence, a silencer, an upstream repression sequence, an attenuator, poly(A) tail, nucleus transition signal, ISRE, a drug resistance factor, and a signal-peptide gene; gene of a membrane-penetration region; and gene of a marker protein including luciferin, green fluorescent protein, phycocyanin and horse radish peroxidase. Alternatively, the polynucleotide of the present embodiment may include any other suitable nucleotide sequence.

(3) Polypeptide

In another embodiment of the present invention, polypeptides shown as the amino acid sequences coded by the polynucleotide sequence disclosed at the SEQ No. 44, the SEQ No. 45, the SEQ No. 46 and the SEQ No. 47, as well as the SEQ No. 16, the SEQ No. 17, the SEQ No. 20 and the SEQ No. 21, which are specific to the strains of JKN-Sap, JMY-Haw, JKK-Sap and JAK-Sai, respectively, are provided. Further, the present embodiment provides polypeptides and the fragments thereof, and the polypeptides are shown by the amino acid sequences of the SEQ No. 50, the SEQ No. 52, the SEQ No. 54 and the SEQ No. 56 representing the amino acid sequences of the ORF1 region and the SEQ No. 51, the SEQ No. 53, the SEQ No. 55 and the SEQ No. 57 representing the amino acid sequences of the ORF2 region, specific to the strains of JKN-Sap, JMY-Haw, JKK-Sap and JAK-Sai, respectively.

The polypeptide derived from the amino acid sequence coded by the ORF1 region can be utilized in carrying out drug design.

The polypeptide derived from the amino acid sequence coded by the ORF2 region can be used for producing an antibody for detecting the respective strains. Alternatively, the polypeptide derived from the amino acid sequence coded by the ORF2 region can, by itself, be used as an antibody for detection of diagnostic purpose. Or, vaccine may be produced by utilizing this polypeptide.

According to the present embodiment, if the polypeptide is a synthesized peptide, for example, and when the polypeptide is used as a polypeptide for antibody detection in which an antibody is detected as a result of bonding of the antibody to the polypeptide, the polypeptide of the present embodiment may include the amino acid sequence shown by the ORF2 region of the nucleotide sequence derived from the respective novel strains, e.g., the polypeptide shown as the sequential 15 to 50 amino acid residues contained in the SEQ No. 51, the SEQ No. 53, No. 55 or the SEQ No. 57. If the polypeptide of the present embodiment is an expressed protein, the protein may include the polypeptide shown as the sequential 150 to 250 amino acid residue contained in the SEQ No. 51, the SEQ No. 53, No. 55 or the SEQ No. 57.

The polypeptide and/or the antibody described above can also be used as a marker for detecting HEV JKN-Sap, JMY-Haw, JKK-Sap and JAK-Sai, respectively. The polypeptide and the antibody as described above can be obtained by the known methods. A polypeptide obtained as a result of modification such as deletion, substitution, addition of one or a few peptides, of a fragment of the aforementioned polypeptide, a polypeptide including the fragment, and an amino acid sequence disclosed at the SEQ Nos. 50 to 57, is also included within the scope of the present invention.

According to the present embodiment, if the polypeptide is a synthesized peptide, for example, and when the polypeptide is used as a polypeptide for antibody detection in which an antibody is detected as a result of bonding of the antibody to the polypeptide, the present polypeptide may include the polypeptide shown as the sequential 15 to 50 amino acid residues contained in the SEQ No. 8. If the polypeptide of the present embodiment is an expressed protein, the protein may include the polypeptide shown as the sequential 150 to 250 amino acid residues contained in the SEQ No. 8.

5. Primer and Probe for Comprehensively Detecting HEV

As a result of the aforementioned discovery of the novel HEV strains and the determination of the nucleotide sequence thereof, a primer which can amplify the HEV genome of a variety of types (including unknown types) all at a time, that is, a universal primer (i.e., a primer for comprehensive HEV detection) can be provided. Further, a probe which can detect HEV of a variety of types (including unknown types) all at a time (i.e., a probe for comprehensive HEV detection) can be provided. Such a universal primer will be described hereinafter.

In FIGS. 8A-8D, the nucleotide sequences of ORF1 of the representative HEV strains, each of which belongs to Group I, II, III or IV, are listed. In the tables of FIGS. 8A-8D, the symbol shown inside the parenthesis next to the strain name is the NCBI accession No. The numbers shown at both ends of the sequence each represent the number of the nucleotide, counted from the transcript-initiation site whose number is “1”.

The term “universal primer” or “universal probe” represents a polynucleotide fragment containing nucleic acid including a nucleotide sequence which allows comprehensive detection of HEV including unknown strains. For example, the nucleotide sequence of such a universal primer can be selected from the sequences shown in FIGS. 8A-8D. The sequence is, for example, preferably a highly conserved region.

The term “highly-conserved region” used in the present invention indicates a region which exhibits high homology, e.g., 90% or more, preferably 95% or more, and most preferably 100% of homology between the nucleotide sequence of HEV-JRA1 strain and the corresponding nucleotide sequence of a known strain.

FIGS. 8A-8D show an example in which a portion of the information of nucleotide sequence of JSN-FH strain is compared with the information of nucleotide sequence of known strains at the corresponding site. By comparing the nucleotide sequence of JSN-FH strain with the nucleotide sequence of the known strains in such a manner, it is possible to clearly identify a highly-conserved region useful for setting a primer/probe for comprehensively detecting HEV and a highly-mutated region for selectively detecting each genotype of HEV.

FIGS. 8A-8D show an example of a “candidate” region for setting a primer and a probe for comprehensively detecting HEV. Preferable examples of the highly conserved region include: the region indicated by “(1)” in FIG. 8A, i.e., the range from the 19^{th} (nucleotide) to the 37^{th} (nucleotide) of the SEQ No. 15 of HEV-JRA1 strain and the corresponding region of the known strains; the region indicated by “(4)” in FIG. 8B, i.e., the range from the 111^{th} to the 127^{th} of the SEQ No. 15 of HEV-JRA1 strain and the corresponding region of the known strains; the region indicated by “(5)” in FIG. 8C, i.e., the range from the 174^{th} to the 181^{st} of the SEQ No. 15 of HEV-JRA1 strain and the corresponding region of the known strains; and the region indicated by “(6)” in FIG. 8C, i.e., the range from the 213^{th} to the 220^{th} of the SEQ No. 15 of HEV-JRA1 strain and the corresponding region of the known strains.

Regarding the primer for comprehensively detecting HEV, use of a polynucleotide of preferably 6 to 100 nucleotides, more preferably 15 to 25 nucleotides having a sequence as included in the (1) region of FIG. 8A, as a sense primer, is preferable. Use of a polynucleotide of preferably 6 to 100 nucleotides, more preferably 15 to 25 nucleotides having a sequence as included in the (4), (5) and/or (6) regions of FIG. 8A, as a sense primer, together with the aforementioned sense primer, is more preferable.

The region indicated by “(7)” in FIGS. 8A-8D, i.e., the range from the 48^{th} to the 100^{th} of the SEQ No. 15 of HEV-JRA1 strain and the corresponding region of the known strains can also be used as the primer groups for comprehensively detecting HEV. In this case, the sequence extending over the entire length of the (7) region is not necessary and any suitable sequence of preferably 6 to 25 nucleotides, more preferably 15 to 22 nucleotides selected from the (7) region can be used. Preferable examples of the primer for comprehensively detecting HEV include:

| | |
|---|---|
| 5′-gcagaccacrtatgtgktcg-3′; | (SEQ No. 32) |
| 5′-ccacrtatgtggtcgaygcc-3′; | (SEQ No. 33) |
| 5′-acmarctgscgrggytgcat-3′; | (SEQ No. 34) |
| 5′-cgytgratwggrtgrttcca-3′; | (SEQ No. 35) |
| 5′-tgktcgaygccatggaggc-3′; | (SEQ No. 36) |
| 5′-tgktcgaygccatggaggc-3′; | (SEQ No. 37) |
| 5′-aygccatggaggcccaycag-3′; | (SEQ No. 38) |
| 5′-ckracyaccacagcattcgc-3′; | (SEQ No. 39) |
| and | |
| 5′-ggcckracyaccacagcatt-3′. | (SEQ No. 40) |

In the present specification, “a” or “A” represents adenine, “c” or “C” represents cytosine, “g” or “G” represents guanine, “t” or “T” represents thymine, “r” or “R” represents G or A. “y” or “Y” represents T or U or U. “w” or “W” represents A or T or U.

It is more preferable that the aforementioned primer groups for comprehensively detecting HEV is used, for example, for two-staged analysis as follows. In one example, the SEQ No. 32 and the SEQ No. 33 described above are used as sense primers and the SEQ No. 34 and the SEQ No. 35 described above are used as antisense primers, for the first amplification. Then, the SEQ No. 36, the SEQ No. 37 and the SEQ No. 38 described above are used as sense primers and the SEQ No. 39 and SEQ No. 40 described above are used as antisense primers, for the second amplification. As a result, polynucleotide fragments derived from unknown HEV variant strains of a variety of types can be obtained. The products obtained as a result of amplification may further be analyzed by known methods such as electrophoresis. The polynucleotide fragment which has been analyzed may then be classified into genotypes.

When the aforementioned sequences are used as probes, a polynucleotide of preferably 6 to 100 nucleotides, more preferably 12 to 25 nucleotides having a sequence including the sequences contained in the (1) region of FIG. 8A is preferably used as the probe for comprehensively detecting HEV. A polynucleotide of preferably 6 to 100 nucleotides, more preferably 12 to 25 nucleotides having a sequence including the sequences contained in the (4), (5) and/or (6) regions of FIG. 8A is more preferably used as the probe for this purpose.

It should be noted that the primer for comprehensively detecting HEV of the present invention is not restricted to the above-described preferable examples thereof.

6. Nucleotide Sequence for Genotype Identification

A primer and probe for comprehensively detecting HEV can be obtained by selecting the sequence of the highly conserved region as described above, and comprehensive virus detection is possible by using the obtained primer and probe. On the other hand, a primer and probe for selectively detecting HEV can be obtained by selecting the sequence of the highly mutated region. Selective detection and/or identification of virus can be possible by using such a highly mutated sequence.

The term “highly-mutated region” used in the present specification indicates a region which is a nucleotide sequence specific to HEV-JRA1 strain and exhibits relatively low homology, e.g., 80% or less, preferably 75% or less, and most preferably 70% or less of homology between the nucleotide sequence of HEV-JRA1 strain and the corresponding nucleotide sequence of known strains.

FIGS. 8A-8D show an example of a “candidate” region for setting a primer and a probe for selectively detecting HEV. Preferable examples of the highly mutated region include: the region indicated by “(2)” in FIG. 8A, i.e., the range from the 52^{nd} (nucleotide) to the 69^{th} (nucleotide) of the SEQ No. 15 of HEV-JRA1 strain and the corresponding region of the known strains; and the region indicated by “(3)” in FIG. 8B, i.e., the range from the 77^{th} to the 95^{th} of the SEQ No. 15 of HEV-JRA1 strain and the corresponding region of the known strains.

The (2) region of FIG. 8A is a region where the nucleotide sequence changes according to the genotype thereof. The (2) region of FIG. 8A has been demarcated to three portions according to the genotypes. Specifically, the strain included in the portion having the symbol “III” attached thereto in FIG. 8A belongs to genotype III. The strain included in the portion having the symbol “IV” attached thereto in FIG. 8A belongs to genotype IV. The portion having the symbol “I, II” attached thereto in FIG. 8A includes genotypes I or II. The (3) region of FIG. 8B can be understood in a manner similar to the (2) region of FIG. 8A. In short, the HEV to be analyzed can be classified into the respective genotypes, by utilizing the difference in type of nucleotide at a specific position of the sequence between the respective genotypes.

When the aforementioned sequences are used as probes, a polynucleotide of preferably 6 to 100 nucleotides, more preferably 12 to 25 nucleotides having a sequence including the sequences contained in the (2) region of FIG. 8A is preferably used as the probe for selectively detecting HEV. A polynucleotide of preferably 6 to 100 nucleotides, more preferably 12 to 25 nucleotides having a sequence including the sequence(s) contained in the (3) region of FIG. 8B is more preferably used as the probe for this purpose.

When the aforementioned sequences are used as primers, a polynucleotide of preferably 6 to 100 nucleotides, more preferably 12 to 25 nucleotides having a sequence including the sequences contained in the (2) region of FIG. 8A is preferably used as a primer for selectively detecting HEV. A polynucleotide of preferably 6 to 100 nucleotides, more preferably 12 to 25 nucleotides having a sequence including the sequence contained in the (3) region of FIG. 8B is more preferably used as the probe for this purpose. Or, a primer which is capable of amplifying polynucleotide including at least the (2) region of FIG. 8A and/or the (3) region of FIG. 8B may be selected.

5. Method of Detecting HEV Virus

In a further embodiment of the present invention, a method of detecting HEV virus in a sample is provided.

The term “sample” used in the present embodiment represents; a biosample including blood, serum, lymph, tissues, and excrement such as feces, urine or the like collected from a biont as the object; a untreated sample as an environmental sample e.g., water and soil collected from the environment including rivers, sewage and the like; and a sample obtained by subjecting the aforementioned biosample or the environment sample to any necessary preparatory treatment such as homogenation, extraction or the like. Such a preparatory treatment will be easily selected by one skilled in the art, in accordance with the biosample or the environmental sample used as the object.

According to the present embodiment, the method of detecting HEV virus can be carried out, by using the primer described above, according to the known amplification method such as the polym erase chain reaction (which reaction is generally called as “PCR” and thus will be referred to as “PCR” hereinafter). Typical examples of PCR includes reverse transcription PCR, reverse transcription nested PCR, or modification thereof such as reverse PCR, 5′ RACE and 3′ RACE.

The detection method as described above can be conducted, for example, as follows. First, a desired primer is mixed with a sample containing hepatitis E virus genome. A PCR reaction is carried out under an appropriate PCR condition in which, for example, the temperature is changed such that the initial 95° C. lasted for 4 minutes is followed by 30 cycles of {950° C. for 30 seconds, 55° C. for 30 seconds, 72° C. for 45 seconds} and the final 72° C. for 7 minutes. Thereafter, a genome fragment derived from hepatitis E virus can be detected by analyzing the product by means of electrophoresis, DNA chip or the like. As a result, the hepatitis E virus can be detected. For example, in the case in which the product is analyzed by electrophoresis, a genome fragment derived from hepatitis E virus can be detected by determining presence/absence of a band derived from HEV genome therein.

In another embodiment of the present invention, the method of detecting HEV virus can be carried out, by using the probe described above, according to a detection method in which the known hybridization method is utilized. In this case, the probe can be labeled with a desired marker substance.

In short, virus detection in the comprehensive manner is possible by using the aforementioned primer and probe for comprehensively detecting HEV. Alternatively, virus detection in the selective manner is possible by using the aforementioned primer and probe for selectively detecting HEV.

For example, by designing a detection system in which a highly conserved region is utilized, the precision of diagnosis of HEV infection can be enhanced. On the other hand, a system for detecting genotype specificity in which system a highly mutated region is utilized can make significant contribution to determination of the infection route and epidemiological study. The system for detecting genotype specificity can also be used for a virus-check test with respect to blood to be used for blood transfusion and a virus-check test of a sample derived from the environment.

According to the present embodiment, virus detection as described above can be achieved not only at the aforementioned nucleic acid level but also at the level of amino acid. For example, the amino acid sequence shown as the SEQ No. 8 and the like can be used as a marker which indicates the presence of HEV, as is the case with the aforementioned polynucleotide. Such an amino acid sequence can be used, for example, for comprehensively diagnosing infection of the virus in an organism including human and for specifically diagnosing the genotype of the virus. Similarly, any of the aforementioned amino acid sequences can be used for the purpose described above.

The gene and polynucleotide derived from HEV-JRA1 according to the present invention described above is a novel substance. The method of detecting HEV by using the polynucleotide or polypeptide and antibody produced from the polynucleotide excels the prior art, in usefulness or advantage thereof in detecting virus genome, virus antibody and the like in a sample. The advantage of the present invention will be described in detail hereinafter by examples.

In order to improve the HEV detection method and the diagnosis technique of HEV infection, it is preferable that the nucleotide sequence information of the gene of such a novel strain as described above is reflected on the detection and diagnosis systems.

8. Chip for Detecting Nucleotide Sequence

According to one embodiment of the present invention, a chip for detecting nucleotide sequence, which chip includes the aforementioned polynucleotide, is provided. Examples of the nucleotide sequence-detection chip of the present embodiment include DNA chip for fluorescent detection, DNA chip of electric current-detection type and the like. However, the nucleotide sequence-detection chip of the present embodiment is not restricted to these examples. The detection method is simplified and made effective, by detecting virus by employing a chip for detecting nucleotide sequence in which chip the aforementioned polynucleotide or a complementary strand thereof is arranged as a probe. The chip for detecting nucleotide sequence can be produced according to the following procedure.

(A) Production of a Chip for Detecting Nucleotide Sequence, to be Used Fluorescent Detection

A polynucleotide according to the present invention or a polynucleotide having a sequence as a portion of the polynucleotide or a polynucleotide having a sequence complementary to any one of the sequences of these polynucleotides, are fixed on a substrate. As the substrate, any substrate of the conventional type e.g., a glass substrate or a silicon substrate can be used. Regarding the fixing means, any suitable means known to one skilled in the art, including a means utilizing a spotter and a means utilizing the general semiconductor technique, can be used.

(b) Production of a Nucleotide Sequence Detection Chip of Electric Current Detection Type

A polynucleotide according to the present invention or a polynucleotide having a sequence as a portion of the polynucleotide or a polynucleotide having a sequence complementary to any one of the sequences of these polynucleotides, are fixed on a substrate, e.g., an electrode substrate, by means of covalent bond, ionic bond, physical adsorption or chemical adsorption. Examples of the DNA chip of electric current detection type include a gene detection device disclosed by JP-B No. 2573443 (issued on Oct. 24, 1996) and the like. However, the chip for detecting nucleotide sequence, of electric current detection type, of the present invention is not restricted to these examples. JP-B No. 2573443 is herein incorporated to the present specification by reference.

According to the present embodiment, detection of virus can be carried out easily and effectively, by detecting virus by using a probe and a chip for detecting gene sequence including the polynucleotides as described above.

9. Protein Chip

In one embodiment of the present invention, a protein chip which allows easy implementation of the detection method described above is provided. In the protein chip, an antibody which recognizes the aforementioned polypeptide is arranged as a probe. By using this protein chip, HEV in a sample can be detected in a simple, easy and efficient manner. Examples of the protein chip include a protein chip for fluorescent detection (which is generally called as “fluorescent colorant-type protein chip”), a protein chip of electric current detection type (which is generally called as “electric potential-type protein chip”) and the like. However, the protein chip of the present embodiment is not restricted to these examples. Examples of the production procedure of the protein chip of the present embodiment will be described below.

(a) Production of a Protein Chip for Fluorescent Detection

A monoclonal antibody of the aforementioned polypeptide is produced in advance. The obtained monoclonal antibody is fixed on a substrate. As the substrate, any substrate of the conventional type e.g., a glass substrate or a silicon substrate can be used. The fixing means may be selected from any suitable means known to one skilled in the art, such as a means utilizing a spotter and a means utilizing the general semiconductor technique. A fluorescent substance, a radioactive isotope, a colorant or the like may be used for labeling for detection.

(b) Production of a Protein Chip of Electric Current Detection Type

A monoclonal antibody of the aforementioned polypeptide is produced in advance. The obtained monoclonal antibody is fixed on a substrate of a type generally used for the conventional electric current detection-type DNA chip, whereby a protein chip of electric current detection type of the present embodiment is produced.

According to the present embodiment, detection of a virus can be carried out easily and efficiently, by detecting virus by using a protein chip including the aforementioned antibody.

Alternatively, the aforementioned polypeptide, instead of the antibody, may be fixed on a substrate, for producing a chip, so that this chip is used for detecting an antibody specific to the polypeptide.

10. Diagnosis System

By using the novel HEV strain, the polynucleotide and the polypeptide as described above according to the present invention, it is possible to diagnose: whether or not the object has been infected with HEV; and/or, if the object has been infected with HEV, to which genotype the HEV belongs; and/or whether or not the object has been infected with fulminating HEV.

The diagnosing method described above includes: for example, collecting a sample from the object by a known method; optionally carrying out purification of the sample and/or amplification of the nucleic acid; and obtaining a nucleic acid sample by purification. Thereafter, the targeted diagnosis can be carried out by using the polypeptide of the present invention, through detecting presence/absence of amplification of the targeted sequence or detecting presence/absence of hybridization. Or, the targeted diagnosis can be done by employing the aforementioned polypeptide of the present invention as a polypeptide for detecting antibody and utilizing the antigen-antibody reaction.

11. Vaccine for Preventive Purpose

In one embodiment of the present invention, a vaccine for HEV is provided. The vaccine can be prepared by using at least one type of polypeptide selected from the immunogenic polypeptides derived from HEV containing nucleic acid coded by (i.e., derived from) the aforementioned nucleotide sequence of the novel HEV virus of the present invention. For example, polypeptide of the ORF2 region of any of the aforementioned novel strains may be used. Production of vaccine containing the immunogenic polypeptide can be carried out by using any of the suitable known methods.

12. Drug Design

The genetic information derived from the aforementioned novel HEV is useful for development and improvement of antiviral agents. Such development and improvement of antiviral agents can be carried out, for example, by following process, by using the known method of drug designing generally called “post-genome drug designing”.
(1) The tertiary structure of enzyme proteins of a plurality of types, coded by ORF1 of the HEV genome, is actually measured by X-ray diffraction and/or NMR analysis of the expressed proteins. Alternatively, the tertiary structure of the enzyme proteins may be obtained by simulation on a computer, only on the basis of the information of the amino acid sequence. The “candidate” domain which can be the target of the drug (which domain will be referred to as “receptor domain” hereinafter), of the enzyme protein, is searched on the information obtained by the measurement or the simulation.
(2) With regards to a drug whose inhibitory action against the enzymic activity of HEV ORF1 protein has been confirmed, a compound which has already been synthesized, although the inhibitory effect thereof against the enzymic activity of HEV ORF1 protein has not been confirmed yet, a compound which has not been synthesized yet but whose structural formula has been known, and an imaginary compound having a novel structure (each of which drug or compound will be referred to as “ligand” hereinafter), the tertiary structures thereof are inputted into a computer, respectively.
(3) By using the data inputted into a computer in the aforementioned process (2), whether or not the receptor domain (“candidate” region) determined in the aforementioned process (1) can be three-dimensionally bound to any of the ligands of the aforementioned process (2) is determined through three-dimensional docking analysis. An effective combination of a ligand with the receptor domain is selected on the basis of the aforementioned determination. Alternatively, improvement of the ligand molecule necessary for causing more effective docking is simulated by the computer.
(4) A ligand obtained by the aforementioned process (3) (i.e., a ligand selected or improved by the process (3)) is likely to function as an effective antiviral agent which suppresses or inhibits proliferation of HEV.

HEV is a virus which includes a number of variants. Therefore, in order to develop and/or improve an antiviral agent being equally effective to all of the variant strains (or as many strains as possible) of HEV, it is necessary to obtain the information of nucleotide sequence of all the existing strains (or as many existing strains as possible) of HEV. According to the aforementioned aspects of the present invention, an advantageous effect is achieved in obtaining such information of nucleotide sequence.

Any of the ORF1 regions derived from the aforementioned novel HEV strains may be utilized in the manner described above. A drug obtained by the aforementioned method of designing drug is also included within the scope of the present invention.

13. Assay Kit

In the present invention, the polynucleotide, polynucleotide fragment, polynucleotide probe, polynucleotide primer, polypeptide, antigen and antibody of any of the aforementioned embodiments may be provided as a kit in an appropriate manner, in accordance with the application purpose. For example, in order to produce a chip for detecting nucleotide sequence, a kit may be formed by combining the polynucleotide/polynucleotide's with a substrate and/or various reagents and/or a marker substance. Further, a kit may be formed by combining the polynucleotide probe with a reaction container and/or salts for buffer solution and/or other necessary reagents. For example, a kit may be formed by combining the polynucleotide primer with a reaction container and/or salts for buffer solution and/or other necessary reagents e.g., polymerase and/or a substrate. Note that the kit provided according to the present invention is not restricted to the aforementioned examples or combinations, but kits of other types may be provided according to necessity by combining the polynucleotide/polypeptide/antigen/antibody components of the present invention with various substances. The application purpose of the kit is not restricted to the aforementioned examples, either, and a kit for any of the application purposes or objects described above may flexibly be formed. Such a kit as described above is also included within the scope of the present invention.

Examples with regards to HEV genome detection according to the present invention will be described hereinafter. It should be noted that the following description is provided only for a demonstrational purpose and by no means restricts the scope of the present invention.

### EXAMPLE 1

### Detection of HEV Genome RNA by RT-PCR Method

1. Primer

In the RT-PCR of the present example, four types of oligonucleotide primers respectively having following nucleotide sequences, selected from the nucleotide sequences derived from HEV-JRA1 disclosed at the SEQ No. 1 or the SEQ No. 2 of the sequence list, were used.
#HE5-1 (5′-TCGATGCCATGGAGGCCCA-3′) (sense primer, which corresponds to nt 19-37 of the sequence list 1) (The underlined portion corresponds to the SEQ No. 2 of the sequence list)
#HE5-2 (5′-GCCYTKGCGAATGCTGTGG-3′) (sense primer, which corresponds to nt 105-123 of the sequence list 1) (Y=C or T; K=G or T) (The underlined portion corresponds to the SEQ No. 3 of the sequence list)
#HE5-3 (5′-TCRAARCAGTARGTGCGGTC-3′) (antisense primer, which corresponds to nt 450-469 of the sequence list 1) (R=A or G) (The underlined portion corresponds to the SEQ No. 4 of the sequence list)
#HE5-4 (5′-CATAGCCTCSGCRACATCAG-3′) (antisense primer, which corresponds to nt 541-560 of the sequence list 1) (S=G or C; R=A or G) (The underlined portion corresponds to the SEQ No. 5 of the sequence list)
   2. Detection Method

First, nucleic acid was extracted from 50 μL of the serum collected from the patient, by using SMITEST EX R & D (Genome Science Laboratories). #HE5-4 as an antisense primer was added to the extracted nucleic acid. The nucleic acid was reacted with the added antisense primer at 37° C. for 30 ° minutes, under the presence of polymerase MMLV-RT (Stratagene), whereby the synthesis of cDNA (i.e., the reaction of reverse transcription from RNA to DNA) was carried out.

Next, the cDNA synthesized as described above was subjected to nested PCR by using the aforementioned four types of primers and Fast Start Taq DNA Polymerase (Roche Co., Ltd.). During the nested PCR, the temperature was changed such that the initial 95° C. lasted for 4 minutes was followed by 30 cycles of {95° C. for 30 seconds, 55C for 30 seconds, 72° C. for 45 seconds} and the final 72° C. for 7 minutes. The product obtained as a result of the PCR reaction was subjected to electrophoresis by using agarose gel. Presence/absence of a band derived from HEV genome having length of 365 bp was checked.

3. Result and Discussion

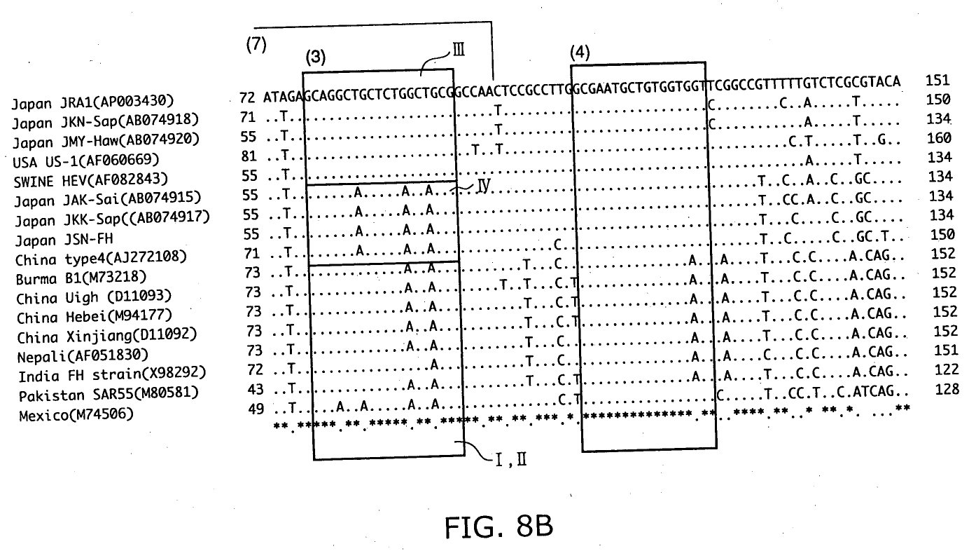

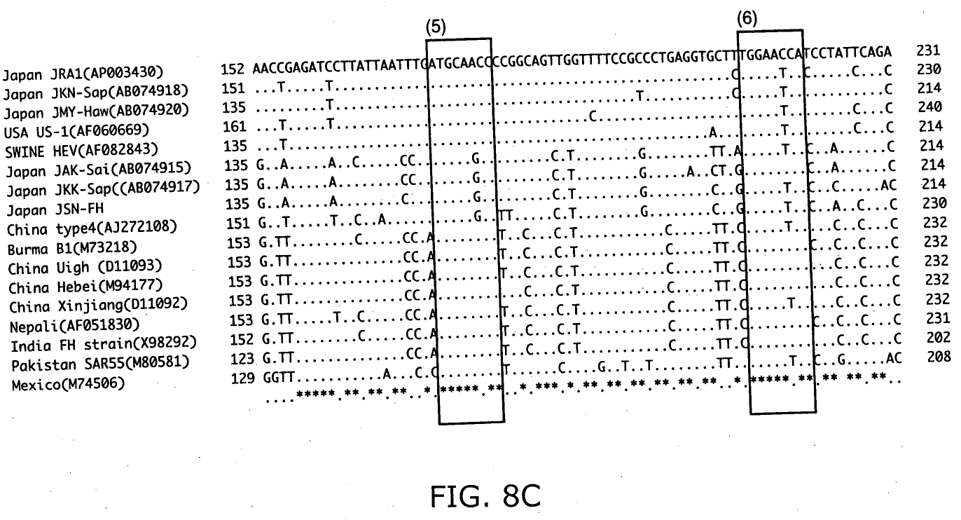

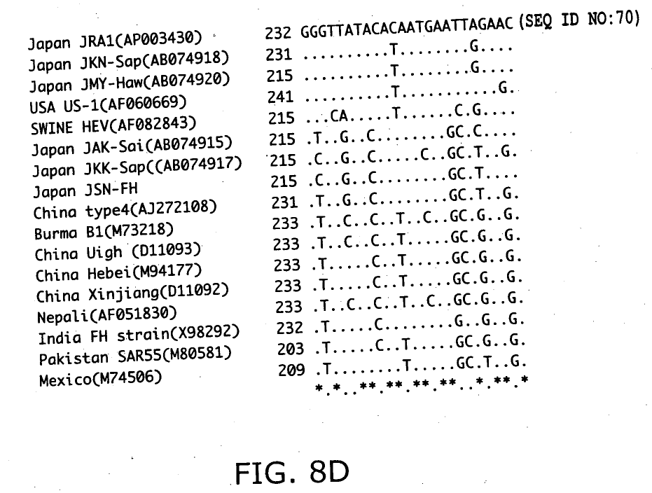

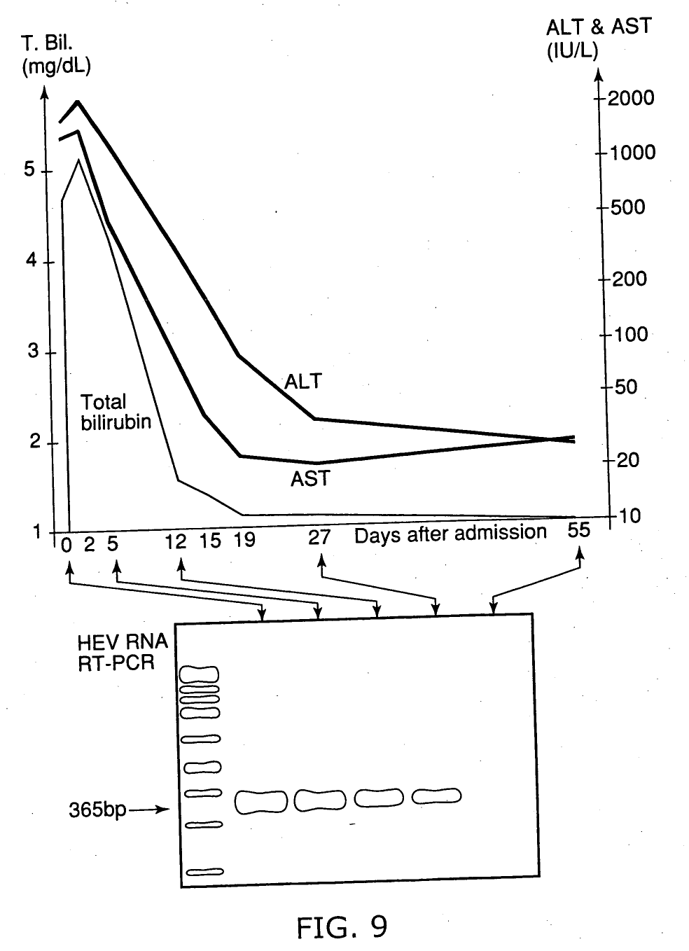

FIG. 9 shows the result of detecting HEV genome RNA by the aforementioned method, in a serum sample successively collected from a Japanese acute hepatitis patient. The graph of FIG. 5A shows changes in the values of liver function test which indicate the progress of hepatitis. The abbreviations of the terms used in FIG. 5A are as follows. “AST” represents aspartic aminotransferase. “ALT” represents alanine aminotransferase. “Total bilirubin” represents the concentration of bilirubin as a whole. The photograph of FIG. 5A shows the result of electrophoresis of the PCR product, derived from the serum collected from the same patient at the timing corresponding to the graph (i.e., by RT-PCR: the reverse transcription polymerase chain reaction method). The X-axis represents the days during which the patient was hospitalized.

In the case shown in FIG. 5A, HEV RNA was continually detected in the serum of the patient, from the initial stage of the disease, throughout the hospitalized period exceeding 27 days. This is an epoch-making discovery which denies the conventional knowledge, because it has conventionally been considered, as common sense, that HEV RNA appears in blood only during a very short period until AST, ALT and Total bilirubin reach the peaks thereof (Purcell, R. H. In Fields Virology, eds. Fields, B. N., Knipe, D. M., & Howley, P. M. Lippincott-Raven, Philadelphia, 1996, 3^{rd} Ed., Vol. 2, pp. 2831-2843). The results shown in FIG. 5A indicate that the detection system of HEV genome RNA, which can be constructed according to the present invention, detects HEV genome RNA with very high sensitivity and that the clinical data obtained from the analysis using such a detection system effectively and thus usefully deepens the academic understanding of HEV infection.

### EXAMPLE 2

### Isolation of the Novel HEV Strain

Tests were conducted for seven acute hepatitis E patients of seven cases. Among the seven cases, the patients of five cases in which JHA-Sap, JKK-Sap, JKN-Sap, JMY-Haw and JSY-Sap were isolated had lived in Hokkaido. The patients of two cases in which JAK-Sai and JMM-Sai were isolated had lived in Saitama prefecture. Each patient developed the disease in an isolated manner i.e., with no contact with other patients regarding both time and place. Further, the case of each patient had nothing to do with any local epidemic of the disease. In six of the seven cases, patients had not been abroad recently. Only the patient from whom JMY-Haw was isolated had been to Hawaii as a tourist one month before developing the disease. The serum sample was collected from the patient at the acute state, frozen at a temperature of −20° C. or below and stored until the virological analysis was carried out.

The HEV sequence was determined, basically according to the method proposed by Takahashi (Takahashi K, Iwata K, Watanabe N, et al. Virology 2001; 287:9-12), with some improvements added thereto. The nucleic acid sample was extracted from 25 mL of serum by using a commercially available kit for nucleic acid extraction (SMITEST EX-R & D (Genome Science Laboratories)). The first strand cDNA was synthesized at 37° C. for 30 minutes by using the reverse transcripase of Moloney mouse leukemia virus (produced by Stratagene Co., Ltd.). As the antisense primer, a mixture of HE5-4 (5′-CATAGCCTCSGCRACATCAG-3′; NT541-560)(SEQ ID No. 61), and HE5-5 (5′-CATYGCCTCSGCAACATCGG-3′; nt541-560, (SEQ ID No. 62), the positions of the respective nucleotides correspond to those of HEV-JRA1 strain) was used. Next, the obtained cDNA was subjected to the nested polymerase chain reaction (referred to as “PCR” hereinafter). For the first round PCR, a mixture of Fast Start Taq DNA Polymerase (produced by Roche Co., Ltd.), an outer-side sense primer HE5-1 (5′-TCGATGCCATGGAGGCCCA-3′; nt 19-37),(SEQ ID No. 58), and an outer-side antisense primer HE5-4/HE5-5 (with regards to the sequences thereof, refer to the aforementioned description), was used. Subsequently, for the second round PCR, a mixture of an inner-side sense primer HE5-2 (5′-GCCYTKGCGAATGCTGTGG-3′; nt 105-123),(SEQ ID No. 59), an inner-side antisense primer HE5-3 (5′-TCRAARCAGTARGTGCGGTC-3′; nt 450-569), (SEQ ID No. 60) and HE5-6 (5′- TYAAAACAGTAGGTTCGATC-3′; nt 450-469), (SEQ ID No. 63), was used. In the aforementioned PCR, the temperature was changed such that the initial 95° C. lasted for 4 minutes was followed by 30 cycles of {95° C. for 30 seconds, 55° C. for 30 seconds, 72° C. for 45 seconds} and the final 72° C. for 7 minutes.

As a result, the 326-nt region was amplified from ORF1 of the HEV genome. Next, the sequencing process was carried out by using “Dye Terminator Cycle Sequencing FS Ready Reaction Kit” (produced by Perkin-Elmer Applied Biosystems Co., Ltd.) and “373A DNA sequencer” (produced by Applied Biosystem Co., Ltd.).

The sequences obtained from the strains isolated (i.e., derived) from the Japanese patients were compared with the sequences of the strains isolated from patients of various foreign nationalities. A computer software (GENETYX-MAC version 10.1 (Software Development Co., Ltd.) was used for comparison.

The result of the comparison described above is shown in FIG. 7. When the 326-nt ORF1 sequences of the seven strains endemic to Japan were compared with each other, the seven strains were classified into three main groups. JMM-Sai sequence exhibited homology of 73.0% to 75.7% with respect to each of the other six isolated strains. JKN-Sap, JHA-Sap and JMY-Haw exhibited relatively high homology of 95.4% to 98.8% with respect to the strains belonging to the same group, but exhibited relatively low homology of 73.0% to 79.1% with respect to the isolated strains belonging to other groups. JSY-Sap, JKK-Sap and JAK-Sai exhibited relatively high homology of 89.0% to 99.7% with respect to each other, but exhibited relatively low homology of 74.8% to 78.8% with respect to the other four strains. The entire-length genome of JRAL strain as the prototype had homology of less than 90% with respect to these isolated strains.

### EXAMPLE 3

### Comprehensive Detection of HEV

First, blood was collected from a plurality of patients. Nucleic acid was extracted from 50 μL of the serum collected from each patient, by using SMITEST EX R & D (Genome Science Laboratories). 5′-gcagaccacrtatgtgktcg-3′(SEQ No. 32) and 5′-ccacrtatgtggtcgaygcc-3′(SEQ No. 33) as the sense primers, as well as 5′-acmarctgscgrggytgcat-3′(SEQ No. 34) and 5′-cgytgratwggrtgrttcca-3′(SEQ No. 35) as the antisense primers were added to each of the extracted nucleic acid. Each nucleic acid was reacted with the added sense primer and antisense primer at 37° C. for 30 minutes, under the presence of polymerase One-step RT.-PCR (Stratagene), whereby the synthesis of cDNA (i.e., the reaction of reverse transcription from RNA to DNA) was carried out.

Next, each of the cDNA synthesized as described above was subjected to nested PCR by using: Fast Start Taq DNA Polymerase (Roche Co., Ltd.); 5′-tgktcgaygccatggaggc-3′(SEQ No. 36), 5′-tgktcgaygccatggaggc-3′(SEQ No. 37) and 5′-aygccatggaggcccaycag-3′(SEQ No. 38) as the sense primer; and 5′-ckracyaccacagcattcgc-3′(SEQ No. 39) and 5′-ggcckracyaccacagcatt-3′(SEQ No. 40) as the antisense primer. During the nested PCR, the temperature was changed such that the initial 95° C. lasted for 4 minutes was followed by 30 cycles of {95° C. for 30 seconds, 55° C. for 30 seconds, 72° C. for 45 seconds} and the final 72° C. for 7 minutes. The PCR product obtained as a result of the PCR reaction was subjected to electrophoresis by using agarose gel.

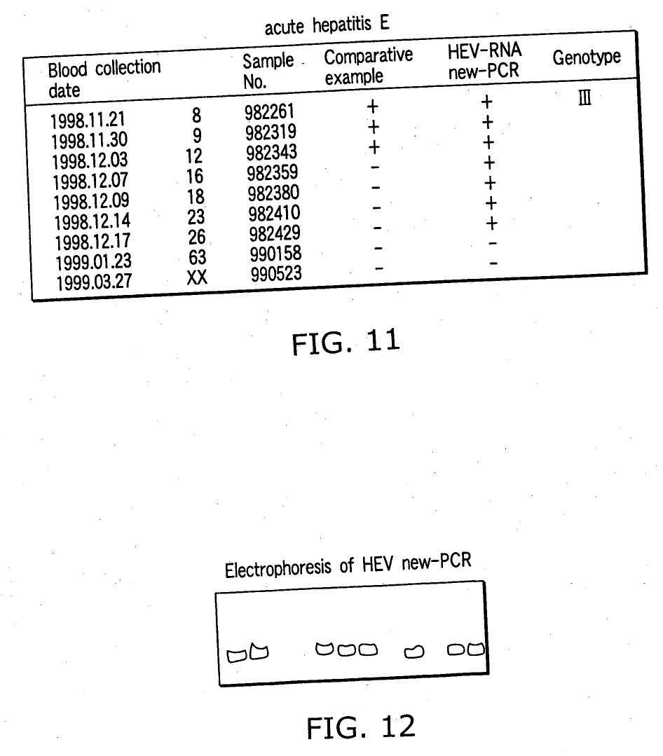

As a result, amplified products were obtained by the aforementioned method of the present example, for 8 cases of the total 37 cases. For the 8 cases in each of which a band was obtained as a result of electrophoresis, the obtained bands are schematically shown in FIG. 12. Each band obtained for each sample was observed at different positions, as shown in FIG. 12. This result indicates that, in the above-described 8 cases, the plural primers used therein acted, as some different combinations of the primers, on the HEV genome in the sample and effected amplification. In other words, the result indicates that the genotypes of HEV detected in the HEV-positive patients are different from each other.

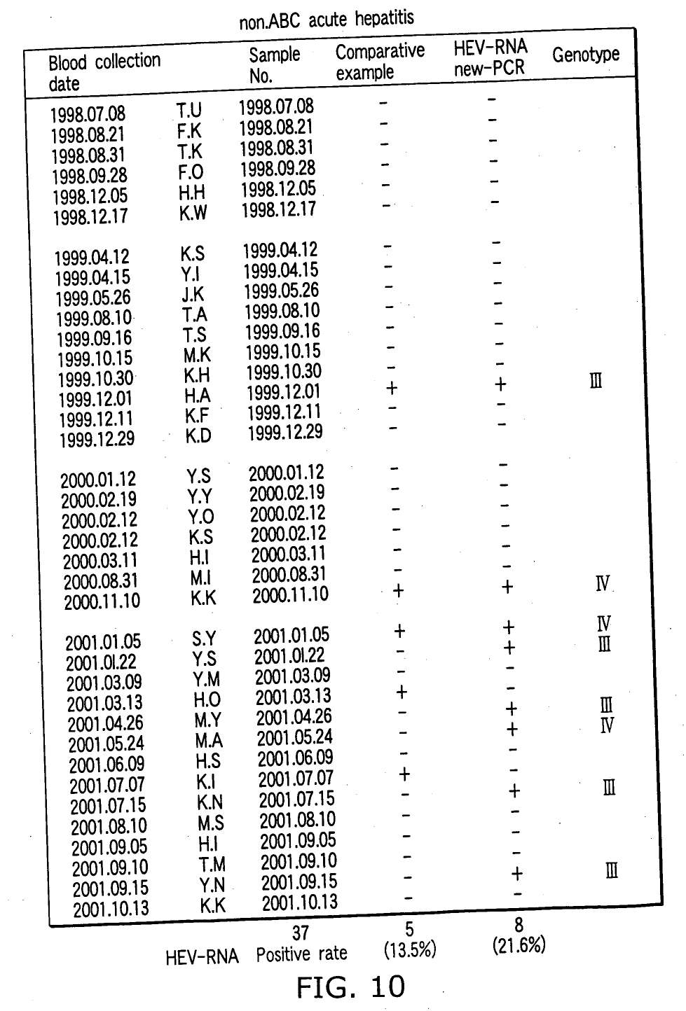

The amplification reaction was carried out for each of the 37 samples of the non-A,B,C type acute hepatitis patients. The result of the amplification is summarized in FIG. 10, in a manner that the result obtained by the conventional method is compared with the result obtained by the method of the present example. The column “Blood collection date” of FIG. 10 represents the date when blood was collected from each patient. The alphabet letters right next to “Blood collection date” represent the initials of the patient. “Sample No.” represents the serial sample number used in the hospital. The column “Comparative example” of FIG. 10 indicates presence/absence of HEV detection when amplification was effected by using the conventional primer. The column “new-PCR ” indicates presence/absence of HEV detection by the method of the present example. In each column of “Comparative example” and “new-PCR”, “+” indicates that HEV was detected and “−” indicates that HEV was not detected. As shown in FIG. 10, the method of the present example was capable of detecting the virus strains which the conventional primer failed to detect, although the same amplification and electrophoresis processes were conducted in the two cases.

### EXAMPLE 4

### Follow-Up Check of a Patient

For a patient who developed acute hepatitis E, presence/absence of the virus in blood of the patient was successively checked. As the measuring method, the method according to the present invention i.e., the method described in example 3 and the conventional method described in example 3 were employed. The result is shown in FIG. 11. The leftmost column of FIG. 11 represents the date when blood was collected. The number right next to “Blood collection date” indicates the period (days) counted from the day when the patient was hospitalized. “Sample No.” of FIG. 11 represents the serial sample number used in the hospital. The column “Comparative example” of FIG. 11 indicates presence/absence of HEV detection when amplification was effected by using the conventional primer. The column “new-PCR” indicates presence/absence of HEV detection by the method of the present example. In each column of “Comparative example” and “new-PCR”, “+” indicates that HEV was detected and“−” indicates that HEV was not detected.

From the result of FIG. 11, it is understood that the method using the primer of the present invention enables detection of virus, even when the virus in blood has been reduced to such a degree that the conventional method can no longer detect the virus.

### EXAMPLE 5

### Genotype Identification of HEV

Next, for each of the products obtained as a result of amplification in example 3, the genotype thereof was identified by using a probe-fixed plate as described below.

(1) Probe-Fixed Plate

A probe of 53 mer (5′-aaggctcctggcrtyactactgcyatwgagcaggcwgctctrgcwgcggccaa-3′),(SEQ ID No. 64), a probe for HEV-I, II (5′-ctcctggcatcactactgc-3′), (SEQ ID No. 65), a probe for HEV-III (5′-ctcctggcattactactgc-3′)(SEQ ID No. 66), and a prove for HEV-IV (5′-ctcctggcgtcactactg-3′)(SEQ ID No. 67), were solid-phase fixed, respectively, on a well of a commercial microtiter platewell.

(2) Genotype Identification by Hybridization Assay

Each of the products obtained as a result of amplification in example 3 was added to the probe-fixed plate prepared in the aforementioned (1), whereby hybridization was effected. Thereafter, optical density (O. D.) of each well was measured.

The results are shown in FIGS. 13A-13E. The graphs of FIG. 13A, FIG. 13B, FIG. 13C, FIG. 13D and FIG. 13E show the result of genotype identification conducted for the HEV having different genotypes, i.e., JRA1 strain, JKN-Sap strain, JKK-Sap strain, JAK-Sai strain and JMM-Sai strain, respectively. In all of the graphs, the X-axis represents the HEV-I, II solid phase well, the HEV-III solid phase well, and the HEV-IV (54 mer primer) solid phase well, from left to right in this order. In the case of genotype III, only the O. D. of the well in which the probe for HEV-III had been solid-phase fixed was distinctly high. In the case of genotype IV, only the O. D. of the well in which the probe for HEV-IV had been solid-phase fixed was distinctly high. In the case of genotype I, the well in which the probe for HEV-I, II had been solid-phase fixed exhibited relatively high O. D., as compared with the other wells. On the basis of the indexes described above, the genotype of the virus in the serum of each patient was identified by using the corresponding probe. The result of the genotype identification was shown in the column “genotype” of FIG. 10. As is obvious from the result shown in FIG. 10, it has been demonstrated that genotype identification of HEV collected from a patient can be easily carried out by using the probe-according to the present invention.

The total contents of all the references cited in the present specification are incorporated in the present specification, by reference.

We claim:

1. An isolated and purified polynucleotide probe, which can be hybridized with nucleotides from at least one hepatitis E virus selected from the group consisting of
JSN-FH,
JKN-Sap,
JMY-Haw,
JAK-Sai,
JKK-Sap
and JRA1,
wherein the sequence of the polynucleotide probe comprises at least eight nucleotides selected from the group consisting of sequences represented by the
19th-37th nucleotides of SEQ ID No. 15,
the 111th-127th nucleotides of SEQ ID No. 15,
the 174th-181st nucleotides of SEQ ID No. 15,
the 213th-220th nucleotides of SEQ ID No. 15,
the 48th-100th nucleotides of SEQ ID No. 15, and
complementary strands thereof.

2. A probe assay kit comprising the polynucleotide probe of claim 1.

3. A chip for detecting a polynucleotide sequence, wherein the polynucleotide probe of claim 1 has been solid-phase fixed to the chip.

4. An isolated and purified primer pair which can amplify nucleotides from at least one hepatitis E virus selected from the group consisting of
JSN-FH,
JKN-Sap,
JMY-Haw,
JAK-Sai,
JKK-Sap,
and JRA1,
wherein the sequences of the purified primer pair comprise at least eight nucleotides selected from the group consisting of sequences represented by
19th-37th nucleotides of SEQ ID No. 15,
the 111th-127th nucleotides of SEQ ID No. 15,
the 174th-181st nucleotides of SEQ ID No. 15,
the 213th-220th nucleotides of SEQ ID No. 15,
the 48th-100 th nucleotides of SEQ ID No. 15, and
complementary strands thereof.

5. A PCR assay kit, comprising the primer pair of claim 4.

6. Isolated and purified plural pairs of primer which can amplify nucleotides from at least one hepatitis E virus selected from the group consisting of
JSN-FH,
JKN-Sap,
JMY-Haw,
JAK-Sai,
JKK-SAP,
and JRA1,
wherein the sequences of the purified plural pairs of primer comprise at least eight nucleotides selected from the group consisting of sequences represented by
19th-37th nucleotides of SEQ ID No. 15,
the 111th-127th nucleotides of SEQ ID No. 15,
the 174th-181st nucleotides of SEQ ID No. 15,
the 213th-220th nucleotides of SEQ ID No. 15,
the 48th-100th nucleotides of SEQ ID No. 15, and
complementary strands thereof.

7. A PCR assay kit, comprising the plural pairs of primer of claim 6.
